(19) United States
(12) Patent Application Publication
(10) Pub. No.: US 2020/0010847 A1
(43) Pub. Date: Jan. 9, 2020

(54) COMPOSITIONS AND SYSTEMS FOR CONFERRING DISEASE RESISTANCE IN PLANTS AND METHODS OF USE THEREOF

(71) Applicant: INDIANA UNIVERSITY RESEARCH AND TECHNOLOGY CORPORATION, Indianapolis, IN (US)

(72) Inventors: Roger William INNES, Bloomington, IN (US); Matthew David HELM, Indianapolis, IN (US)

(21) Appl. No.: 16/577,546

(22) Filed: Sep. 20, 2019

Related U.S. Application Data
(63) Continuation-in-part of application No. 16/395,070, filed on Apr. 25, 2019.
Application No. 16/395,070 is a continuation-in-part of application No. PCT/US2018/025511, filed on Mar. 30, 2018.
(60) Provisional application No. 62/482,074, filed on Apr. 5, 2017.

(51) Int. Cl. C12N 15/82; C07K 14/415

(52) U.S. Cl. CPC C12N 15/8281; C07K 14/415; C07K 2319/50; C12N 15/8282; C12N 15/8283

(57) ABSTRACT

Compositions, systems and methods are provided for conferring disease resistance to plant pathogens that use proteases to target plant substrate proteins inside plant cells. Briefly, the compositions, systems and methods are based upon plant substrate proteins that are targeted by pathogen-specific proteases and that activate nucleotide binding site-leucine rich repeat (NB-LRR) disease resistance proteins when cleaved by the protease. These substrate proteins are modified such that the endogenous protease recognition sequence is replaced by a protease recognition sequence specific to a different pathogen protease (i.e., a heterologous protease recognition sequence). The modified plant substrate protein therefore can be used in connection with its corresponding NB-LRR protein to activate resistance in response to cleavage by the heterologous pathogen-specific protease. When activated by the plant pathogen-specific protease, the pair initiates host defense responses thereto, including programmed cell death.

### CROSS-REFERENCE TO RELATED APPLICATION

This application is a Continuation-in-Part of U.S. patent application Ser. No. 16/395,070, filed Apr. 25, 2019, which is a Continuation-in-Part of International Patent Application No. PCT/US2018/025511, filed Mar. 30, 2018, which claims the benefit of priority to U.S. Provisional Patent Application No. 62/482,074, filed on Apr. 5, 2017. The disclosures set forth in the referenced applications are incorporated herein by reference in their entirety.

### STATEMENT REGARDING FEDERALLY SPONSORED RESEARCH OR DEVELOPMENT

This invention was made with government support under IOS1551452 awarded by the National Science Foundation. The Government has certain rights in the invention.

### INCORPORATION OF SEQUENCE LISTING

A computer readable form of the Sequence Listing containing the file named “291857_SEQ_ST25.txt” has been submitted electronically in ASCII format and is hereby incorporated by reference in its entirety. Said ASCII copy, created on Apr. 24, 2019, is 179,676 bytes in size.

### BACKGROUND

The present disclosure relates generally to plant genetics and plant molecular biology, and more particularly relates to compositions, systems and methods of conferring disease resistance to soybean plant pathogens that express pathogen-specific proteases based on recognition of the pathogen-specific proteases in the soybean plant cell.

Plant diseases are a serious limitation on agricultural productivity and influence the development and history of agricultural practices. A variety of plant pathogens are responsible for plant diseases including bacteria, fungi, insects, nematodes and viruses.

Incidence of plant diseases can be controlled by agronomic practices that include conventional breeding techniques, crop rotation and use of synthetic agrochemicals. Conventional breeding methods, however, are time-consuming and require continuous effort to maintain disease resistance as plant pathogens evolve. See, Grover & Gowthaman (2003) Curr. Sci. 84:330-340. Likewise, agrochemicals increase costs to farmers and cause harmful effects on the ecosystem. Because of such concerns, regulators have banned or limited the use of some of the most harmful agrochemicals.

Agricultural scientists now can enhance plant pathogen resistance by genetically engineering plants to express anti-pathogen polypeptides. For example, potatoes and tobacco plants have been developed that exhibit an increased resistance to foliar and soil-borne fungal pathogens. See, Lorito et al. (1998) Proc. Natl. Acad. Sci. USA 95:7860-7865. In addition, transgenic barley has been developed that exhibit an increased resistance to fungal pathogens. See, Horvath et al. (2003) Proc. Natl. Acad. Sci. USA 100:364-369. Moreover, transgenic corn and cotton plants have been developed to produce Cry endotoxins. See, e.g., Aronson (2002) Cell Mol. Life Sci. 59:417-425; and Schnepf et al. (1998) Microbiol. Mol. Biol. Rev. 62:775-806. Other crops, including potatoes, have been genetically engineered to contain similar endotoxins. See, Hussein et al. (2006) J. Chem. Ecol. 32:1-8; Kalushkov & Nedved (2005) J. Appl. Entomol. 129:401-406 and Dangl et al. (2013) Science 341: 746-751.

In light of the significant impact of plant pathogens on the yield and quality of plants, additional compositions, systems and methods are needed for protecting plants, and in particular, soybean plants, from plant pathogens.

### SUMMARY

Engineering of novel disease resistance traits in monocot crops such as barley, wheat, rice, sorghum, and corn is accomplished by modifying endogenous PBS1 genes in these crops so that the proteins they encode are cleaved by proteases from pathogens of interest. For example, engineering recognition of proteases from the fungal pathogen wheat stripe rust was accomplished by modifying wheat PBS1 genes. Engineering resistance to many other fungal and viral pathogens, including Fusarium, a major problem for corn growers, is disclosed.

In previous reports by the inventors, the sequence of the PBS1 gene from Arabidopsis thaliana was modified to enable cleavage by specific pathogen-derived proteases such as the Nla proteases from Turnip mosaic virus and Soybean mosaic virus. Cleavage of the PBS1 protein activates disease resistance in Arabidopsis. Modification of a soybean PBS1 gene was found to enable cleavage by SMV Nla protease and activation of disease resistance in soybeans.

Multiple barley varieties indeed recognize and respond to AvrPphB protease activity, and barley also contains PBLs that are cleaved by AvrPphB. Using newly developed NAM resources, the AvrPphB response was mapped to a single segregating locus on chromosome 3HS and an NLR gene was identified that designated AvrPphB Response 1 (Pbr1). PBR1 mediates AvrPphB recognition. Using transient expression assays in Nicotiana benthamiana, it was confirmed that PBR1 associates with PBS1 homologs in planta. Phylogenetic analyses indicate that Pbr1 and RPS5 are not orthologous, hence the ability to recognize AvrPphB protease activity has evolved independently in monocots and dicots. Wheat varieties also recognize AvrPphB protease activity and harbor an ortholog of Pbr1 in a syntenic position on chromosome 3B, suggesting that the PBS1-decoy system might be deployed in barley and in wheat.

The present disclosure demonstrates that a PBS1 protein from barley (a monocot crop) was modified so that it is cleaved by the Nla protease of Wheat streak mosaic virus. Disease resistance was activated in many barley varieties in response to proteolytic cleavage of a barley PBS1 protein. This resistance response is mediated by a barley disease resistance protein identified and named AvrPphB Recognition 1 (PBR1). Thus, engineered recognition of diverse pathogen proteases in barley, and other monocot crops such as rice, wheat and corn is effected by modifying the native PBS1 genes in these crops. To support this assertion, the PBR1 gene is conserved in wheat, and cleavage of wheat PBS1 proteins activates resistance in wheat. This disclosure thus presents modification of PBS1 genes in all monocot plant species as enabling cleavage by pathogen proteases and activation of disease resistance.

Compositions, systems and methods are provided for conferring disease resistance to plant pathogens that express pathogen-specific proteases by modifying at least one member of a protein pair used by plants to detect the pathogen-specific proteases. These protein pairs enable plants to activate endogenous defense systems in response to the pathogen-specific proteases. Briefly, the compositions, systems and methods are based upon a protein pair in which one member of the pair is a nucleotide binding-leucine rich repeat (NB-LRR) disease resistance protein and the other member of the pair is a substrate protein of a pathogen-specific protease that physically associates with its native/corresponding NB-LRR protein and that activates the NB-LRR protein when cleaved by the pathogen-specific protease. The specificity of such pairs for a given pathogen-specific protease can be engineered by replacing an endogenous protease recognition sequence in the substrate protein with a recognition sequence for a pathogen-specific protease of interest (i.e., a heterologous protease recognition sequence).

The compositions include recombinant nucleic acid molecules having a nucleotide sequence that encodes a modified substrate protein (also referred to herein as a “fusion protein”) of a pathogen-specific protease, where the modified substrate protein has a heterologous protease recognition sequence. The heterologous protease recognition sequence can be within, for example, an exposed loop of the modified substrate protein. Optionally, the recombinant nucleic acid molecule can have a nucleotide sequence that encodes a NB-LRR protein so that the nucleic acid molecule encodes the protein pair. For example, in one embodiment, a recombinant nucleic acid molecule having a nucleotide sequence that encodes the NB-LRR protein can be co-transformed with the recombinant nucleic acid molecules having a nucleotide sequence that encodes a modified substrate protein of a pathogen-specific protease so that the modified substrate protein and the NB-LRR protein are co-expressed. The NB-LRR protein can associate with, and can be activated by, the modified substrate protein of the pathogen-specific protease.

The compositions also include isolated, modified substrate proteins of pathogen-specific proteases as described herein, as well as active fragments and variants thereof.

The compositions also include nucleic acid constructs, such as expression cassettes and vectors, having a nucleotide sequence that encodes a modified substrate protein of a pathogen-specific protease as described herein operably linked to a promoter that drives expression in a plant cell, plant part or plant. Such a nucleic acid construct can be used to provide a modified substrate protein to a plant cell, plant part or plant that natively expresses the corresponding NB-LRR protein. The modified substrate protein can associate with, and can activate, the NB-LRR protein.

Optionally, the constructs, including expression cassettes and vectors, can include a nucleotide sequence that encodes a NB-LRR protein operably linked to a promoter that drives expression in a plant cell, plant part or plant. The nucleic acid constructs having a nucleotide sequence that encodes a modified substrate protein of a pathogen-specific protease and the nucleic acid constructs having a nucleotide sequence that encodes a NB-LRR protein can be co-expressed in a plant cell, plant part or plant. The NB-LRR protein can associate with, and can be activated by, the modified substrate protein of the pathogen-specific protease. Such a nucleic acid construct can be used to provide the protein pair to a plant cell, plant part or plant that does not natively express both members of the protein pair.

The compositions also include transformed plant cells, plant parts and plants having a nucleotide sequence that encodes at least one modified substrate protein of a pathogen-specific protease as described herein operably linked to a promoter that drives expression in a plant cell, plant part or plant. Optionally, the plant cells, plant parts and plants are transformed to include a nucleotide sequence that encodes a NB-LRR protein operably linked to a promoter that drives expression in the plant cell, plant part or plant. The NB-LRR protein can associate with, and can be activated by, the modified substrate protein of the pathogen-specific protease.

The systems include a nucleic acid construct having a nucleotide sequence for a first promoter that drives expression in a plant cell, plant part or plant operably linked to a nucleotide sequence that encodes a modified substrate protein of a pathogen-specific protease as described herein and a nucleotide sequence for a second promoter that drives expression in a plant cell, plant part or plant operably linked to a nucleotide sequence that encodes a NB-LRR protein. The NB-LRR protein can associate with, and can be activated by, the modified substrate protein. Such systems can be used to provide the protein pair to a plant cell, plant part or plant that does not natively express both members of the protein pair.

The systems also include a first nucleic acid construct having nucleotide sequence for a promoter that drives expression in a plant cell, plant part or plant operably linked to a nucleotide sequence that encodes a modified substrate protein of a pathogen-specific protease as described herein, and a second nucleic acid construct having a nucleotide sequence for a promoter that drives expression in a plant cell, plant part or plant operably linked to a nucleotide sequence that encodes a NB-LRR protein. Additional nucleic acid constructs also can be included in the system, where each construct has a nucleotide sequence that encodes a distinct modified substrate protein, each having a heterologous recognition sequence for a separate pathogen-specific protease. Although each modified substrate protein has a heterologous recognition sequence distinct from one another, each can associate with, and can activate, the NB-LRR protein. Alternatively, the first nucleic acid construct can encode more than one modified substrate protein, where each modified substrate protein has a heterologous recognition sequence distinct from one another and where each can associate with, and can activate, the NB-LRR protein. Alternatively, the second nucleic acid construct can encode one or more modified substrate proteins, where each modified substrate protein has a heterologous recognition sequence distinct from one another and where each can associate with, and can activate, the NB-LRR protein. Such systems can be used to provide the protein pair to a plant cell, plant part or plant that does not natively express the protein pair or can be used to provide more than one modified substrate protein to a plant cell, plant part or plant.

By way of example, the substrate protein of the pathogen-specific protease can be a PBS1 homolog from Glycine max (soybean) (e.g., PBS1 homolog GmPBS1a (SEQ ID NO:4). GmPBS1b (SEQ ID NO:6), and GmPBS1c (SEQ ID NO:8)). The PBS1 homolog is modified to include a heterologous protease recognition sequence. As understood by those skilled in the art, “PBS1” refers to avrPphB susceptible 1. As understood by those skilled in the art, “avrPphB” refers to the bacterial avirulence from Pseudomonas syringae that encodes the “AvrPphB” polypeptide having a role in plant-P. syringae interactions.

In some embodiments, the present disclosure is directed to the fusion protein encoded by the nucleotide sequence. By way of example, the fusion protein can include a G. max AvrPphB susceptible 1 (GmPBS1) substrate protein and a heterologous pathogen-specific protease recognition sequence. Exemplary GmPBS1 substrate proteins including a heterologous pathogen-specific protease recognition sequence includes proteins having an amino acid sequence of SEQ ID NOs:10, 12 or 14.

In view of the foregoing, the methods include introducing into a plant cell, plant part or plant at least one nucleic acid molecule, construct, expression cassette or vector as described herein to confer disease resistance to plant pathogens that express pathogen-specific proteases.

The compositions, systems and methods therefore find use in conferring disease resistance to plant pathogens by transferring to plant cells, plant parts or plants nucleotide sequences that encode at least one modified substrate protein of a pathogen-specific protease and optionally that encode a NB-LRR protein when such NB-LRR protein is not native to the plant cell, plant part or plant. The pair is thus engineered to be specific for a plant pathogen-specific protease by including in the modified substrate protein a heterologous protease recognition sequence for that plant pathogen-specific protease. When activated by the plant pathogen-specific protease, the pair initiates host defense responses thereto, including programmed cell death.

These and other features, objects and advantages of the present disclosure will become better understood from the description that follows. In the description, reference is made to the accompanying drawings, which form a part hereof.

### BRIEF DESCRIPTION OF THE DRAWINGS

The patent or application file contains at least one drawing executed in color. Copies of this patent or patent application publication with color drawings will be provided by the office upon request and payment of the necessary fee.

The features, objects and advantages other than those set forth above will become more readily apparent when consideration is given to the detailed description below. Such detailed description makes reference to the following drawings, wherein:

FIGS. 1A & 1B. FIG. 1A depicts relations of three genes within the Glycine max genome that encode proteins with (FIG. 1B) significant amino acid homology to Arabidopsis PBS1 (AtPBS1; At5g13160) (SEQ ID NO:16) (also SEQ ID NOS: 4, 6, 8 respectively).

FIG. 2. Depicts modified soybean PBS1 substrate proteins to function as ‘decoys’ for the SMV NIa protease. FIG. 2 discloses SEQ ID NOS 2 and 1, respectively, in order of appearance.

FIGS. 3A & 3B. Depict recognition of AvrPphB in soybean. (FIG. 3A) is a leaf; (FIG. 3B) are test results.

FIGS. 4A & 4B. Depict the effects of activating an AvrPphB-specific R protein in soybean (FIG. 4A) photos of leaves; (FIG. 4B) resistance to Soybean Mosaic Virus (SMV).

FIGS. 5A-5C. Depict the effects of activating RPS5 in Arabidopsis on resistance to Turnip Mosaic Virus (TuMV); (FIG. 5A) graphically illustrates cleavage of PBS1; (FIG. 5B) and (FIG. 5C) show effect on PBS1 leaves.

FIGS. 6A & 6B. Depict that overexpression of PBS1^{TuMV} confers resistance to infection by TuMV. (FIG. 6A) are ultraviolet light images of Arabidopsis plants expressing the PBS1^{TuMV} decoy protein. (FIG. 6B) shows immunoblot analysis of PBS1^{TuMV} and viral protein levels in transgenic lines.

FIGS. 7A & 7B. (FIG. 7A) depicts that AvrPphB was recognized in soybean, barley and wheat; (FIG. 7B) are immunoblots showing cleavage of PBS1 proteins from Arabidopsis (At), soybean (Gm) and barley (Hv) by AvrPphB.

FIG. 8. AvrPphB protease activity elicits a range of responses in barley lines. Representative barley leaves from 12 lines after infiltration with strains of Pseudomonas syringae pv. tomato DC3000(D36E) expressing AvrPphB or a catalytically inactive mutant, AvrPphB(C98S). Primary leaves of ten day old plants were infiltrated using needleless syringe with a bacterial suspension at an OD₆₀₀=0.5 and photographed at 5 dpi. Phenotypes were scored as: N—no response; LC—low chlorosis; C—chlorosis; HC—high chlorosis; HR—hypersensitive reaction. At least six plants were infiltrated with both strains per line over two repeats. Asterisks (*) indicate parental lines of the mapping population families used for GWAS. Responses of all lines tested are recorded in Table 1.

FIG. 9A-9C. Barley contains two PBS1 homologs that are cleaved by AvrPphB. (FIG. 9A) HORVU2Hr1G070690.2 (HvPBS1-1) and HORVU3Hr1G035810.1 (HvPBS1-2) are co-orthologous to Arabidopsis PBS1. Shown is a Bayesian phylogenetic tree generated from the amino acid sequences of Arabidopsis PBS1 (AtPBS1) and closely related barley homologs of AtPBS1. This tree is a subset of (FIG. 16) displaying the proteins most similar to AtPBS1. Branch annotations represent Bayesian posterior probabilities as a percentage. (FIG. 9B) Alignment of the activation segment sequences of AtPBS1 and the barley PBS1 homologs (SEQ ID NOS 17-19, respectively, in order of appearance). The AvrPphB cleavage site is indicated by the arrow. Numbers indicate amino acid positions. (FIG. 9C) Cleavage of HvPBS1-1 and HvPBS1-2 by AvrPphB. HA-tagged barley PBS1 homologs or AtPBS1 were transiently co-expressed with or without myc-tagged AvrPphB, or a protease inactive derivative [AvrPphB(C98S)] in N. benthamiana. Six hours post-transgene induction, total protein was extracted and immunoblotted with the indicated antibodies. Two independent experiments were performed with similar results.

FIG. 10A-10C. Genome wide association study identifies a single locus in the barley genome significantly associated with AvrPphB response. Manhattan plots of the association between SNPs and AvrPphB response of NAM barley lines for (FIG. 10A) all 175 lines and (FIG. 10B) the lines from each HR subpopulation individually. The X-axis shows SNPs in the region graphed, either the whole genome or the interval containing the significant locus in the short arm of Chromosome 3H (3HS). The Y-axis shows the negative logarithm of the p-value for the association. The locations of genes encoding NLRs predicted by NLR-Parser are indicated by open triangles; the blue triangle points to Pbr1, the orange to Goi2. The dotted horizontal line indicates a false discovery rate of 0.05 with Bonferroni correction. (FIG. 10C) Graphical representation of 18 recombinant lines from four additional families used to fine map the AvrPphB response determinant Green indicates regions containing SNPs matching the Rasmusson genotype. Blue indicates regions matching the other parental genotype. Uncolored regions represent the intervals in which it can be concluded the recombination took place, based on the nearest flanking SNPs. Lines labeled in green font display the Rasmusson HR phenotype (1), in blue the other parent phenotype (0).

FIG. 11. RPS5 and PBR1 are phylogenetically distant. Neighbor-Joining phylogenetic tree of the amino acid sequence of the NB-ARC domains from 304 NLRs predicted to be encoded in the barley genome (HORVU), and 15 known Coiled Coil NLRs from Arabidopsis thaliana (At). For simplicity, clades containing >5 predicted NLRs from barley were collapsed, with the number of sequences represented in the adjacent parentheses. Node labels indicate confidence probabilities (shown for those above 50%) from an interior-branch test with 1000 replicates. Triangles indicate predicted protein products encoded within the GWAS interval, and RPS5 is marked with a circle.

FIG. 12A-12D. Sequence and expression polymorphism in Pbr1 across barley lines correlate to AvrPphB response. (FIG. 12A) Schematic illustration of Pbr1.b, the allele in the AvrPphB-responding line Rasmusson, showing the approximate location of a C nucleotide deletion that disrupts the open reading frame in Pbr1.a, the allele in the non-responding line Morex. Below, the Pbr1.b protein product is represented, with the amino acid positions of the CC domain, NB-ARC domain, and LRR domain indicated. (FIG. 12B) PCR amplification from cDNA and genomic DNA (gDNA) of 12 representative lines that differ in their response to AvrPphB, showing expression and primer compatibility, respectively, for Pbr1 and Goi2. cDNA was generated from RNA extracted from 10-day old plants, the same age used for phenotyping in FIG. 8. See FIG. 11 for data from additional lines. (FIG. 12C) A neighbor joining tree showing the sequence relationships of Pbr1 alleles from the barely lines represented in (FIG. 12B) and the (at right) the responses of those lines to AvrPphB. The tree is based on aligned genomic DNA sequence from start codon to stop codon. Nodes are labeled with bootstrap values and the scale bar represents number of base substitutions per site. N, no response; LC, low chlorosis; C, chlorosis; HC, high chlorosis; HR, hypersensitive reaction. FIG. 12D Schematic illustration of the PBR1.b protein annotated with the approximate location of amino acid substitutions present in the predicted protein products of other Pbr1 alleles and the responses of the corresponding barley lines. *C includes all 3 chlorotic responses.

FIG. 13A-13E. Transient co-expression of PBR1.c with AvrPphB induces cell death in N. benthamiana. (FIG. 13A) Schematic representation of the PBR1.b protein product from Rasmusson, an HR line, showing the approximate locations of amino acid substitutions between the PBR1.b protein product and the PBR1.c protein product from CI 16151, a low-chlorosis line. The approximate location of amino acid substitutions present in the predicted protein products of other Pbr1 alleles and the responses of the corresponding barley lines was investigated. (FIG. 13B) Induction of cell death by PBR1.b:sYFP, but not PBR1.c:sYFP, independent of AvrPphB expression when transiently expressed in N. benthamiana. PBR1.b:sYFP or PBR1.c:sYFP were agroinfiltrated into 3-week old N. benthamiana. All transgenes were under the control of a dexamethasone-inducible promoter. A representative leaf was photographed 24 hours post-transgene induction under white light and UV light. Three independent experiments were performed with similar results. (FIG. 13C) Activation of HR by transient co-expression of PBR1.c:sYFP and AvrPphB:myc in N. benthamiana. Agroinfiltrations were used to transiently express combinations of PBR1.c:sYFP, empty vector (e.v.), AvrPphB:myc, and a protease inactive derivative, AvrPphB(C98S):myc. HA-tagged Arabidopsis PBS1 co-expressed with RPS5:sYFP and AvrPphB:myc was used as a positive control. All transgenes were under the control of a dexamethasone-inducible promoter. A representative leaf was photographed 24 hours post-transgene induction under white light and UV light. Three independent experiments were performed with similar results. (FIG. 13D) Electrolyte leakage as a measure of cell death resulting from co-expression of PBR1.c:sYFP with AvrPphB:myc relative to PBR1.c:sYFP with e.v. or AvrPphB(C98S):myc in N. benthamiana leaf discs. The assay was performed using N. benthamiana leaf discs transiently expressing the indicated combinations of constructs. Conductivity is shown as mean±S.D. (n=4). Three independent experiments were performed with similar results. (FIG. 13E) Cleavage of N. benthamiana PBS1 (NbPBS1) by AvrPphB. HA-tagged N. benthamiana PBS1 or AtPBS1 was transiently co-expressed with or without myc-tagged AvrPphB or AvrPphB(C98S) in N. benthamiana. Total protein was extracted six hours post-transgene induction and immunoblotted with the indicated antibodies. Two independent experiments were performed with similar results.

FIG. 14. PBS1 proteins immunoprecipitate with PBR1.c when transiently co-expressed in N. benthamiana. The indicated construct combinations were transiently co-expressed in leaves of 3-week old N. benthamiana plants using agroinfiltration. All transgenes were under the control of a dexamethasone-inducible promoter. Total protein was extracted six hours post-transgene induction. HA-tagged Arabidopsis PBS1 co-expressed with RPS5:sYFP was used as a positive control. The sYFP:LTI6b fusion protein, which is targeted to the plasma membrane (Cutler et al., 2000), was co-expressed with the HA-tagged PBS1 proteins as a negative control. Results are representative of two independent experiments.

FIG. 15A-15C. Recognition of AvrPphB protease activity is conserved in wheat. Responses of wheat cultivars Fielder and Centana infiltrated with (top to bottom) 10 mM MgCl₂ (mock), P. syringae DC3000(D36E) expressing empty vector (e.v.), AvrPphB(C98S), or AvrPphB three days post-infiltration (dpi), photographed under white and UV light. Bacteria (OD₆₀₀=0.5) were infiltrated into the adaxial surface of the second leaf of two-week old seedlings. Three independent experiments were performed with similar results. Responses of all lines tested are recorded in Table 3. (FIG. 15B) Hydrogen peroxide accumulation. Cultivars and treatments assayed were as in panel A. Three dpi, leaf segments were excised from the infiltrated regions, stained with DAB solution, cleared with 70% ethanol, and photographed under white light. This experiment was repeated twice with similar results. (FIG. 15C) Full-length amino acid sequence alignment between barley PBR1.c and the most closely related homolog in wheat, TRIAE_CS42_3B_TGACv1_226949_AA0820360 (TaPBR1) (SEQ ID NOS 20-21, respectively, in order of appearance). Conserved residues and conservative substitutions are highlighted with black and grey backgrounds, respectively. The predicted coiled-coil (CC), nucleotide binding (NB-ARC), and leucine-rich repeat (LRR) domains of TaPBR1 are indicated by pink, green, and cyan bars, respectively. The predicted palmitoylation site is indicated by a purple box.

FIG. 16. Bayesian phylogenetic tree based on amino acid alignment of full-length products of Arabidopsis PBS1 (AtPBS1), all characterized Arabidopsis PBS1-like (AtPBL) genes, and barley PBS1-like (HvPBL) genes homologous to Arabidopsis PBS1. AtPBS1 and AtPBL sequences were obtained from The Arabidopsis Information Resource (TAIR10) website (arabidopsis.org). Homology searches were performed using BLASTp to identify barley amino acid sequences homologous to Arabidopsis PBS1 and PBS1-like proteins. Thirty-two barley protein sequences were identified as homologous to the 29 Arabidopsis sequences used in the analysis. Bayesian phylogenetic trees were generated for the collected sequences using the program MrBayes under a mixed amino acid model. Scale bars indicate amino acid substitutions per site and nodes are labeled with Bayesian posterior probabilities as a percentage. The gray box highlights the clade presented in FIG. 9.

FIG. 17. Full-length amino acid sequence alignment between Arabidopsis PBS1 and the barley PBS1 homologs (SEQ ID NOS 22-24, respectively, in order of appearance). Conserved residues and conservative substitutions are highlighted with black and grey backgrounds, respectively. Predicted myristoylation and palmitoylation sites are indicated with red and blue boxes, respectively. The activation segment is indicated with a green box and the AvrPphB cleavage site with a black arrow.

FIG. 18A-18B. Expression of Pbr1 and Goi2 in additional representative barley lines. PCR amplification from cDNA showing expression and from gDNA showing primer compatibility for FIG. 18A two recombinant inbred lines each from each of the three NAM subpopulations used for GWAS (HR620, HR656, and HR658), exhibiting the parental phenotypes FIG. 18B. Additional lines bringing the total lines tested (excluding RILS) to 12 responding and 12 non-responding when considered with FIG. 12. cDNA was generated from RNA extracted from 10-day old plants, the same age used for AvrPphB response assays. The AvrPphB response for each line is indicated: N, no response; LC, low chlorosis; C, chlorosis; HC, high chlorosis.

FIG. 19A-19B. Nla protease-mediated cleavage of the Arabidopsis PBS1 ‘decoy’ protein in Nicotiana benthamiana. FIG. 19A: the Wheat streak mosaic virus (WSMV) PBS1 decoy protein contains the WSMV Nla protease cleavage motif QYCVYES (SEQ ID NO: 54), while the Soybean mosaic virus (SMV) PBS1 decoy protein contains the SMV Nla protease cleavage motif ESVLSQS (SEQ ID NO: 55). FIG. 19A also discloses “GDKSHVS” as SEQ ID NO: 1. FIG. 19B: the indicated proteins were transiently co-expressed in N. benthamiana leaves and then protein extracted and analyzed by immunoblot. Bands marked with an * indicate cleavage products of PBS1, demonstrating cleavage of the decoy PBS1 proteins only by the matching proteases.

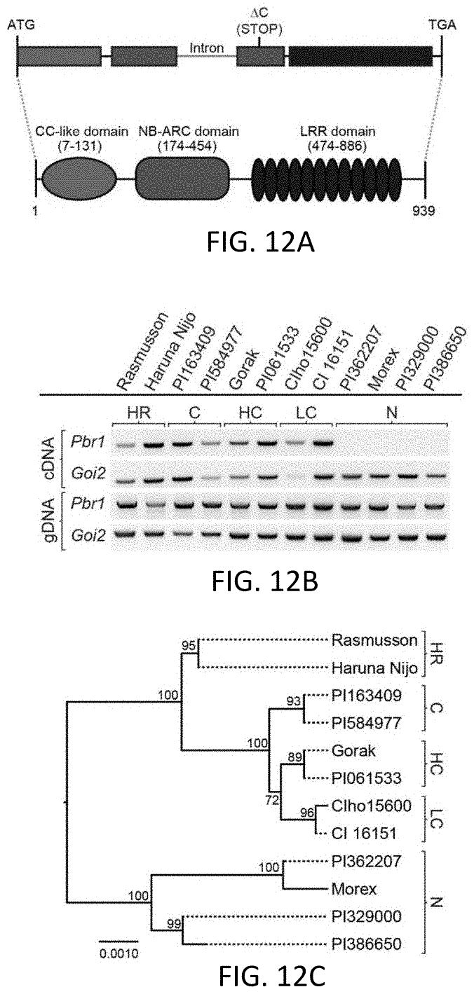

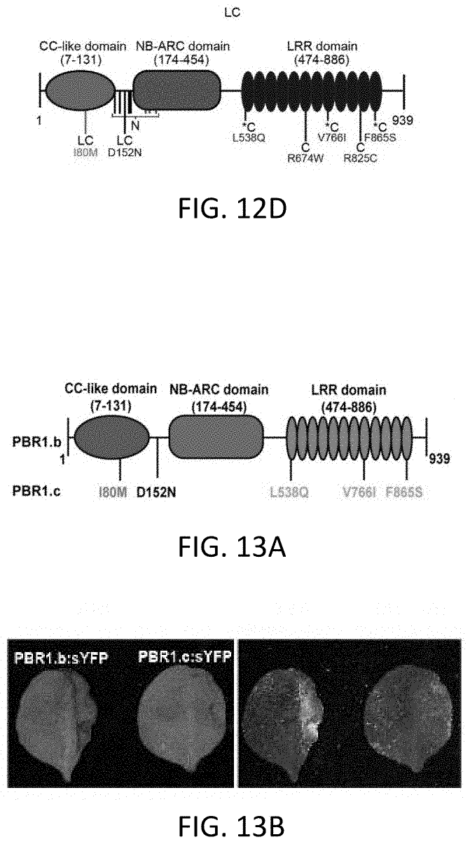

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure. FIG. 12D Schematic illustration of the PBR1.b protein annotated with the approximate location of amino acid substitutions present in the predicted protein products of other Pbr1 alleles and the responses of the corresponding barley lines. *C includes all 3 chlorotic responses.

### DETAILED DESCRIPTION

The compositions, systems and methods now will be described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements.

Likewise, many modifications and other embodiments of the compositions, systems and methods described herein will come to mind to one of skill in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the present disclosure pertains. Moreover, reference to an element by the indefinite article “a” or “an” does not exclude the possibility that more than one element is present, unless the context clearly requires that there be one and only one element. The indefinite article “a” or “an” thus usually includes “at least one.”

Many plant pathogens employ proteases as virulence factors, including bacteria, fungi and viruses. As used herein, “plant pathogen” or “pathogen” means an organism that interferes with or is harmful to plant development and/or growth. Examples of plant pathogens include, but are not limited to, bacteria (e.g., Xanthomonas spp. and Pseudomonas spp.), fungi (e.g., members in the phylum Ascomycetes or Basidiomycetes, and fungal-like organisms including Oomycetes such as Pythium spp. and Phytophthora spp.), insects, nematodes (e.g., soil-transmitted nematodes including Clonorchis spp., Fasciola spp., Heterodera spp., Globodera spp., Opisthorchis spp. and Paragonimus spp.), protozoans (e.g., Phytomonas spp.), and viruses (e.g., Soybean Mosaic Virus (SMV), Turnip Mosaic Virus (TMV), Comovirus spp., Cucumovirus spp., Cytorhabdovirus spp., Luteovirus spp., Nepovirus spp., Potyvirus spp., Tobamovirus spp., Tombusvirus spp. and Tospovirus spp.).

Plants, however, contain innate disease resistance against a majority of plant pathogens. Natural variation for resistance to plant pathogens has been identified by plant breeders and pathologists and can be bred into many plants. These natural disease resistance genes provide high levels of resistance (or immunity) to plant pathogens and represent an economical and environmentally friendly form of plant protection.

Innate disease resistance in plants to plant pathogens typically is governed by the presence of dominant or semidominant resistance (R) genes in the plant and dominant avirulence (avr) genes in the pathogen. In Arabidopsis, an example of this is the dominant R gene RPS5, which mediates recognition of the avrPphB gene from Pseudomonas syringae. Recognition of the AvrPphB protein by the RPS5 protein activates RPS5, which then initiates a disease resistance response that culminates in programmed cell death of cells surrounding the bacteria.

The AvrPphB protein also elicits a cell death response in most varieties of soybean (Glycine max), indicating that these varieties of soybean possess an R gene functionally analogous to RPS5. Soybean contains three genes co-orthologous to PBS1 (GmPBS1a, GmPBS1b, and GmPBS1c). AvrPphB induces cleavage of all three soybean PBS1 proteins, and AvrPphB protease activity is required to activate a cell death response in soybean. These findings indicate that recognition of AvrPphB in soybean likely occurs by the same mechanism as previously described in Arabidopsis.

The present disclosure therefore provides compositions, systems and methods for conferring additional disease resistance to plant pathogens that express specific proteases in plant cells, plant parts or plants by using a modified substrate of a pathogen-specific protease that has a heterologous protease recognition sequence in connection with its corresponding NB-LRR protein.

### Recombinant Nucleic and Amino Acid Molecules

Compositions of the present disclosure include recombinant nucleic and amino acid sequences for modified substrate proteins of pathogen-specific proteases in which an endogenous protease recognition sequence within the substrates are replaced with a heterologous protease recognition sequence.

In one aspect, the present disclosure is directed to a recombinant nucleic acid molecule comprising a nucleotide sequence that encodes at least one substrate protein of a plant pathogen-specific protease and a heterologous pathogen-specific protease recognition sequence within the substrate protein. The substrate protein can be, for example, Glycine max AvrPphB susceptible 1 (GmPBS1). More particularly, the nucleotide sequence encoding at least one substrate protein of a plant pathogen-specific protease and a heterologous pathogen-specific protease recognition sequence within the substrate protein can be one or more of SEQ ID NO:9, SEQ ID NO:11 or SEQ ID NO:13. The nucleotide sequence may encode one or more substrate proteins such as GmPBS1a (SEQ ID NO: 10), GmPBS1b (SEQ ID NO: 12), or GmPBS1c (SEQ ID NO: 14).

PBS1 Homologs in Barley Contain the AvrPphB Recognition Site and are Cleaved by AvrPphB.

The Pseudomonas syringae cysteine protease AvrPphB activates the Arabidopsis resistance protein RPS5 by cleaving a second host protein, PBS1. AvrPphB induces defense responses in other plant species, but the genes and mechanisms mediating AvrPphB recognition in those species have not been previously defined. This disclosure shows that AvrPphB induces defense responses in diverse barley cultivars. Barley contains two PBS1 orthologs. Their products are cleaved by AvrPphB, and barley AvrPphB response maps to a single locus containing a nucleotide-binding leucine-rich repeat (NLR) gene, which is termed AvrPphB Resistance 1 (Pbr1).

Transient co-expression of PBR1 with wild-type AvrPphB, but not a protease inactive mutant, triggered defense responses, indicating that PBR1 detects AvrPphB protease activity. Additionally, PBR1 co-immunoprecipitated with barley and N. benthamiana PBS1 proteins, suggesting mechanistic similarity to detection by RPS5. The disclosed wheat cultivars also recognize AvrPphB protease activity and contain a Pbr1 ortholog. Phylogenetic analyses showed however that Pbr1 is not orthologous to RPS5. The disclosed results indicate that the ability to recognize AvrPphB evolved convergently, and that selection to guard PBS1-like proteins is ancient. Also, the results suggest that PBS1-based decoys are useful to engineer protease effector recognition-based resistance in barley and wheat.

Having found that many barley lines recognize D36E expressing AvrPphB, a question was whether barley contains a recognition system functionally analogous to the Arabidopsis RPS5-PBS1 pathway. Because PBS1 is one of the most well conserved defense genes in flowering plants, with orthologs present in monocot and dicot crop species, an initial question was whether barley contains a PBS1 homolog cleavable by AvrPphB.

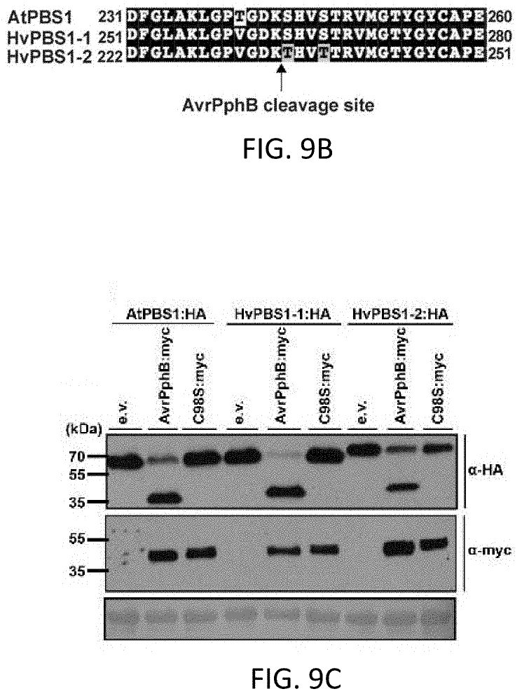

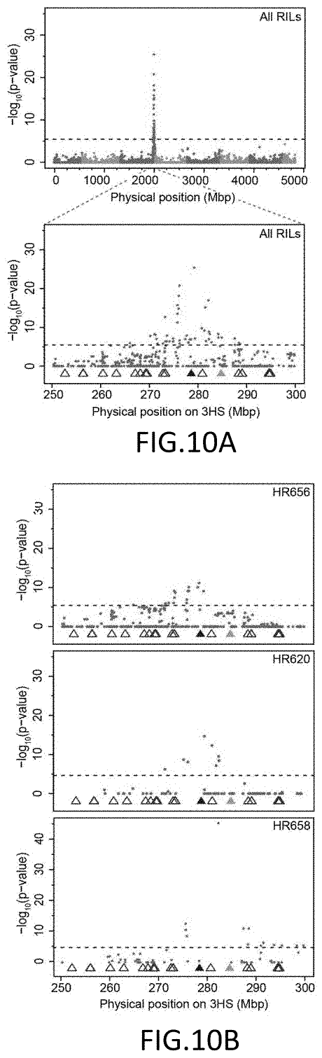

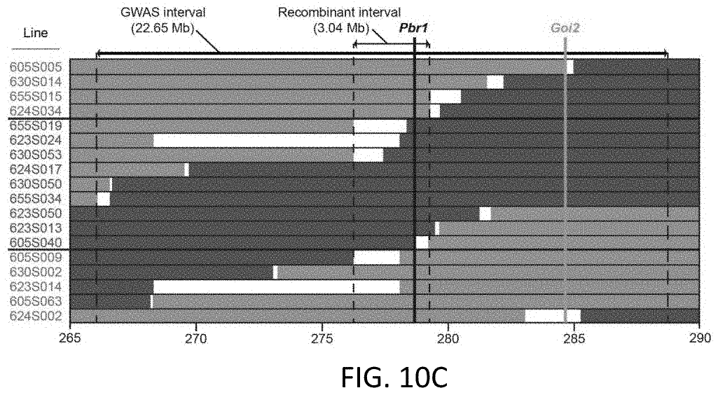

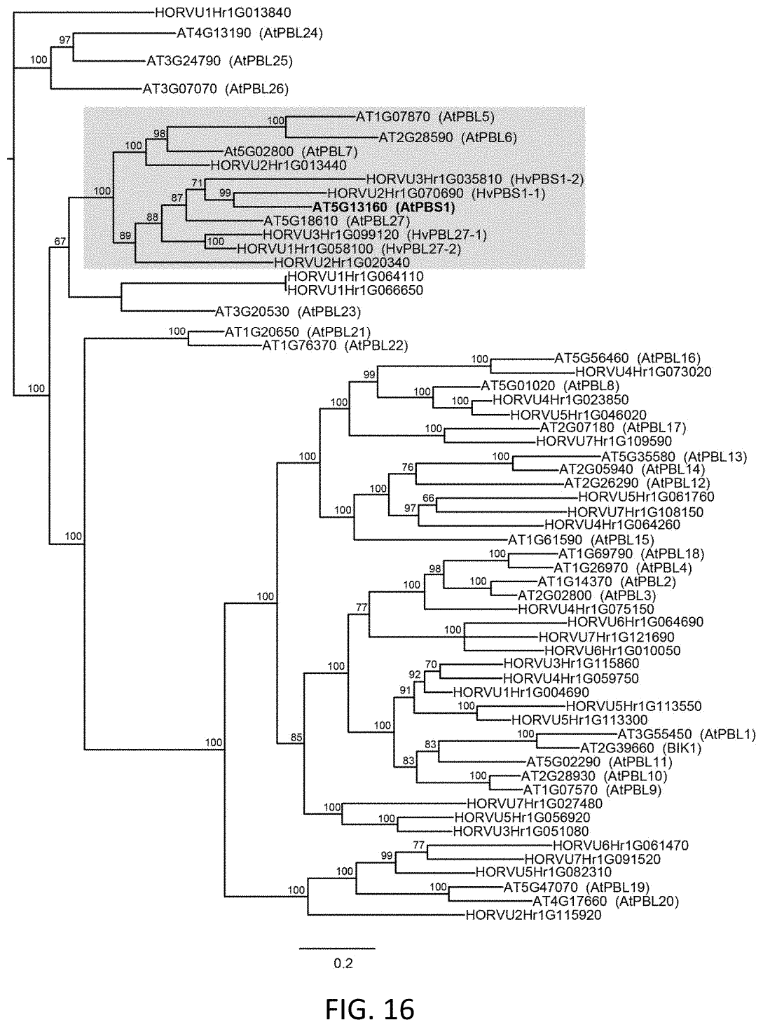

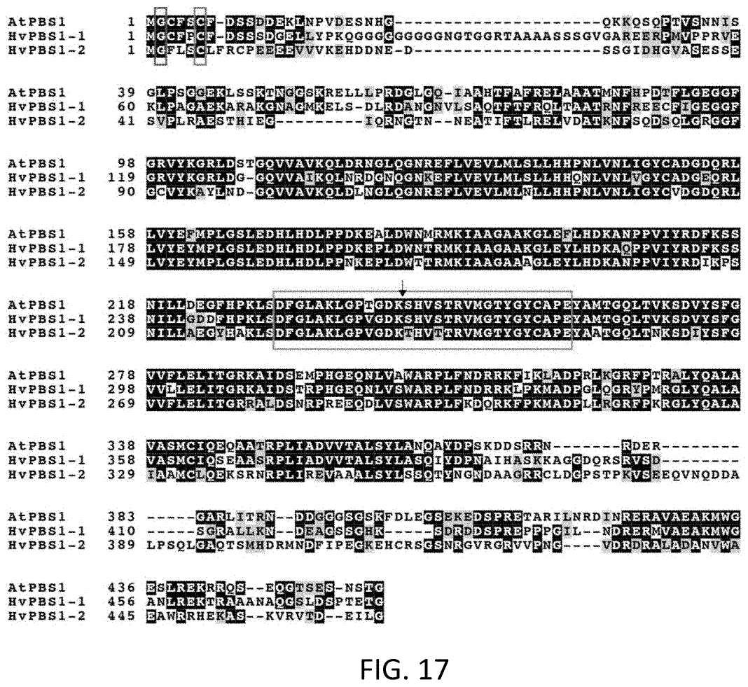

Amino acid sequences were used from all characterized Arabidopsis PBS1-like (AtPBL) proteins, Arabidopsis PBS1 (AtPBS1), and twenty barley PBS1-like (HvPBL) protein sequences homologous to AtPBS1 and AtPBL proteins to identify the barley proteins most closely related to Arabidopsis PBS1. Bayesian phylogenetic analyses showed that HORVU2Hr1G070690.2 (MLOC_13277) was the closest homolog to AtPBS1, whereas HORVU3Hr1G035810.1 (MLOC_12866) was the second most closely related (FIG. 9A; FIG. 16). Both proteins are more similar to AtPBS1 than to other AtPBL and HvPBL proteins, indicating that the two barley genes are co-orthologous to AtPBS1. Full-length amino acid alignments showed that HORVU2Hr1G070690.2 and HORVU3Hr1G035810.1 are 66% and 64% identical to Arabidopsis PBS1, respectively (FIG. 10). Alignment of the two barley gene products and Arabidopsis PBS1 across the kinase domain showed 86% and 79% identity, respectively. Further characterization of the HvPBS1 orthologs showed that each contains several domains that are conserved in AtPBS1, including putative N-terminal palmitoylation and myristoylation sites required for plasma membrane localization and the protease cleavage site sequence recognized by AvrPphB (FIG. 9B; FIG. 17). HORVU2Hr1G070690.2 (MLOC_13277) as HvPbs1-1 and HORVU3Hr1G035810.1 (MLOC_12866) was designated as HvPbs1-2.

Conservation of the AvrPphB cleavage site sequences within the barley PBS1 homologs suggested that AvrPphB would cleave HvPBS1-1 and HvPBS1-2. To test this, HvPBS1-1 and HvPBS1-2 were fused to a three-copy human influenza haemagglutinin (3×HA) epitope tag and transiently co-expressed with AvrPphB:myc in N. benthamiana. Western blot analysis indeed showed that HvPBS1-1:HA and HvPBS1-2:HA are each cleaved by AvrPphB:myc (FIG. 9C). As a control, HvPBS1-1:HA and HvPBS1-2:HA were co-expressed with protease inactive AvrPphB(C98S):myc. This did not produce any cleavage products (FIG. 9C). Collectively, these data show that barley contains two PBS1 homologs whose protein products can be cleaved by AvrPphB and whose function may be analogous to AtPBS1.

A Single NLR Gene-Rich Region in the Barley Genome is Associated with AvrPphB Response.

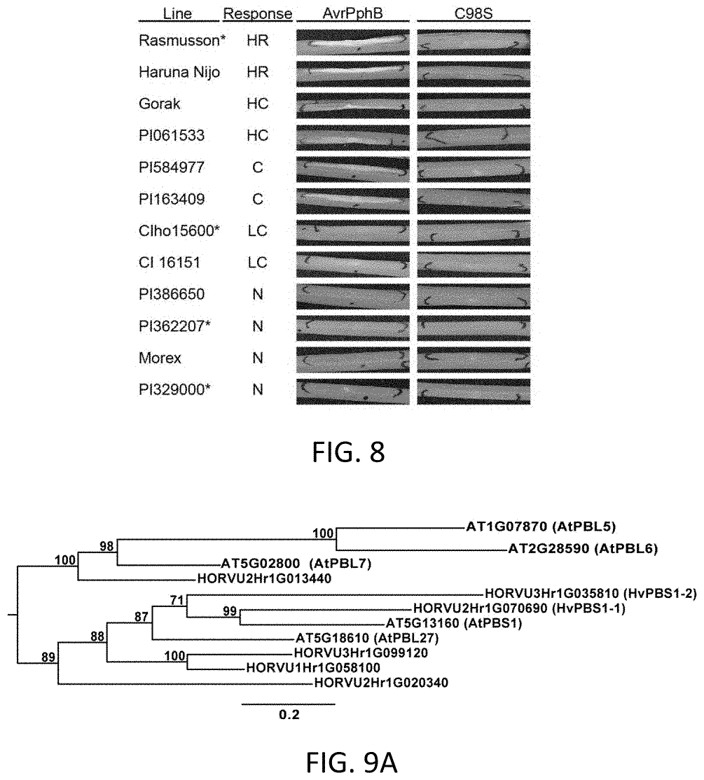

Given the response to AvrPphB in some barley lines and the presence of conserved AvrPphB-cleavable PBS1 homologs in barley, the possibility was that responding barley lines contain a PBS1-guarding NLR analogous to RPS5. To identify candidates, a genome wide association study was carried out (GWAS). The Rasmusson spring barley nested association mapping (NAM) population generated by the US Barley CAP (A. Ollhoff and K. Smith, University of Minnesota, unpublished) contains 6,161 RILs derived from crosses between the elite malting line Rasmusson and 88 diverse donor parents, each of which has associated SNP marker data. Rasmusson, the common parent, displays an HR when infiltrated with D36E expressing AvrPphB whereas the other parents vary in their responses (FIG. 8). For the GWAS, three NAM sub-populations (families) were chosen: two derived from non-responding parents, PI329000 (family HR656) and PI366207 (HR658), and one from a low-chlorosis response parent, CIho15600 (HR620) (FIG. 8).

As expected for a qualitative, single gene trait, the responses segregated ˜1:1 within each family of RILs; 39 of 73 HR656 lines, 19 of 36 HR658 lines, and 29 of 66 HR620 lines displayed an HR following infiltration with D36E expressing AvrPphB (a total of 87 out of 175 RILs tested; Table 3). Co-segregation of AvrPphB response with SNPs was analyzed using the R/NAM package, which included 13,981 SNPs in the analysis of the 175 lines (see Methods). The GWAS identified a 22.65 Mb region between positions 660,376,398 (SNP 3H2_266065765) and 683,030,529 (SNP 3H2_288719896) on the short arm of chromosome 3H associated with AvrPphB response (FIG. 10A). Neither HvPbs1-1 nor HvPbs1-2 are in this region, supporting the hypothesis that an NLR, and not a PBS1 homolog, is the determinant of AvrPphB response. Notably, analysis of each of the three families individually identified the same locus on chromosome 3H as the only significant association (FIG. 10B). The most significant SNP and the number of SNPs used in the analysis varied by population due to the SNP variation between Rasmusson and each of the other parents.

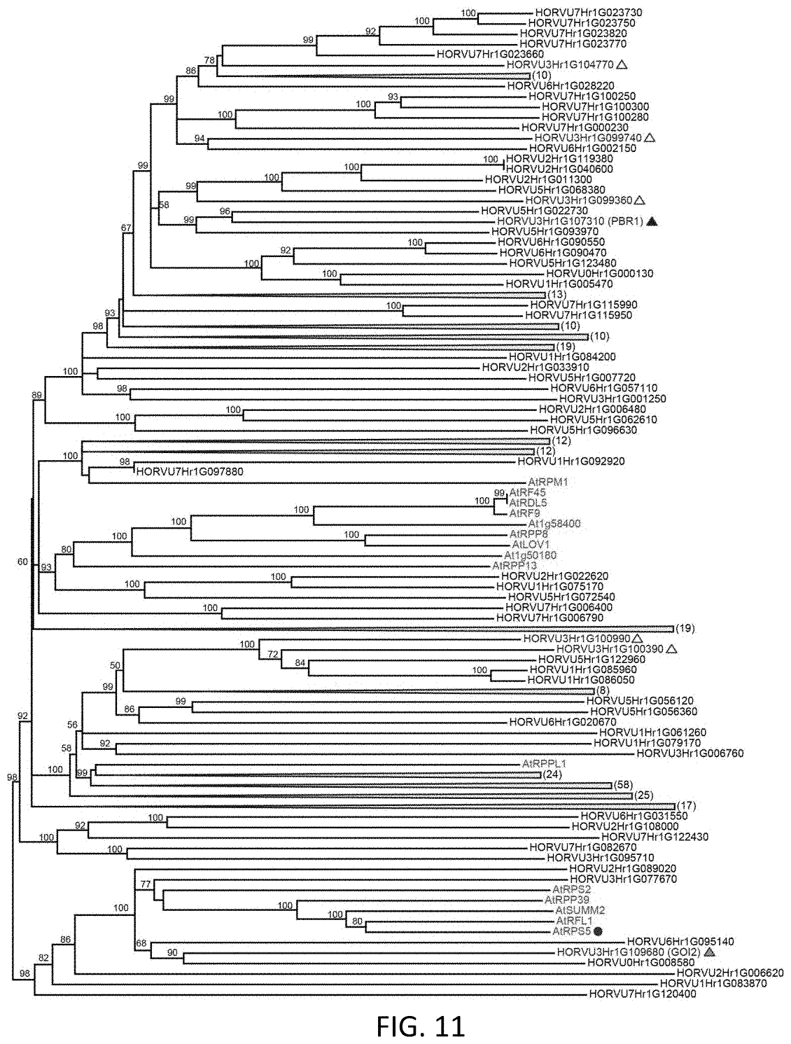

Within the GWAS interval, there are 13 predicted NLR genes, as called by NLR-parser (FIGS. 10A and 10B). In the reference genome, only four encode putative full length NLRs; the rest are fragments, mostly LRR domains and some partial NB-ARC domains. The most significant SNP in the analysis of all lines was S3H2_279293442 (3H:673604075; −log(p)=25.48). The nearest predicted NLR to this SNP, HORVU3Hr1G107310 (3H: 672,928,614-672,932,121), was selected as the top candidate for the determinant of the response to AvrPphB and tentatively named it Pbr1 (AvrPphB Response 1). Despite its association with the most significant SNP, the protein product PBR1 is less closely related to RPS5 in both the encoded CC (FIG. 11A) and NB-ARC (FIG. 11B) domains than the protein products of the other NLR genes within the 22.65 Mb region. Of these, the one encoding the NLR most closely related to RPS5, HORVU3Hr1G109680 (3H: 679064240-679072712), on the edge of the GWAS interval, was selected as an additional gene of interest and referred to it hereafter as Goi2. PBR1 and RPS5 are 17% identical to each other at the CC domain and 29% at the NB-ARC domain, while GOI2 and RPS5 are 24% and 45% identical to each other at those domains, respectively. For comparison, PBR1 and GOI2 are 23% identical to each other at the CC domain and 20% at the NB-ARC domain.

As a next step to identify the determinant of the AvrPphB response, SNP data was used for the entire NAM population to find additional recombinants within the 22.65 Mb GWAS interval. Based on haplotype data within the region, eighteen apparent recombinants were selected from four additional families with non-AvrPphB-responding parents and phenotyped (Table 3). Adding the genotype and phenotype data of these new lines to the GWAS increased the significance of many of the SNPs, but did not narrow the interval. However, using the estimated recombination breakpoints and the phenotypes of the individual RILs to fine map the determinant of the response resulted in a 3.04 Mb region within the GWAS peak that contains Pbr1 and no other NLR gene (FIG. 10C), supporting Pbr1 rather than Goi2 as the candidate determinant.

AvrPphB, and not a Catalytically Inactive Derivative, Triggers Defense Responses in Barley.

Of the 150 barley genotypes screened, 29 were scored as LC, 17 as C, 13 as HC and 6 as HR (Table 2). Both chlorotic and cell death responses were considered defense responses, as both have been documented as such for grasses.

To test whether barley can detect AvrPphB protease activity, AvrPphB was delivered to barley leaves using P. syringae pathovar tomato strain D36E, which is a derivative of strain DC3000 lacking all type III secretion system effectors. Seedlings were infiltrated with D36E expressing AvrPphB or the catalytically inactive mutant AvrPphB(C98S) and scored for visible responses at 2 and 5 days post infiltration.

A diverse set of barley lines were tested and a variety of responses were observed. Representative examples are shown in FIG. 8, and the complete list of cultivars and their responses are provided in Table 2. Based on the range of responses, the phenotypes were scored as no response (N) or one of 4 responses: low chlorosis (LC) indicates a weak, but noticeable response, chlorosis (C) for strong yellow, high chlorosis (HC) for a chlorotic response that gives way to cell death, and hypersensitive reaction (HR) for cell collapse and browning visible by day 2.

Pbr1 is Expressed in Lines Responding to AvrPphB and Allelic Variation Correlates with Phenotype.

The reference genome used in the GWAS is from the barley line Morex, an AvrPphB-non-responding line (FIG. 8). Therefore, the reference genome is likely to have a nonfunctional copy of, or lack completely, the NLR hypothesized to detect activity of AvrPphB. In the Morex genome, Pbr1 is annotated as containing just a truncated NB-ARC domain and LRR domain, missing an N-terminal domain. In contrast, Goi2 encodes a full length NLR (965 aa) with an RPS5-like CC domain (aa 27-66), NB-ARC domain (aa 156-439), and LRR (aa 537-864). To see if either gene sequence varies in the responding line Rasmusson, Pbr1 and Goi2 from that line were sequenced. The Rasmusson allele of Goi2 is highly similar to the Morex allele, with only 3 nonsynonymous mutations between them (N860I, R808H, and V282L). Among the differences between the two Pbr1 sequences, a single nucleotide insertion in Rasmusson was found that restores a larger open reading frame (FIG. 12), resulting in a predicted full-length NLR (939 aa) with an intact CC_{EDVID} (“EDVID” disclosed as SEQ ID NO: 38) domain (aa 7-131), NB-ARC domain (aa 174-454), and an LRR domain containing 12 repeats (aa 474-886). For Pbr1, refer to the allele in Morex as Pbr1.a (GenBank: MH5956) and in Rasmusson as Pbr1.b (GenBank: MH595618).

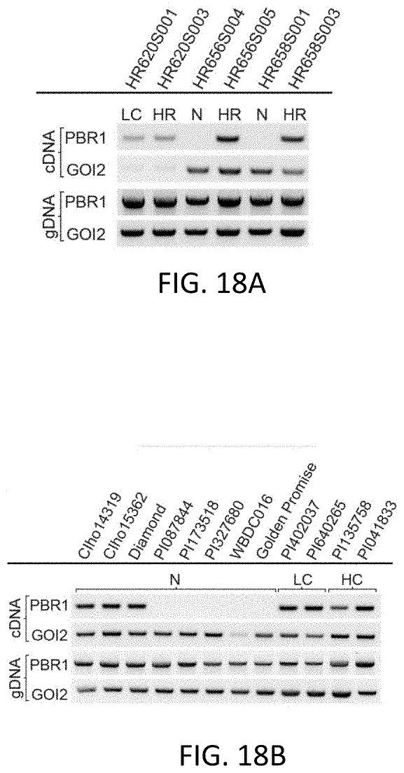

Reverse transcriptase PCR (RT-PCR) was used to test the expression of Pbr1 alleles in Morex and Rasmusson, as well as a variety of other barley lines ranging in AvrPphB-induced responses. Pbr1 was expressed in lines that respond to AvrPphB either with HR or chlorosis (Rasmusson, Haruna Nijo, PI061533, Gorak, PI584977, PI163409, CIho15600, and CI 16151), but not in non-responding lines (PI329000, PI386650, PI362207, and Morex) (FIG. 12B). The primers used for RT-PCR were compatible with all genotypes tested, as shown by amplification from genomic DNA, and spanned an intron to differentiate cDNA from any genomic DNA contamination. 30 total lines were investigated: 12 responders, 12 non-responders, and 6 RILS from the 3 NAM subpopulations used for GWAS. Pbr1 was expressed in all responding lines, but not expressed in 9 out of 12 non-responding lines (FIG. 12B and FIG. 18). For comparison, Goi2 expression was assayed in these lines as well and varying levels of expression were found that did not correspond to AvrPphB response (FIG. 12B).

Because point mutations within an NLR can lead to changes in observable HR in planta, to see if the responses to AvrPphB observed across different barley lines corresponded with sequence polymorphism at Pbr1, Pbr1 alleles of 10 additional barley lines selected at random from among the different response phenotypes (1 HR, 2 HC, 2 C, 2 LC, and 3 non-responding lines) were sequenced and compared to Pbr1.a and Pbr1.b from Morex and Rasmusson, respectively. The nucleotide sequences cluster by phenotype (FIG. 12C) (SEQ ID NOS 26-37) when analyzed from start codon to stop codon using the Neighbor-Joining method. The non-responding lines, PI329000, PI386650, PI362207, and Morex have unique but similar alleles. The HR line Haruna Nijo, like Rasmusson, has the Pbr1.b allele. The amino acid sequences of Pbr1 in the two lines each from the three chlorosis response groups (LC lines CI 16151 and CIho15600, C lines PI584977 and PI163409, and HC lines Gorak and PI061533) are identical within and different across the groups; all contain 3 common substitutions compared to the Rasmusson allele Pbr1.b, including an L538Q substitution in the LRR. Together these observations suggest that sequence polymorphism in Pbr1 determines response to AvrPphB.

The Product of Pbr1 Allele Pbr1.c Recognizes AvrPphB Protease Activity in N. benthamiana.

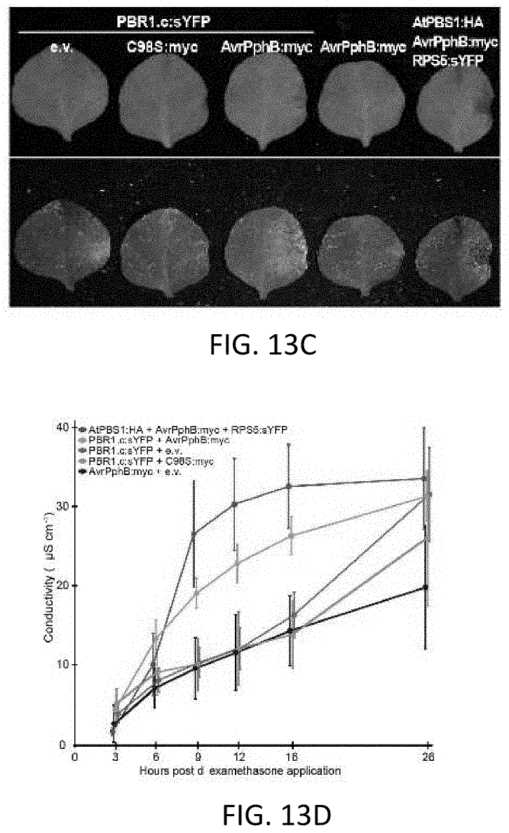

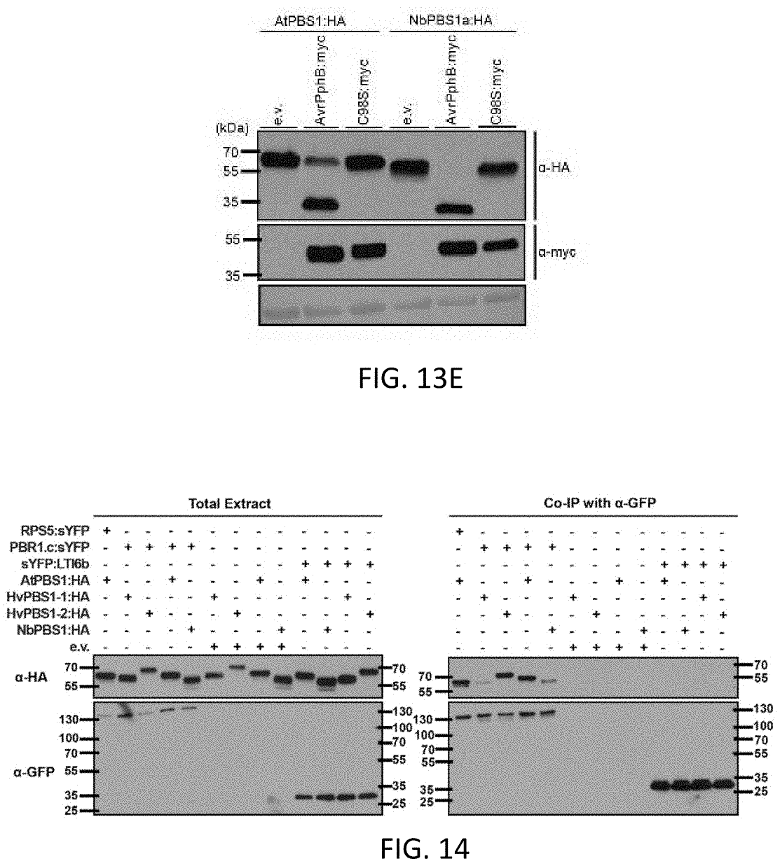

To directly test whether PBR1 mediates recognition of AvrPphB, transient expression assay was developed in N. benthamiana. Pbr1.b from cultivar Rasmusson was cloned into a dexamethasone-inducible vector along with a C-terminal fusion to super yellow fluorescent protein (PBR1.b:sYFP). Unfortunately, transient expression of PBR1.b:sYFP alone resulted in HR with complete tissue collapse within 24 hours of transgene induction (FIG. 13B), indicating that PBR1.b is auto-active when overexpressed in N. benthamiana.

To circumvent the problem posed by auto-activity of the PBR1.b protein, a Pbr1 allele from the LC line CI 16151 was tested (FIG. 8). This allele was designed Pbr1.c (GenBank: MH595619). PBR1.b and PBR1.c differ by five amino acid substitutions, of which three are located within the leucine-rich repeat domain (FIG. 13A). Transient expression of a PBR1.c:sYFP fusion protein in the absence of AvrPphB consistently produced a weaker HR than PBR1.b (FIG. 13B). This result allowed testing whether the HR was enhanced in the presence of active AvrPphB.

PBR1.c:sYFP and AvrPphB:myc were transiently co-expressed in N. benthamiana and assessed cell death. As a control, AtPBS1:HA and RPS5:sYFP with AvrPphB:myc, a combination that activates cell death in N. benthamiana were co-expressed. Transient co-expression of PBR1.c:sYFP with AvrPphB:myc resulted in observable tissue collapse 24 hours post-transgene induction, whereas co-expression of PBR1.c:sYFP with either empty vector (e.v.) or AvrPphB(C98S):myc resulted in a much weaker cell death response (FIG. 13C). Further, transient expression of AvrPphB:myc in the absence of PBR1.c:sYFP did not trigger HR, indicating that the cell death response requires PBR1.c (FIG. 13C). An electrolyte leakage analysis was performed to better quantify PBR1.c-mediated cell death. Transient co-expression of PBR1.c:sYFP with AvrPphB-myc induced greater ion leakage than PBR1.c:sYFP co-expressed with either empty vector or AvrPphB(C98S):myc between 9 and 16 hours after transgene induction, confirming that PBR1.c:sYFP recognizes and mediates a response to AvrPphB protease activity (FIG. 13D). By 26 hours post transgene induction, PBR1.c:sYFP expressed with AvrPphB(C98S) or empty vector induced ion leakage similar to that observed with co-expression of PBR1.c:sYFP and wild-type AvrPphB, indicating that PBR1.c:sYFP is weakly auto-active, consistent with the HR assays (FIG. 13D).

The observation that AvrPphB, but not AvrPphB(C98S), activates PBR1.c-mediated cell death in N. benthamiana even in the absence of a barley PBS1 protein suggested that AvrPphB might be cleaving an N. benthamiana ortholog of PBS1 and that PBR1.c is recognizing that cleavage. Using a reciprocal BLAST and the amino acid sequence of Arabidopsis PBS1, an ortholog of PBS1 was identified in the N. benthamiana genome, Niben101Scf02996g03008.1, and designated it NbPBS1. Importantly, NbPBS1 contains the AvrPphB cleavage site sequence and is thus predicted to be cleaved by AvrPphB. To determine whether in the transient assay PBR1.c is guarding an endogenous PBS1 ortholog, NbPBS1:HA was co-expressed with either AvrPphB:myc or AvrPphB(C98S):myc. Co-expression with AvrPphB:myc, but not the protease inactive mutant, resulted in cleavage of NbPBS1:HA within 6 hours post-transgene expression, showing that NbPBS1:HA is a substrate for AvrPphB (FIG. 13E) and that its cleavage could be the trigger for PBR1.c.

PBS1 Proteins Immunoprecipitate with Barley PBR1.c when Transiently Co-Expressed in N. benthamiana.

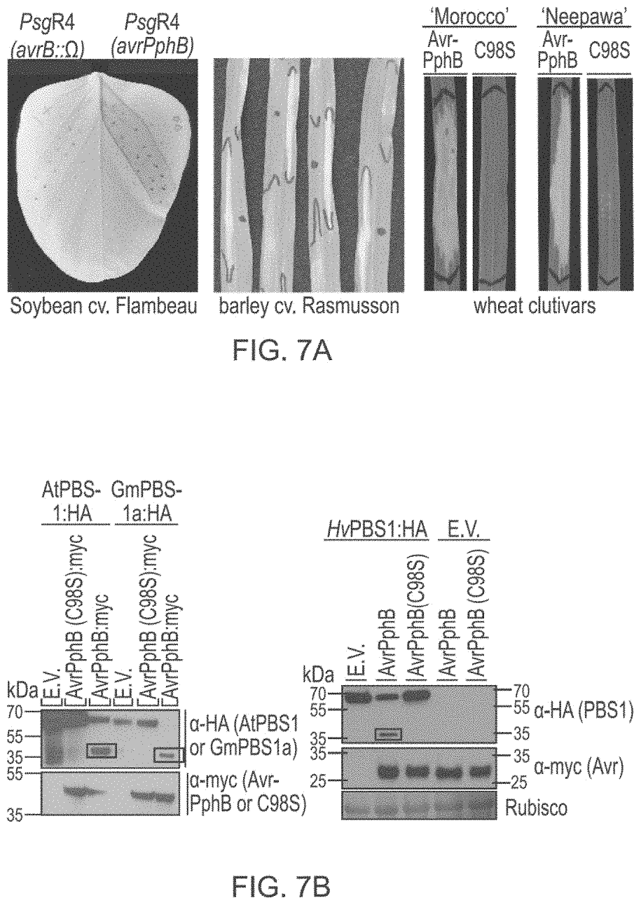

To determine whether PBR1.c is activated by sensing cleavage of PBS1 proteins, co-immunoprecipitation (co-IP) analyses of PBR1.c with HvPBS1-1:HA, HvPBS1-2:HA, AtPBS1:HA, or NbPBS1:HA was performed. As a positive control, AtPBS1:HA was co-expressed with RPS5:sYFP, which forms a pre-activation complex in the absence of AvrPphB. As a negative control, the plasma membrane-localized fusion protein sYFP:LTI6b with each of the PBS1 proteins (Cutler et al., 2000). Consistent with the hypothesis being tested, HvPBS1-1:HA, HvPBS1-2:HA, and AtPBS1:HA immunoprecipitated with PBR1.c:sYFP and not with sYFP:LTI6b, demonstrating that PBR1.c forms a complex with PBS1 proteins from barley and Arabidopsis in the absence of AvrPphB (FIG. 14). NbPBS1:HA also immunoprecipitated with PBR1.c:sYFP (and not with sYFP:LTI6b) supporting the notion that AvrPphB-mediated cleavage of NbPBS1 activates PBR1.c-dependent HR in N. benthamiana. Though all of the PBS1 proteins immunoprecipitated with PBR1.c:sYFP, PBR1.c:sYFP preferentially interacted with HvPBS1-2:HA and AtPBS1:HA (FIG. 7). Collectively, these data suggest that PBR1 forms a pre-activation complex with one or more barley PBS1 orthologs, providing further evidence that PBR1 is the guard that recognizes AvrPphB activity. Importantly, CSS-PALM 4.0 (http://csspalm.biocuckoo.org/) predicts that PBR1.b and PBR1.c are palmitoylated at Cys314, suggesting co-localization with AvrPphB and barley PBS1 orthologs at the plasma membrane.

Wheat (Triticum aestivum Subsp. Aestivum) Also Recognizes AvrPphB Protease Activity.

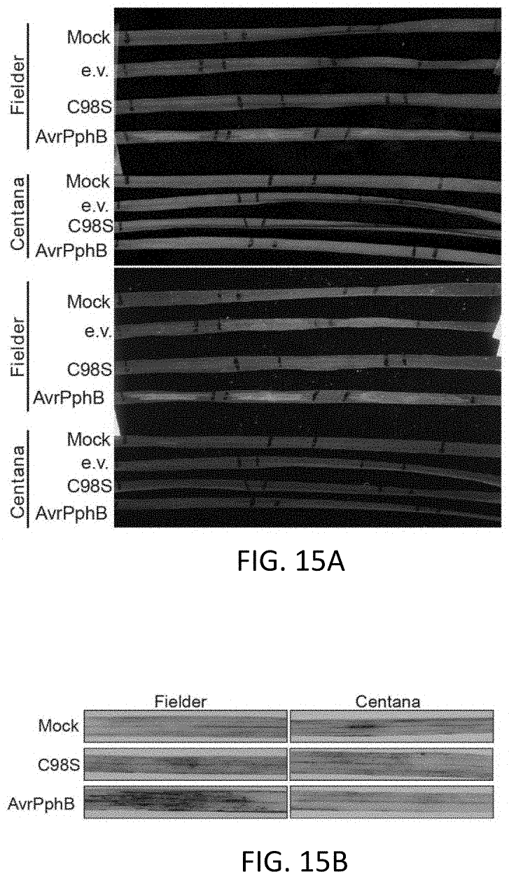

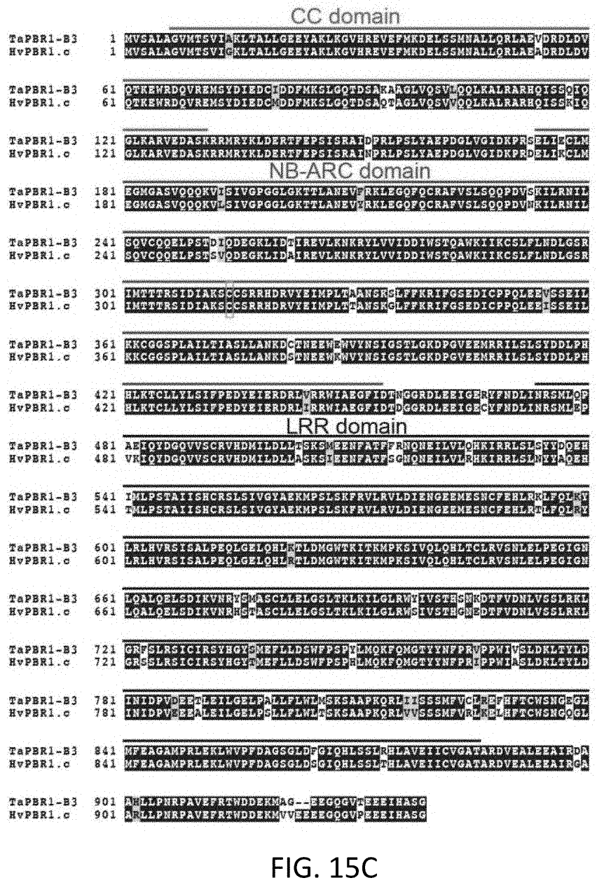

Recently an ortholog of Arabidopsis PBS1 was identified in wheat, TaPBS1, that localizes to the plasma membrane when transiently expressed in N. benthamiana and is cleaved by AvrPphB. However, it remained unclear whether wheat recognizes AvrPphB protease activity and would thus likely contain a functional analog of RPS5, such as Pbr1. 34 wheat varieties obtained from the U.S. Department of Agriculture Wheat Germplasm Collection were screened for their response to D36E expressing AvrPphB (FIG. 15A; Table 4). Twenty-nine responded with chlorosis, while five showed no visible response by three days post-inoculation (FIG. 15A; Table 3). No line responded to the protease inactive mutant AvrPphB(C98S).

To further characterize the chlorotic response in wheat, 3,3′-diaminobenzidine (DAB) staining was used to examine hydrogen peroxide accumulation following leaf infiltration with D36E expressing either AvrPphB or AvrPphB(C98S). Consistent with the chlorotic phenotype, wheat cv. Fielder accumulated detectable hydrogen peroxide within the infiltrated area when inoculated with D36E expressing AvrPphB, whereas the mock and AvrPphB(C98S) treatments resulted in minimal hydrogen peroxide accumulation (FIG. 15B). In contrast, there was no significant hydrogen peroxide accumulation in wheat cv. Centana inoculated with either strain (or mock), consistent with the lack of chlorotic response of this line to AvrPphB. The correlation of chlorosis and hydrogen peroxide accumulation specifically in response to active AvrPphB is consistent with recognition in wheat associated with defense.

To examine whether this recognition in wheat might be mediated by a Pbr1 ortholog, the T. aestivum subsp. aestivum genome was searched using the Ensembl genome browser (release TGACv1). TRIAE_CS42_3B_TGACv1_226949_AA0820360 was found to be an ortholog, and was designated TaPbr1. TaPbr1 is located on wheat chromosome 3B in a position syntenic with barley Pbr1 and encodes an NLR consisting of a predicted Rx-like coiled-coil domain (aa 7-131), a nucleotide-binding domain (aa 174-454), and a leucine-rich repeat domain (aa 474-895) (FIG. 15C). Full-length amino acid sequence alignment of barley PBR1.c and TaPBR1 shows 93% amino acid identity (FIG. 15C). Further, TaPBR1, like barley PBR1, is predicted to be palmitoylated at Cys314, suggesting co-localization with AvrPphB and wheat PBS1. It thus seems likely that TaPBR1 functions as the cognate NLR protein that mediates recognition of AvrPphB protease activity in wheat.

The PBS1 gene is highly conserved across all angiosperms, including barley, wheat, rice and corn. AvrPphB protease from Pseudomonas syringae cleaves PBS1 proteins from all crops tested, including barley and soybean. Furthermore, AvrPphB protease activity induces resistance in barley, wheat and soybean. Protease from rust fungi that infect soybean or wheat (different fungal species) are disclosed herein.

### Discussion

Recognition of the P. syringae AvrPphB protease by the Arabidopsis RPS5 NLR protein is a well characterized example of indirect effector recognition. Though AvrPphB is recognized by other plant species such as soybean and common bean, the disease resistance genes responsible for recognition outside of Arabidopsis have not been cloned, and the underlying molecular mechanisms are unknown. The evidence herein supports the conclusion that barley and Arabidopsis have convergently evolved NLRs able to detect effectors that structurally modify PBS1-like kinases: barley cultivars respond to AvrPphB but not to a protease inactive mutant of AvrPphB, barley contains an NLR gene evolutionarily distinct from RPS5 that mediates a strong HR when co-expressed with avrPphB in N. benthamiana, and AvrPphB associates with and cleaves PBS1 orthologs from monocots and dicots.

While AvrPphB is not known to be present in any pathogens of barley, it is a member of a family of proteases present in many phytopathogenic bacteria. More generally, proteases that target host proteins are found in many, diverse types of pathogens, and conserved kinases that are involved in PTI are expected to be common effector targets. Though the functional roles of HvPBS1-1 and HvPBS1-2 as well as other barley PBS1-like proteins are unknown, given their conservation in many flowering plant families, they may have a role in PTI signaling as observed in Arabidopsis. Results herein however, support that barley deploys an effector protease recognition mechanism similar to that of recognition of AvrPphB by Arabidopsis RPS5, wherein barley PBR1 guards RLCKs such as HvPBS1-1 and HvPBS1-2 such that it is activated upon their cleavage. Within Arabidopsis populations, RPS5 is maintained as a balanced presence/absence polymorphism despite inconsistent interaction with Pseudomonas strains expressing AvrPphB homologs, suggesting other effectors are also imposing selection pressure. How many and which effectors from barley pathogens target RLCKs is unknown.

The convergent evolution of the shared ability of PBR1 and RPS5 to recognize AvrPphB aligns with the prediction that RLCKs that function in plant immunity are common targets of pathogen effectors and that selection to guard these proteins is ancient and widespread. A similar example of convergent evolution of NLR specificity has been described for the RPM1 and Rpg1b/Rpg1r proteins of Arabidopsis and soybean, all three of which detect effector induced modifications of RIN4 proteins. Like PBL proteins, RIN4 is targeted by multiple effectors, consistent with these proteins serving critical functions in plant immunity. It is especially interesting that PBR1 and RPS5 independently evolved to detect PBL cleavage instead of directly interacting with AvrPphB or integrating a PBL decoy. Direct interaction limits the number of effectors a single NLR can detect, while guarding a commonly targeted host protein expands the response spectrum, thus allowing the NLR to detect multiple pathogen effectors. The guarding strategy might impose purifying selection on RLCKs themselves or selection to integrate an RLCK decoy into an NLR: either would reduce the risk of any guard-guardee genetic mismatch that might lead to hybrid necrosis. However, there is no obvious reason why PBR1 and RPS5 would both have each evolved to guard PBS1, rather than distinct avrPphB substrates, and data disclosed do not rule out the possibility that in barley PBR1 is activated by cleavage of one or more different RLCKs.

When assessing the functional role of PBR1.c in AvrPphB recognition, it was found its co-expression with AvrPphB elicited cell death in N. benthamiana even in the absence of barley PBS1 expression. Given that PBR1.c does not contain the AvrPphB cleavage site sequence, it is likely PBR1.c is sensing AvrPphB-mediated cleavage of an endogenous N. benthamiana PBL protein. PBS1 ortholog NbPBS1 indeed associates with PBR1 in the absence of AvrPphB and is cleaved by AvrPphB. This was true also of AtPBS1 and the barley PBS orthologs HvPBS1-1 and HvPBS1-2. Taken together, these data strongly suggest that in barley, PBR1.c detects AvrPphB protease activity by sensing cleavage of a PBS1 protein, analogous to AvrPphB-detection by RPS5.

Pbr1 is expressed in the 12 tested barley lines that respond to AvrPphB, and in only 3 of 12 lines that do not respond. The sequence polymorphisms found in Pbr1 alleles across the 12 responding barley lines correlate with the presence and severity of the AvrPphB response (i.e. chlorosis versus strong HR). These data support that mutations within the Pbr1 coding sequence impact the macroscopic phenotype observed when AvrPphB is present. Mutagenesis screens of specific NLRs have been shown to modify the severity of phenotype and specificity of interaction. Natural examples of the effect of single or few mutations impacting NLR function include the Pi-ta NLR in Oryzae spp., in which a single amino acid is highly correlated to resistance, and the barley Mla locus, which encodes alleles with over 90% amino acid sequence identity that recognize different effector proteins. In wheat, alleles of the Pm3 gene have very little sequence diversity, but just two amino acid mutations expand the effector recognition capacity of Pm3f and increase its activity. The polymorphisms in PBR1 will be characterized to determine which, if any, modify the response to AvrPphB response or if any impact specificity. However, the difference in auto-activity between PBR1.b and PBR1.c when expressed in N. benthamiana is further evidence that the allelic sequence polymorphism contributes to phenotype.

The evidence that PBR1 is activated by cleavage of a PBS1 or PBS1-like protein supports that PBS1-based decoys can be used to expand protease effector recognition in barley. Barley powdery mildew (Blumeria graminis f. sp. hordei; Bgh) and Wheat streak mosaic virus (WSMV) are two barley pathogens known to deploy proteases as part of the infection process. BEC1019 is a putative metalloprotease secreted by Bgh and is conserved among ascomycete fungi. Notably, silencing of BEC1019 by both Barley stripe mosaic virus- and single cell RNAi-based methods reduces Bgh virulence, suggesting BEC1019 is required for Bgh pathogenicity. Similar to other Potyviruses, WSMV expresses a protease, designated the nuclear inclusion antigen (NIa), that is essential for viral replication and for proper temporal expression of potyviral genes in planta. The cleavage site sequence recognized by the NIa protease has been identified. Insertion of the BEC1019 or NIa protease cleavage site sequence into the barley PBS1 proteins should enable recognition of these proteases by PBR1. This approach could also be extended into wheat given that PBR1 and PBS1 are conserved.

### Definitions

As used herein, a “nucleic acid” sequence means a DNA or RNA sequence. The term encompasses sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

As used herein, “recombinant,” when used in connection with a nucleic acid molecule, means a molecule that has been created or modified through deliberate human intervention such as by genetic engineering. For example, a recombinant nucleic acid molecule is one having a nucleotide sequence that has been modified to include an artificial nucleotide sequence or to include some other nucleotide sequence that is not present within its native (non-recombinant) form.

Further, a recombinant nucleic acid molecule has a structure that is not identical to that of any naturally occurring nucleic acid molecule or to that of any fragment of a naturally occurring genomic nucleic acid molecule spanning more than one gene. A recombinant nucleic acid molecule also includes, without limitation, (a) a nucleic acid molecule having a sequence of a naturally occurring genomic or extrachromosomal nucleic acid molecule, but which is not flanked by the coding sequences that flank the sequence in its natural position; (b) a nucleic acid molecule incorporated into a construct, expression cassette or vector, or into a host cell's genome such that the resulting polynucleotide is not identical to any naturally occurring vector or genomic DNA; (c) a separate nucleic acid molecule such as a cDNA, a genomic fragment, a fragment produced by polymerase chain reaction (PCR) or a restriction fragment; and (d) a recombinant nucleic acid molecule having a nucleotide sequence that is part of a hybrid gene (i.e., a gene encoding a fusion protein). As such, a recombinant nucleic acid molecule can be modified (chemically or enzymatically) or unmodified DNA or RNA, whether fully or partially single-stranded or double-stranded or even triple-stranded.

A nucleic acid molecule (or its complement) that can hybridize to any of the uninterrupted nucleotide sequences described herein, under either highly stringent or moderately stringent hybridization conditions, also is within the scope of the present disclosure.

As used herein, “stringent conditions” means conditions under which one nucleic acid molecule will hybridize to its target to a detectably greater degree than to other sequences (e.g., at least two-fold over background). Stringent conditions can be sequence-dependent and will be different in different circumstances. By controlling the stringency of the hybridization and/or washing conditions, target sequences that are 100% complementary to the nucleic acid molecule can be identified (i.e., homologous probing). Alternatively, the stringent condition can be adjusted to allow some mismatching in sequences so that lower degrees of similarity are detected (i.e., heterologous probing).

Typically, stringent conditions can be one in which the salt concentration is less than about 1.5 M Na⁺, typically about 0.01 M to 1.0 M Na⁺ (or other salts) at about pH 7.0 to 8.3, and a temperature of at least 30° C. for short molecules (e.g., 10 to 50 nucleotides) and of at least 60° C. for long molecules (e.g., greater than 50 nucleotides). Stringent conditions also can be achieved by adding destabilizing agents such as formamide.

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

An exemplary low stringent condition includes hybridizing with a buffer solution of about 30% to about 35% formamide, 1 M NaCl, 1% SDS (sodium dodecyl sulphate) at about 37° C., and washing in about 1× to 2×SSC (20×SSC=3.0 M NaCl/0.3 M trisodium citrate) at about 50° C. to about 55° C. Wash buffers optionally can comprise about 0.1% to about 1% SDS.

An exemplary moderate stringent condition includes hybridizing in about 40% to about 45% formamide, 1.0 M NaCl, 1% SDS at about 37° C., and washing in about 0.5× to 1×SSC at about 55° C. to about 60° C. Wash buffers optionally can comprise about 0.1% to about 1% SDS.

An exemplary high stringent condition includes hybridizing in about 50% formamide, 1 M NaCl, 1% SDS at about 37° C., and washing in about 0.1×SSC at about 60° C. to about 65° C. Wash buffers optionally can comprise about 0.1% to about 1% SDS.

The duration of hybridizing generally can be less than 24 hours, usually about 4 hours to about 12 hours. The duration of the washing can be at least a length of time sufficient to reach equilibrium. Additional guidance regarding such conditions is readily available in the art, for example, in Molecular Cloning: A Laboratory Manual, 3rd ed. (Sambrook & Russell eds., Cold Spring Harbor Press 2001); and Current Protocols in Molecular Biology (Ausubel et al. eds., John Wiley & Sons 1995).

An example of a recombinant nucleic acid molecule encoding a modified substrate protein of a pathogen-specific protease therefore includes a nucleotide sequence that encodes PBS1 in which its endogenous AvrPphB cleavage site (SEQ ID NO:1) is replaced with a heterologous AvrRpt2 cleavage site (SEQ ID NO:2).

Methods for synthesizing nucleic acid molecules are well known in the art, such as cloning and digestion of the appropriate sequences, as well as direct chemical synthesis (e.g., ink-jet deposition and electrochemical synthesis). Methods of cloning nucleic acid molecules are described, for example, in Ausubel et al. (1995), supra; Copeland et al. (2001) Nat. Rev. Genet. 2:769-779; PCR Cloning Protocols, 2nd ed. (Chen & Janes eds., Humana Press 2002); and Sambrook & Russell (2001), supra. Methods of direct chemical synthesis of nucleic acid molecules include, but are not limited to, the phosphotriester methods of Reese (1978) Tetrahedron 34:3143-3179 and Narang et al. (1979) Methods Enzymol. 68:90-98; the phosphodiester method of Brown et al. (1979) Methods Enzymol. 68:109-151; the diethylphosphoramidate method of Beaucage et al. (1981) Tetrahedron Lett. 22:1859-1862; and the solid support methods of Fodor et al. (1991) Science 251:767-773; Pease et al. (1994) Proc. Natl. Acad. Sci. USA 91:5022-5026; and Singh-Gasson et al. (1999) Nature Biotechnol. 17:974-978; as well as U.S. Pat. No. 4,485,066. See also, Peattie (1979) Proc. Natl. Acad. Sci. USA 76:1760-1764; as well as EP Patent No. 1 721 908; Int'l Patent Application Publication Nos. WO 2004/022770 and WO 2005/082923; US Patent Application Publication No. 2009/0062521; and U.S. Pat. Nos. 6,521,427; 6,818,395 and 7,521,178.

In addition to the full-length nucleotide sequence of a nucleic acid molecule encoding a modified substrate/fusion protein, it is intended that the nucleic acid molecule can be a fragment or variant thereof that is capable of functioning as a substrate. For nucleotide sequences, “fragment” means a portion of a nucleotide sequence of a nucleic acid molecule, for example, a portion of the nucleotide sequence encoding a modified substrate protein. Fragments of a nucleotide sequence may retain the biological activity of the reference nucleic acid molecule. For example, less than the entire sequence disclosed in SEQ ID NO:10 can be used and will encode a modified substrate protein that interacts with a pathogen-specific protease and that retains its ability to interact with its corresponding NB-LRR protein. Likewise, a fragment of a nucleotide sequence encoding the modified substrate protein can be used if that fragment encodes a modified substrate protein that interacts with a pathogen-specific protease and that retains its ability to interact with its corresponding NB-LRR protein. Alternatively, fragments of a nucleotide sequence that can be used as hybridization probes generally do not need to retain biological activity. Thus, fragments of the nucleic acid molecules can be at least 10, 15, 20, 25, 50, 75, 100, 125, 150, 175, 200, 250, 300, 350, 400, 450, 500, 550, 600, 650, 700, 750, 800, 850 or 900 nucleotides, or up to the number of nucleotides present in a full-length nucleic acid molecule.

A fragment of the nucleic acid molecule therefore can include a functionally/biologically active portion, or it can include a fragment that can be used as a hybridization probe or PCR primer. A biologically active portion of the nucleic acid molecule can be prepared by isolating part of the sequence of the nucleic acid molecule, operably linking that fragment to a promoter, expressing the nucleotide sequence encoding the protein, and assessing the amount or activity of the protein. Methods of assaying protein expression are well known in the art. See, e.g., Chan et al. (1994) J. Biol. Chem. 269:17635-17641; Freyssinet & Thomas (1998) Pure & Appl. Chem. 70:61-66; and Kirby et al. (2007) Adv. Clin. Chem. 44:247-292; as well as US Patent Application Publication Nos. 2009/0183286 and 2009/0217424; and U.S. Pat. Nos. 7,294,711 and 7,408,055. Likewise, kits for assaying protein expression are commercially available, for example, from Applied Biosystems, Inc. (Foster City, Calif.), Caliper Life Sciences (Hopkinton, Mass.), Promega (Madison, Wis.), and SABiosciences (Frederick, Md.). Protein expression also can be assayed using other methods well known in the art, including, but not limited to, Western blot analysis, enzyme-linked immunosorbent assay, and the like. See, e.g., Sambrook & Russel (2001), supra. Moreover, methods of assaying pathogen-specific protease substrate protein activity are well known in the art. See, DeYoung et al. (2012), supra.

For nucleotide sequences, “variant” means a substantially similar nucleotide sequence to a nucleotide sequence of a recombinant nucleic acid molecule as described herein, for example, a substantially similar nucleotide sequence encoding a modified substrate protein. For nucleotide sequences, a variant comprises a nucleotide sequence having deletions (i.e., truncations) at the 5′ and/or 3′ end, deletions and/or additions of one or more nucleotides at one or more internal sites compared to the nucleotide sequence of the recombinant nucleic acid molecules as described herein; and/or substitution of one or more nucleotides at one or more sites compared to the nucleotide sequence of the recombinant nucleic acid molecules described herein. One of skill in the art understands that variants are constructed in a manner to maintain the open reading frame.

Conservative variants include those nucleotide sequences that, because of the degeneracy of the genetic code (see, Table 1), result in a functionally active modified substrate protein as described herein. Naturally occurring allelic variants can be identified by using well-known molecular biology techniques such as, for example, polymerase chain reaction (PCR) and hybridization techniques. Variant nucleotide sequences also can include synthetically derived sequences, such as those generated, for example, by site-directed mutagenesis but which still provide a functionally active modified substrate protein. Generally, variants of a nucleotide sequence of the recombinant nucleic acid molecules as described herein will have at least 70%, 75%, 80%, 85%, 90%, 91%, 92%, 93%, 94%, 95%, 96%, 97%, 98%, 99% or more sequence identity to the nucleotide sequence of the recombinant nucleic acid molecules as determined by sequence alignment programs and parameters as described elsewhere herein.

When making recombinant nucleic acid molecules as described herein and variants thereof, one of skill in the art can be further guided by knowledge of redundancy in the genetic code as shown below in Table 1.

Deletions, insertions and/or substitutions of the nucleotide sequence of the recombinant nucleic acid molecules are not expected to produce radical changes in their characteristics. However, when it is difficult to predict the exact effect of the substitution, deletion or insertion in advance of doing so, one of skill in the art will appreciate that the effect can be evaluated by expression assays.

Variant nucleic acid molecules also encompass nucleotide sequences derived from a mutagenic and recombinogenic procedure such as DNA shuffling. With such a procedure, the nucleotide sequences of the recombinant nucleic acid molecules described herein can be manipulated to create a new nucleic acid molecule possessing the desired properties. In this manner, libraries of recombinant nucleic acid molecules can be generated from a population of related nucleic acid molecules comprising sequence regions that have substantial sequence identity and can be homologously recombined in vitro or in vivo. For example, using this approach, sequence motifs encoding a domain of interest can be shuffled between the nucleic acid molecules described herein and other known promoters to obtain a new nucleic acid molecule with an improved property such as increased promoter activity.

Methods of mutating and altering nucleotide sequences, as well as DNA shuffling, are well known in the art. See, Crameri et al. (1997) Nature Biotech. 15:436-438; Crameri et al. (1998) Nature 391:288-291; Kunkel (1985) Proc. Natl. Acad. Sci. USA 82:488-492; Kunkel et al. (1987) Methods in Enzymol. 154:367-382; Moore et al. (1997) J. Mol. Biol. 272:336-347; Stemmer (1994) Proc. Natl. Acad. Sci. USA 91:10747-10751; Stemmer (1994) Nature 370:389-391; Zhang et al. (1997) Proc. Natl. Acad. Sci. USA 94:4504-4509; and Techniques in Molecular Biology (Walker & Gaastra eds., MacMillan Publishing Co. 1983) and the references cited therein; as well as U.S. Pat. Nos. 4,873,192; 5,605,793 and 5,837,458. As such, the nucleic acid molecules as described herein can have many modifications.

Variants of the recombinant nucleic acid molecules described herein also can be evaluated by comparing the percent sequence identity between the polypeptide encoded by a variant and the polypeptide encoded by a reference nucleic acid molecule. Thus, for example, an isolated nucleic acid molecule can be one that encodes a polypeptide with a given percent sequence identity to the polypeptide of interest. Percent sequence identity between any two polypeptides can be calculated using sequence alignment programs and parameters described elsewhere herein. Where any given pair of polynucleotides of the present disclosure is evaluated by comparison of the percent sequence identity shared by the two polypeptides they encode, the percent sequence identity between the two encoded polypeptides can be at least 70%, 75%, 80%, 85%, 90%, 91%, 92%, 93%, 94%, 95%, 96%, 97%, 98%, 99% or more sequence identity.

Determining percent sequence identity between any two sequences can be accomplished using a mathematical algorithm. Non-limiting examples of such mathematical algorithms include, but are not limited to, the algorithm of Myers & Miller (1988) CABIOS 4:11-17; the local alignment algorithm of Smith et al. (1981) Adv. Appl. Math. 2:482-489; the global alignment algorithm of Needleman & Wunsch (1970) J. Mol. Biol. 48:443-453; the search-for-local alignment method of Pearson & Lipman (1988) Proc. Natl. Acad. Sci. USA 85:2444-2448; the algorithm of Karlin & Altschul (1990) Proc. Natl. Acad. Sci. USA 87:2264-2268, modified as in Karlin & Altschul (1993) Proc. Natl. Acad. Sci. USA 90:5873-5877.

The present disclosure therefore includes recombinant nucleic acid molecules having a nucleotide sequence that encodes a modified substrate protein of a pathogen-specific protease, where the modified substrate protein has a heterologous protease recognition sequence and can be incorporated into nucleic acid constructs such as expression cassettes and vectors.

### Nucleic Acid Constructs

Compositions of the present disclosure also include nucleic acid constructs, such as expression cassettes or vectors, having plant promoters operably linked with a nucleic acid molecule that encodes a substrate protein of a pathogen-specific protease and a heterologous pathogen-specific protease recognition sequence for use in transforming plant cells, plant parts and plants. In addition, the constructs can include a nucleic acid molecule that encodes a NB-LRR protein, particularly when such NB-LRR protein is not native/not endogenous to the plant cell, plant part or plant to be transformed.

As used herein, “nucleic acid construct” means an oligonucleotide or polynucleotide composed of deoxyribonucleotides, ribonucleotides or combinations thereof having incorporated therein the nucleotide sequences described herein. The nucleotide construct can be used for transforming organisms such as plants. In this manner, plant promoters operably linked to a nucleotide sequence for a modified substrate protein of a pathogen-specific protease as described herein are provided in nucleic acid constructs for expression in a plant cell, plant part or plant.

As used herein, “expression cassette” means a nucleic acid molecule having at least a control sequence operably linked to a coding sequence.

As used herein, “operably linked” means that the elements of the expression cassette are configured so as to perform their usual function. Thus, control sequences (i.e., promoters) operably linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence still can be considered “operably linked” to the coding sequence.

As used herein, a “coding sequence” or “coding sequences” means a sequence that encodes a particular polypeptide, and is a nucleotide sequence that is transcribed (in the case of DNA) and translated (in the case of mRNA) into a polypeptide in vitro or in vivo when placed under the control of appropriate regulatory sequences. The boundaries of the coding sequence are determined by a start codon at a 5′ (amino) terminus and a translation stop codon at a 3′ (carboxy) terminus. A coding sequence can include, but is not limited to, viral nucleic acid sequences, cDNA from prokaryotic or eukaryotic mRNA, genomic DNA sequences from prokaryotic or eukaryotic DNA, and even synthetic DNA sequences. A transcription termination sequence will usually be located 3′ to the coding sequence. Examples of coding sequences for use herein include nucleotide sequence that encodes a modified substrate protein of a pathogen-specific protease, a NB-LRR protein or both.

As used herein, “control sequence” or “control sequences” means promoters, polyadenylation signals, transcription and translation termination sequences, upstream regulatory domains, origins of replication, internal ribosome entry sites (“IRES”), enhancers, and the like, which collectively provide for replication, transcription and translation of a coding sequence in a recipient host cell. Not all of these control sequences need always be present so long as the selected coding sequence is capable of being replicated, transcribed and translated in an appropriate host cell.

As used herein, a “promoter” means a nucleotide region comprising a nucleic acid (i.e., DNA) regulatory sequence, wherein the regulatory sequence is derived from a gene or synthetically created that is capable of binding RNA polymerase and initiating transcription of a downstream (3′-direction) coding sequence. A number of promoters can be used in the expression cassette, including the native promoter of the modified substrate protein or NB-LRR protein.

Alternatively, promoters can be selected based upon a desired outcome. Such promoters include, but are not limited to, “constitutive promoters” (where expression of a polynucleotide sequence operably linked to the promoter is unregulated and therefore continuous), “inducible promoters” (where expression of a polynucleotide sequence operably linked to the promoter is induced by an analyte, cofactor, regulatory protein, etc.), and “repressible promoters” (where expression of a polynucleotide sequence operably linked to the promoter is repressed by an analyte, cofactor, regulatory protein, etc.).

As used herein, “plant promoter” means a promoter that drives expression in a plant such as a constitutive, inducible (e.g., chemical-, environmental-, pathogen- or wound-inducible), repressible, tissue-preferred or other promoter for use in plants.

Examples of constitutive promoters include, but are not limited to, the rice actin 1 promoter (Wang et al. (1992) Mol. Cell. Biol. 12:3399-3406; and U.S. Pat. No. 5,641,876), the CaMV 19S promoter (Lawton et al. (1987) Plant Mol. Biol. 9:315-324), the CaMV 35S promoter (Odell et al. (1985) Nature 313:810-812), the nos promoter (Ebert et al. (1987) Proc. Natl. Acad. Sci. USA 84:5754-5749), the Adh promoter (Walker et al. (1987) Proc. Natl. Acad. Sci. USA 84:6624-6628), the sucrose synthase promoter (Yang & Russell (1990) Proc. Natl. Acad. Sci. USA 87:4144-4148), the ubiquitin promoters, and the like. See also, U.S. Pat. Nos. 5,608,149; 5,608,144; 5,604,121; 5,569,597; 5,466,785; 5,399,680; 5,268,463; 5,608,142 and 6,177,611.

Examples of chemical-inducible promoters include, but are not limited to, the maize Tn2-2 promoter, which is activated by benzenesulfonamide herbicide safeners; the maize GST promoter, which is activated by hydrophobic electrophilic compounds that are used as pre-emergent herbicides; and the tobacco PR-1a promoter, which is activated by salicylic acid. Other chemical-inducible promoters of interest include steroid-responsive promoters (e.g., the glucocorticoid-inducible promoters in Aoyama & Chua (1997) Plant J. 11:605-612; McNellis et al. (1998) Plant J. 14:247-257; and Schena et al. (1991) Proc. Natl. Acad. Sci. USA 88:10421-10425); tetracycline-inducible and tetracycline-repressible promoters (Gatz et al. (1991) Mol. Gen. Genet. 227:229-237; as well as U.S. Pat. Nos. 5,814,618 and 5,789,156); ABA- and turgor-inducible promoters, the auxin-binding protein gene promoter (Schwob et al. (1993) Plant J. 4:423-432), the UDP glucose flavonoid glycosyl-transferase gene promoter (Ralston et al. (1988) Genetics 119:185-187), the MPI proteinase inhibitor promoter (Cordero et al. (1994) Plant J. 6:141-150), and the glyceraldehyde-3-phosphate dehydrogenase gene promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Chemical-inducible promoters therefore can be used to modulate the expression of a nucleotide sequence of interest in a plant by applying an exogenous chemical regulator. Depending upon the objective, the promoter can be a chemical-inducible promoter, whereby application of the chemical induces gene expression, or a chemical-repressible promoter, whereby application of the chemical represses gene expression. See also, Gatz (1997) Annu. Rev. Plant Physiol. Plant Mol. Biol. 48:89.

Other inducible promoters include promoters from genes inducibly regulated in response to environmental stress or stimuli such as drought, pathogens, salinity and wounds. See, Graham et al. (1985) J. Biol. Chem. 260:6555-6560; Graham et al. (1985) J. Biol. Chem. 260:6561-6564; and Smith et al. (1986) Planta 168:94-100. Wound-inducible promoters include the metallocarboxypeptidase-inhibitor protein promoter (Graham et al. (1981) Biochem. Biophys. Res. Comm. 101:1164-1170).

Examples of tissue-preferred promoters include, but are not limited to, the rbcS promoter, the ocs, nos and mas promoters that have higher activity in roots or wounded leaf tissue, a truncated (−90 to +8) 35S promoter that directs enhanced expression in roots, an α-tubulin gene promoter that directs expression in roots, as well as promoters derived from zein storage protein genes that direct expression in endosperm. Additional examples of tissue-preferred promoters include, but are not limited to, the promoters of genes encoding the seed storage proteins (e.g., β-conglycinin, cruciferin, napin and phaseolin), zein or oil body proteins (e.g., oleosin), or promoters of genes involved in fatty acid biosynthesis (e.g., acyl carrier protein, stearoyl-ACP desaturase and fatty acid desaturases (e.g., fad 2-1)), and promoters of other genes expressed during embryo development (e.g., Bce4; Kridl et al. (1991) Seed Sci. Res. 1:209-219). Further examples of tissue-specific promoters include, but are not limited to, the lectin promoter (Lindstrom et al. (1990) Dev. Genet. 11:160-167; and Vodkin (1983) Prog. Clin. Biol. Res. 138:87-98), the corn alcohol dehydrogenase 1 promoter (Dennis et al. (1984) Nucleic Acids Res. 12:3983-4000; and Vogel et al. (1989) J. Cell. Biochem. 13:Part D, M350 (Abstract)), corn light harvesting complex (Bansal et al. (1992) Proc. Natl. Acad. Sci. USA 89:3654-3658; and Simpson (1986) Science 233:34-380), corn heat shock protein (Odell et al. (1985) Nature 313:810-812; and Rochester et al. (1986) EMBO J. 5:451-458), the pea small subunit RuBP carboxylase promoter (Cashmore, “Nuclear genes encoding the small subunit of ribulose-1,5-bisphosphate carboxylase” 29-38 In: Gen. Eng. of Plants (Plenum Press 1983); and Poulsen et al. (1986) Mol. Gen. Genet. 205:193-200), the Ti plasmid mannopine synthase promoter (Langridge et al. (1989) Proc. Natl. Acad. Sci. USA 86:3219-3223), the Ti plasmid nopaline synthase promoter (Langridge et al. (1989), supra), the Petunia chalcone isomerase promoter (van Tunen et al. (1988) EMBO J. 7:1257-1263), the bean glycine rich protein 1 promoter (Keller et al. (1989) Genes Dev. 3:1639-1646), the truncated CaMV 35s promoter (Odell et al. (1985), supra), the potato patatin promoter (Wenzler et al. (1989) Plant Mol. Biol. 13:347-354), the root cell promoter (Yamamoto et al. (1990) Nucleic Acids Res. 18:7449), the maize zein promoter (Langridge et al. (1983) Cell 34:1015-1022; Kriz et al. (1987) Mol. Gen. Genet. 207:90-98; Reina et al. (1990) Nucleic Acids Res. 18:6425; Reina et al. (1990) Nucleic Acids Res. 18:7449; and Wandelt et al. (1989) Nucleic Acids Res. 17:2354), the globulin-1 gene (Belanger et al. (1991) Genetics 129:863-872), the α-tubulin, cab promoter (Sullivan et al. (1989) Mol. Gen. Genet. 215:431-440), the PEPCase promoter (Hudspeth & Grula (1989) Plant Mol. Biol. 12:579-589), the R gene complex-associated promoters (Chandler et al. (1989) Plant Cell 1:1175-1183), and the chalcone synthase promoters (Franken et al. (1991) EMBO J. 10:2605-2612). See also, Canevascini et al. (1996) Plant Physiol. 112:513-524; Guevara-Garcia et al. (1993) Plant J. 4:495-505; Hansen et al. (1997) Mol. Gen. Genet. 254:337-343; Kawamata et al. (1997) Plant Cell Physiol. 38:792-803; Lam (1994) Results Probl. Cell Differ. 20:181-196; Matsuoka et al. (1993) Proc. Natl. Acad. Sci. USA 90:9586-9590; Orozco et al. (1993) Plant Mol. Biol. 23:1129-1138; Rinehart et al. (1996) Plant Physiol. 112:1331-1341; Russell et al. (1997) Transgenic Res. 6:157-168; Van Camp et al. (1996) Plant Physiol. 112:525-535; Yamamoto et al. (1994) Plant Cell Physiol. 35:773-778; and Yamamoto et al. (1997) Plant J. 12:255-265.

In some instances, the tissue-preferred promoter can be a leaf-preferred promoter. See, Gan et al. (1995) Science 270:1986-1988; Gotor et al. (1993) Plant J. 3:509-518; Kwon et al. (1994) Plant Physiol. 105:357-367; Matsuoka et al. (1993), supra; Orozco et al. (1993), supra; Yamamoto et al. (1994), supra; and Yamamoto et al. (1997), supra.

In some instances, the tissue-preferred promoter can be a root-preferred promoter. See, Capana et al. (1994) Plant Mol. Biol. 25:681-691 (rolB promoter); Hire et al. (1992) Plant Mol. Biol. 20:207-218 (soybean root-specific glutamine synthetase gene); Keller & Baumgartner (1991) Plant Cell 3:1051-1061 (root-specific control element in the GRP 1.8 gene of French bean); Kuster et al. (1995) Plant Mol. Biol. 29:759-772 (VfENOD-GRP3 gene promoter) Miao et al. (1991) Plant Cell 3:11-22 (full-length cDNA clone encoding cytosolic glutamine synthetase (GS), which is expressed in roots and root nodules of soybean); and Sanger et al. (1990) Plant Mol. Biol. 14:433-443 (root-specific promoter of the mannopine synthase (MAS) gene of A. tumefaciens); see also, U.S. Pat. Nos. 5,837,876; 5,750,386; 5,633,363; 5,459,252; 5,401,836; 5,110,732; and 5,023,179. Likewise, Bogusz et al. (1990) Plant Cell 2:633-641 describes two root-specific promoters isolated from hemoglobin genes from the nitrogen-fixing nonlegume Parasponia andersonii and the related non-nitrogen-fixing nonlegume Trema tomentosa. Leach & Aoyagi (1991) Plant Sci. 79:69-76 describes an analysis of the promoters of the highly expressed rolC and rolD root-inducing genes of Agrobacterium rhizogenes. Teeri et al. (1989) EMBO J. 8:343-335 describes a gene fusion to lacZ to show that the Agrobacterium T-DNA gene encoding octopine synthase is especially active in the epidermis of the root tip and that the TR2′ gene is root specific in the intact plant and stimulated by wounding in leaf tissue.

In some instances, the tissue-preferred promoter can be a seed-preferred promoter, which includes both “seed-specific” promoters (i.e., promoters active during seed development such as promoters of seed storage proteins) and “seed-germinating” promoters (i.e., promoters active during seed germination). See, Thompson et al. (1989) BioEssays 10:108-113. Examples of seed-preferred promoters include, but are not limited to, the Cim1 promoter (cytokinin-induced message); the cZ19B1 promoter (maize 19 kDa zein); the myo-inositol-1-phosphate synthase (milps) promoter (Int'l Patent Application Publication No. WO 00/11177; and U.S. Pat. No. 6,225,529); the γ-zein promoter; and the globulin 1 (Glb-1) promoter. For monocots, seed-specific promoters include, but are not limited to, promoters from maize 15 kDa zein, 22 kDa zein, 27 kDa zein, γ-zein, waxy, shrunken 1, shrunken 2 and Glb-1. See also, Int'l Patent Application Publication No. WO 00/12733, which discloses seed-preferred promoters from end1 and end2 genes. For dicots, seed-specific promoters include, but are not limited to, promoters from bean β-phaseolin, napin, β-conglycinin, soybean lectin, cruciferin and pea vicilin (Czako et al. (1992) Mol. Gen. Genet. 235:33-40). See also, U.S. Pat. No. 5,625,136.

In some instances, the tissue-preferred promoter can be a stalk-preferred promoter. Examples of stalk-preferred promoters include, but are not limited to, the maize MS8-15 gene promoter (Int'l Patent Application Publication No. WO 98/00533; and U.S. Pat. No. 5,986,174), and the promoters disclosed in Graham et al. (1997) Plant Mol. Biol. 33:729-735.

In some instances, the tissue-preferred promoter can be a vascular tissue-preferred promoter. For example, a vascular tissue-preferred promoter can be used to express the modified substrate protein in polypexylem and phloem tissue. Examples of vascular tissue-preferred promoters include, but are not limited to, the Prunus serotina prunasin hydrolase gene promoter (Int'l Patent Application Publication No. WO 03/006651), and the promoters disclosed in U.S. Pat. No. 6,921,815.

As an alternative to the promoters listed above, in some instances a low level of expression is desired and can be achieved by using a weak promoter. As used herein, “weak promoter” means a promoter that drives expression of a coding sequence at a low level. As used herein, “low level” means at levels of about 1/1000 transcripts to about 1/100,000 transcripts to about 1/500,000 transcripts. Alternatively, it is recognized that weak promoter also encompasses promoters that are expressed in only a few cells and not in others to give a total low level of expression. Where a promoter is expressed at unacceptably high levels, portions of the promoter sequence can be deleted or modified to decrease expression levels.

Examples of weak constitutive promoters include, but are not limited to, the core promoter of the Rsyn7 promoter (Int'l Patent Application Publication No. WO 99/43838 and U.S. Pat. No. 6,072,050), the core 35S CaMV promoter, and the like. Other exemplary weak constitutive promoters are described, for example, in U.S. Pat. Nos. 5,608,149; 5,608,144; 5,604,121; 5,569,597; 5,466,785; 5,399,680; 5,268,463; 5,608,142 and 6,177,611.

Weak promoters can be used when designing expression cassettes for NB-LRR proteins, as NB-LRR genes preferably are constitutively expressed at low levels because high levels can lead to cell death in the absence of pathogens.

The expression cassette can include other control sequences 5′ to the coding sequence. For example, the expression cassette can include a 5′ leader sequence, which can act to enhance translation. Examples of 5′ leader sequences include, but are not limited to, picornavirus leaders (e.g., encephalomyocarditis virus (EMCV) leader; Elroy-Stein et al. (1989) Proc. Natl. Acad. Sci. USA 86:6126-6130); potyvirus leaders (e.g., tobacco etch virus (TEV) leader; Gallie et al. (1995) Gene 165:233-238); maize dwarf mosaic virus (MDMV) leader (Allison et al. (1986) Virology 154:9-20); human immunoglobulin heavy-chain binding protein (BiP; Macejak et al. (1991) Nature 353:90-94); untranslated leader from the coat protein mRNA of alfalfa mosaic virus (AMV RNA 94; Jobling et al. (1987) Nature 325:622-625); tobacco mosaic virus (TMV) leader (Gallie et al., “Eukaryotic viral 5′-leader sequences act as translational enhancers in eukaryotes and prokaryotes” 237-256 In: Molecular Biology of RNA (Cech ed., Liss 1989)); and maize chlorotic mottle virus (MCMV) leader (Lommel et al. (1991) Virology 81:382-385). See also, Della-Cioppa et al. (1987) Plant Physiol. 84:965-968; and Gallie (1996) Plant Mol. Biol. 32:145-158. Other methods or sequences known to enhance translation also can be used, for example, introns, and the like.

The expression cassette also can include a coding sequence for the modified substrate protein of the pathogen-specific protease and/or NB-LRR protein. As discussed above, the modified substrate protein includes a heterologous protease recognition sequence. The heterologous protease recognition sequence can be located within, for example, an exposed loop of the substrate protein. As noted above, nucleic and amino acid sequences are well known in the art for many protease recognition sequences that can be inserted into the substrate protein such as PBS1. In addition, nucleic and amino acid sequences are known in the art for various NB-LRR proteins. These sequences can be used when constructing the expression cassette(s).

For example, the coding sequence can be SEQ ID NO:9 (modified PBS1 having an AvrRpt2 protease recognition sequence) operably linked to the native PBS1 promoter. Likewise, the coding sequence can include a NB-LRR protein such as RPS5 when the modified substrate protein is based upon PBS1.

The control sequence(s) and/or the coding sequence therefore can be native/analogous to the host cell or to each other. Alternatively, the control sequence(s) and/or coding sequence can be heterologous to the host cell or to each other. As used herein, “heterologous” means a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The expression cassette also can include a transcriptional and/or translational termination region that is functional in plants. The termination region can be native with the transcriptional initiation region (i.e., promoter), can be native with the operably linked coding sequence, can be native with the plant of interest, or can be derived from another source (i.e., foreign or heterologous to the promoter, the coding sequence, the plant host cell, or any combination thereof). Termination regions are typically located downstream (3′-direction) from the coding sequence. Termination regions include, but are not limited to, the potato proteinase inhibitor (PinII) gene or the Ti-plasmid of A. tumefaciens, such as the octopine synthase and nopaline synthase termination regions. See e.g., Ballas et al. (1989) Nucleic Acids Res. 17:7891-7903; Guerineau et al. (1991) Mol. Gen. Genet. 262:141-144; Joshi et al. (1987) Nucleic Acid Res. 15:9627-9639; Mogen et al. (1990) Plant Cell 2:1261-1272; Munroe et al. (1990) Gene 91:151-158; Proudfoot (1991) Cell 64:671-674; and Sanfacon et al. (1991) Genes Dev. 5:141-149.

The expression cassette also can include one or more linkers. As used herein, “linker” means a nucleotide sequence that functions to link one element of the expression cassette with another without otherwise contributing to the transcription or translation of a nucleotide sequence of interest when present in the expression cassette. The linker can include plasmid sequences, restriction sequences and/or sequences of a 5′-untranslated region (5′-UTR). Alternatively, the linker further can include nucleotide sequences encoding the additional amino acid residues that naturally flank the heterologous protease recognition sequence in the substrate protein from which it was isolated. The length and sequence of the linker can vary and can be about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 600, 700, 800, 900, 1000 nucleotides or greater in length.

Just as expression of the modified substrate protein and/or NB-LRR protein can be targeted to specific tissues or cell types by appropriate use of promoters, it also can be targeted to different locations within a cell of a plant host by appropriate use of signal and/or targeting peptide sequences. Unlike a promoter, which acts at the transcriptional level, signal and/or targeting peptide sequences are part of the initial translation product. Therefore, the expression cassette also can include a signal and/or targeting peptide sequence. Examples of such sequences include, but are not limited to, the transit peptide for the acyl carrier protein, the small subunit of RUBISCO, plant EPSP synthase, and the like. See, Archer et al. (1990) J. Bioenerg. Biomemb. 22:789-810; Clark et al. (1989) J. Biol. Chem. 264:17544-17550; Daniell (1999) Nat. Biotech. 17:855-856; de Castro Silva Filho et al. (1996) Plant Mol. Biol. 30:769-780; Della-Cioppa et al. (1987) Plant Physiol. 84:965-968; Lamppa et al. (1988) J. Biol. Chem. 263:14996-14999; Lawrence et al. (1997) J. Biol. Chem. 272:20357-20363; Romer et al. (1993) Biochem. Biophys. Res. Commun. 196:1414-1421; Schmidt et al. (1993) J. Biol. Chem. 268:27447-27457; Schnell et al. (1991) J. Biol. Chem. 266:3335-3342; Shah et al. (1986) Science 233:478-481; Von Heijne et al. (1991) Plant Mol. Biol. Rep. 9:104-126; and Zhao et al. (1995) J. Biol. Chem. 270:6081-6087; as well as U.S. Pat. No. 6,338,168.

It may be desirable to locate the modified substrate protein and/or NB-LRR protein on specific plant membranes such as the plasma membrane or tonoplast membrane. This can be accomplished, for example, by adding specific amino acid sequences to the N-terminus of these proteins by adding specific sequences to the expression cassette as described in Raikhel & Chrispeels, “Protein sorting and vesicle traffic” In: Biochemistry and Molecular Biology of Plants (Buchanan et al. eds., American Society of Plant Physiologists 2000). See also, Denecke et al. (1992) EMBO J. 11:2345-2355; Denecke et al. (1993) J. Exp. Bot. 44:213-221; Gomord et al. (1996) Plant Physiol. Biochem. 34:165-181; Lehmann et al. (2001) Plant Physiol. 127:436-449; Munro & Pelham (1986) Cell 46:291-300; Munro & Pelham (1987) Cell 48:899-907; Vitale et al. (1993) J. Exp. Bot. 44:1417-1444; and Wandelt et al. (1992) Plant J. 2:181-192.

Additional guidance on subcellular targeting of proteins in plants can be found, for example, in Bruce (2001) Biochim Biophys Acta 1541:2-21; Emanuelsson et al. (2000) J. Mol. Biol. 300:1005-1016; Emanuelsson & von Heijne (2001) Biochim Biophys Acta 1541:114-119; Hadlington & Denecke (2000) Curr. Opin. Plant Biol. 3:461-468; Nicchitta (2002) Curr. Opin. Cell Biol. 14:412-416; and Silva-Filho (2003) Curr. Opin. Plant Biol. 6:589-595.

The expression cassette also can include nucleotide sequences encoding agronomic and pesticidal polypeptides, and the like. Such sequences can be stacked with any combination of nucleotide sequences to create plant cells, plants parts and plants with a desired phenotype. For example, the nucleic acid molecule encoding modified substrate protein and/or NB-LRR protein can be stacked with nucleotide sequences encoding a pesticidal polypeptide such as a δ-endotoxin. The combinations generated also can include multiple copies of any one of the nucleotide sequences of interest. Examples of other nucleotide sequences of interest include, but are not limited to, sequences encoding for high oil (U.S. Pat. No. 6,232,529); balanced amino acids (hordothionins; U.S. Pat. Nos. 5,703,409; 5,885,801; 5,885,802 and 5,990,389); barley high lysine (Williamson et al. (1987) Eur. J. Biochem. 165:99-106; and Int'l Patent Application Publication No. WO 98/20122); high methionine proteins (Pedersen et al. (1986) J. Biol. Chem. 261:6279-6284; Kirihara et al. (1988) Gene 71:359-370; and Musumura et al. (1989) Plant Mol. Biol. 12:123-130); increased digestibility (modified storage proteins; U.S. Pat. No. 6,858,778); and thioredoxins (U.S. Pat. No. 7,009,087).

The nucleotide sequence encoding the modified substrate protein and/or NB-LRR disease resistance protein also can be stacked with nucleotide sequences encoding polypeptides for herbicide resistance (e.g., glyphosate or HPPD resistance; see, e.g., EPSPS genes, GAT genes (Int'l Patent Application Publication Nos. WO 02/36782 and WO 03/092360; and US Patent Application Publication No. 2004/0082770); lectins (Van Damme et al. (1994) Plant Mol. Biol. 24:825-830); fumonisin detoxification (U.S. Pat. No. 5,792,931); acetolactate synthase (ALS) mutants that lead to herbicide resistance such as the S4 and/or Hra mutations; inhibitors of glutamine synthase such as phosphinothricin or basta (e.g., bar gene); modified starches (ADPG pyrophosphorylases (AGPase), starch synthases (SS), starch branching enzymes (SBE) and starch debranching enzymes (SDBE)); and polymers or bioplastics (U.S. Pat. No. 5,602,321); beta-ketothiolase, polyhydroxybutyrate synthase and acetoacetyl-CoA reductase (Schubert et al. (1988) J. Bacteriol. 170:5837-5847).

The nucleotide sequence encoding the modified substrate protein and/or NB-LRR disease resistance protein also can be stacked with nucleotide sequences encoding for agronomic traits such as male sterility (U.S. Pat. No. 5,583,210), stalk strength, flowering time or transformation technology traits such as cell cycle regulation or gene targeting (Int'l Patent Application Publication Nos. and WO 99/25821; WO 99/61619 and WO 00/17364).

These stacked combinations can be created by any method including, but not limited, to cross breeding plants by any conventional or TOPCROSS™ methodology (DuPont Specialty Grains; Des Moines, Iowa), zinc finger nucleases (ZFNs), transcription activator-like effector nucleases (TALENs) or other genetic transformation. If the traits are stacked by genetically transforming the plants, the nucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate expression cassettes (trans) or contained on the same transformation cassette (cis). Expression of the sequences can be driven by the same promoter or by different promoters. In certain instances, it may be desirable to introduce an expression cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, Int'l Patent Application Publication Nos. WO 99/25821; WO 99/25840; WO 99/25853; WO 99/25854 and WO 99/25855.

In addition to the above, it is contemplated that the nucleic acid constructs can be used in the form of a system, particularly when used in plant cells, plant parts and plants that lack a substrate protein of a pathogen-specific protease and NB-LRR protein pair. Such systems can include one or more nucleic acid constructs, such as expression cassettes or vectors, having a promoter that drives expression in a plant, plant part or plant cell operably linked to a coding sequence for a modified substrate protein of a pathogen-specific protease, where the substrate protein has a heterologous protease recognition sequence, and a sequence for a promoter that drives expression in a plant, plant part or plant cell operably linked to a coding sequence for a NB-LRR protein. The promoters can be the same or can be distinct. For example, the first promoter can be an inducible promoter and the second promoter can be a constitutive promoter, especially a weak constitutive promoter. Alternatively, both the first and second promoters can be inducible, repressible or constitutive. The NB-LRR protein can associate with, and can be activated by, the modified substrate. Such systems therefore can be used to provide the protein pair to a plant cell, plant part or plant that does not natively express the protein pair.

Alternatively, the system can include a first nucleic acid construct having nucleotide sequence for a promoter that drives expression in a plant cell, plant part or plant operably linked to a coding sequence for a modified substrate protein of a pathogen-specific protease as described herein, and a second nucleic acid construct having a nucleotide sequence for a promoter that drives expression in a plant cell, plant part or plant operably linked to a coding sequence for a NB-LRR protein.

Additional nucleic acid constructs also can be included in the system, where each construct has a nucleotide sequence that encodes a distinct modified substrate protein, each having a heterologous recognition sequence for a separate pathogen-specific protease. Although each modified substrate protein has a heterologous recognition sequence distinct from one another, each can associate with, and can activate, the NB-LRR protein.

Regardless of whether used as individual nucleic acid constructs or systems, and where appropriate, the nucleotide sequences can be optimized for increased expression in plants. That is, the nucleotide sequences can be synthesized using plant-preferred codons for improved expression. Methods for optimizing nucleotide sequences for expression in plants are well known in the art. See, Campbell & Gowri (1990) Plant Physiol. 92:1-11; Murray et al. (1989) Nucleic Acids Res. 17:477-498; and Wada et al. (1990) Nucl. Acids Res. 18:2367-2411; as well as U.S. Pat. Nos. 5,096,825; 5,380,831; 5,436,391; 5,625,136; 5,670,356 and 5,874,304.

Likewise, additional sequence modifications are known to enhance nucleotide sequence expression in plants. These include elimination of sequences encoding spurious polyadenylation signals, exon-intron splice site signals, transposon-like repeats, and other such well-characterized sequences that may be deleterious to gene expression. The G-C content of the sequence can be adjusted to levels average for a given cellular host, as calculated by reference to known genes expressed in the host plant. When possible, the nucleotide sequence can be modified to avoid predicted hairpin secondary mRNA structures.

Suitable methods of constructing expression cassettes are well known in the art and can be found, for example, in Balbás & Lorence, Recombinant Gene Expression: Reviews and Protocols, 2nd ed. (Humana Press 2004); Davis et al., Basic Methods in Molecular Biology (Elsevier Press 1986); Sambrook & Russell (2001), supra; Tijssen, Laboratory Techniques in Biochemistry and Molecular Biology—Hybridization with Nucleic Acid Probes (Elsevier 1993); Ausubel et al. (1995), supra; as well as U.S. Pat. Nos. 6,664,387; 7,060,491; 7,345,216 and 7,494,805.

The expression cassette therefore can include at least, in the direction of transcription (i.e., 5′ to 3′ direction), a plant promoter that is functional in a plant cell, plant part or plant operably linked to a nucleotide sequence encoding a modified substrate protein having a heterologous protease recognition sequence. In some instances, the expression cassette also can include a nucleotide sequence encoding a NB-LRR disease resistance protein.

To assist in introducing the nucleotide sequences of interest into the appropriate host cells, the expression cassette can be incorporated or ligated into a vector. As used herein, “vector” means a replicon, such as a plasmid, phage or cosmid, to which another nucleic acid segment may be attached so as to bring about the replication of the attached segment. A vector is capable of transferring nucleic acid molecules to the host cells. Bacterial vectors typically can be of plasmid or phage origin.

Typically, the terms “vector construct,” “expression vector,” “gene expression vector,” “gene delivery vector,” “gene transfer vector,” and “expression cassette” all refer to an assembly that is capable of directing the expression of a sequence or gene of interest. Thus, the terms include cloning and expression vehicles.

Vectors typically contain one or a small number of restriction endonuclease recognition sites where a nucleic acid molecule of interest can be inserted in a determinable fashion without loss of essential biological function of the vector, as well as a selectable marker that can be used for identifying and selecting cells transformed with the vector.

A vector therefore can be capable of transferring nucleic acid molecule to target cells (e.g., bacterial plasmid vectors, particulate carriers and liposomes). The selection of vector will depend upon the preferred transformation technique and the target species for transformation. The most commonly used plant transformation vectors are binary vectors because of their ability to replicate in intermediate host cells such as E. coli and A. tumefaciens. The intermediate host cells allow one to increase the copy number of the cloning vector and/or to mediate transformation of a different host cell. With an increased copy number, the vector containing the expression cassette of interest can be isolated in significant quantities for introduction into the desired plant. General descriptions of plant vectors can be found, for example, in Gruber et al., “Vectors for plant transformation” 89-119 In: Methods in Plant Molecular Biology & Biotechnology (Glich et al. eds., CRC Press 1993). Examples of vectors for use with A. tumefaciens can be found, for example, in U.S. Pat. No. 7,102,057.

Restriction enzymes can be used to introduce cuts into the target nucleic acid molecule (e.g., nucleotide sequence encoding a modified substrate protein and/or NB-LRR protein) and the plasmid to facilitate insertion of the target into the vector such as a plasmid. Moreover, restriction enzyme adapters such as EcoRI/NotI adapters can be added to the target mRNA when the desired restriction enzyme sites are not present within it. Methods of adding restriction enzyme adapters are well known in the art. See, Krebs et al. (2006) Anal. Biochem. 350:313-315; and Lonneborg et al. (1995), supra. Likewise, kits for adding restriction enzyme sites are commercially available, for example, from Invitrogen (Carlsbad, Calif.).

Alternatively, viruses such as bacteriophages can be used as the vector to deliver the target mRNA to competent host cells. Vectors can be constructed using standard molecular biology techniques as described, for example, in Sambrook & Russell (2001), supra.

As noted above, selectable markers can be used to identify and select transformed plants, plant parts or plant host cells. Selectable markers include, but are not limited to, nucleotide sequences encoding antibiotic resistance, such as those encoding neomycin phosphotransferase II (NEO), hygromycin phosphotransferase (HPT), as well as nucleotide sequences encoding resistance to ampicillin, kanamycin, spectinomycin or tetracycline, and even nucleotide sequences encoding herbicidal compounds such as glufosinate ammonium, bromoxynil, imidazolinones and 2,4-dichlorophenoxyacetate (2,4-D).

Additional selectable markers can include phenotypic markers such as nucleic acid sequences encoding β-galactosidase, β-glucoronidase (GUS; Jefferson (1987) Plant Mol. Biol. Rep. 5:387-405); luciferase (Teeri et al. (1989) EMBO J. 8:343-350); anthocyanin production (Ludwig et al. (1990) Science 247:449-450), and fluorescent proteins such as green fluorescent protein (GFP; Chalfie et al. (1994) Science 263:802-805; Fetter et al. (2004) Plant Cell 16:215-228; and Su et al. (2004) Biotechnol. Bioeng. 85:610-619); cyan fluorescent protein (CYP; Bolte et al. (2004) J. Cell Science 117:943-954; and Kato et al. (2002) Plant Physiol. 129:913-942), and yellow fluorescent protein (PhiYFP™, available from Evrogen (Moscow, Russia); Bolte et al. (2004) J. Cell Science 117:943-954). For additional selectable markers, Baim et al. (1991) Proc. Natl. Acad. Sci. USA 88:5072-5076; Barkley & Bourgeois, “Repressor recognition of operator and effectors” 177-120 In: The Operon (Miller & Reznikoff eds., Cold Spring Harbor Laboratory Press 1980); Bonin (1993) Ph.D. Thesis, University of Heidelberg; Brown et al. (1987) Cell 49:603-612; Christopherson et al. (1992) Proc. Natl. Acad. Sci. USA 89:6314-6318; Degenkolb et al. (1991) Antimicrob. Agents Chemother. 35:1591-1595; Deuschle et al. (1989) Proc. Natl. Acad. Sci. USA 86:5400-5404; Deuschle et al. (1990) Science 248:480-483; Figge et al. (1988) Cell 52:713-722; Fuerst et al. (1989) Proc. Natl. Acad. Sci. USA 86:2549-2553; Gill et al. (1988) Nature 334:721-724; Gossen et al. (1992) Proc. Natl. Acad. Sci. USA 89:5547-5551; Gossen (1993) Ph.D. Thesis, University of Heidelberg; Hillenand-Wissman (1989) Topics Mol. Struc. Biol. 10:143-162; Hlavka et al., Handbook of Experimental Pharmacology, Vol. 78 (Springer-Verlag 1985); Hu et al. (1987) Cell 48:555-566; Kleinschnidt et al. (1988) Biochemistry 27:1094-1104; Labow et al. (1990) Mol. Cell. Biol. 10:3343-3356; Oliva et al. (1992) Antimicrob. Agents Chemother. 36:913-919; Reines et al. (1993) Proc. Natl. Acad. Sci. USA 90:1917-1921; Reznikoff (1992) Mol. Microbiol. 6:2419-2422; Yao et al. (1992) Cell 71:63-72; Yarranton (1992) Curr. Opin. Biotech. 3:506-511; Wyborski et al. (1991) Nucleic Acids Res. 19:4647-4653; and Zambretti et al. (1992) Proc. Natl. Acad. Sci. USA 89:3952-3956. The above list of selectable markers is not intended to be limiting, as any selectable marker can be used.

The vector therefore can be selected to allow introduction of the expression cassette into the appropriate host cell such as a plant host cell. Bacterial vectors are typically of plasmid or phage origin. Appropriate bacterial cells are infected with phage vector particles or transfected with naked phage vector DNA. If a plasmid vector is used, the cells are transfected with the plasmid vector DNA.

The present disclosure therefore includes nucleotide constructs such as expression cassettes and vectors having a nucleotide sequence encoding a modified substrate protein of a pathogen-specific protease and a heterologous protease recognition sequence. In addition, the nucleic acid constructs can include a nucleotide sequence encoding a NB-LRR protein. The nucleic acid constructs can be introduced into an organism such as a plant to confer resistance to plant pathogens expressing specific proteases.

### Recombinant Peptides, Polypeptides and Proteins

Compositions of the present disclosure also include isolated or purified, modified substrate proteins of a pathogen-specific protease, where the substrate proteins have heterologous protease recognition sequences, as well as fragments and/or variants thereof. Methods for producing peptide, polypeptides and proteins in plant cells, plant parts and plants are discussed elsewhere herein.

Methods of isolating or purifying peptides, polypeptides and proteins are well known in the art. See, Ehle & Horn (1990) Bioseparation 1:97-110; Hengen (1995) Trends Biochem Sci. 20:285-286; Basic Methods in Protein Purification and Analysis: A Laboratory Manual (Simpson et al. eds., Cold Spring Harbor Laboratory Press 2008); Regnier (1983) Science 222:245-252; Shaw, “Peptide purification by reverse-phase HPLC” 257-287 In: Methods in Molecular Biology, Vol. 32 (Walker ed., Humana Press 1994); as well as US Patent Application Publication No. 2009/0239262; and U.S. Pat. Nos. 5,612,454; 7,083,948; 7,122,641; 7,220,356 and 7,476,722.

As used herein, “peptide,” “polypeptide” and “protein” are used interchangeably to mean a polymer of amino acid residues. The terms apply to amino acid polymers in which one or more amino acid residues is an artificial chemical analogue of a corresponding naturally occurring amino acid, as well as to naturally occurring amino acid polymers.

As used herein, “residue,” “amino acid residue” and “amino acid” are used interchangeably to mean an amino acid that is incorporated into a molecule such as a peptide, polypeptide or protein. The amino acid can be a naturally occurring amino acid and, unless otherwise limited, may encompass known analogues of natural amino acids that can function in a similar manner as naturally occurring amino acids.

As used herein, “recombinant,” when used in connection with a peptide, polypeptide or protein, means a molecule that has been created or modified through deliberate human intervention such as by protein engineering. For example, a recombinant polypeptide is one having an amino acid sequence that has been modified to include an artificial amino acid sequence or to include some other amino acid sequence that is not present within its native/endogenous/non-recombinant form.

Further, a recombinant peptide, polypeptide or protein has a structure that is not identical to that of any naturally occurring peptide, polypeptide or protein. As such, a recombinant peptide, polypeptide or protein can be prepared by synthetic methods such as those known to one of skill in the art.

If, and when, modified substrate proteins are to be isolated, complete purification is not required. For example, the modified substrate proteins described herein can be isolated and purified from normally associated material in conventional ways, such that in the purified preparation, the proteins are the predominant species in the preparation. At the very least, the degree of purification is such that extraneous material in the preparation does not interfere with use of the proteins in the manner disclosed herein. The peptide, polypeptide or protein can be at least 80%, at least 85%, at least 90%, at least 91%, at least 92%, at least 93%, at least 94%, at least 95%, at least 96%, at least 97%, at least 98% or at least 99% pure. Alternatively stated, the polypeptide is substantially free of cellular material such that preparations of the polypeptide can contain less than 30%, 25%, 20%, 15%, 10%, 9%, 8%, 7%, 6%, 5%, 4%, 3%, 2%, or 1% (dry weight) of contaminating protein. When the polypeptide or an active variant or fragment thereof is recombinantly produced, culture medium represents less than 30%, 25%, 20%, 15%, 10%, 9%, 8%, 7%, 6%, 5%, 4%, 3%, 2%, or 1% (dry weight) of chemical precursors or non-protein-of-interest chemicals.

It is known in the art that amino acids within the same conservative group can typically substitute for one another without substantially affecting the function of a protein. For the purpose of the present disclosure, such conservative groups are set forth in Table 5 and are based on shared properties. See also, Alberts et al., “Small molecules, energy, and biosynthesis” 56-57 In: Molecular Biology of the Cell (Garland Publishing Inc. 3^{rd} ed. 1994).

The following six groups each contain amino acids that are typical, but not necessarily exclusive, conservative substitutions for one another: 1. Alanine (A), Serine (S), Threonine (T); 2. Aspartic acid (D), Glutamic acid (E); 3. Asparagine (N), Glutamine (Q); 4. Arginine (R), Lysine (K); 5. Isoleucine (I), Leucine (L), Methionine (M), Valine (V); and 6. Phenylalanine (F), Tyrosine (Y), Tryptophan (W).

Substantial changes in function of a peptide, polypeptide or protein can be made by selecting substitutions that are less conservative than those listed in the table above, that is, by selecting residues that differ more significantly in their effect on maintaining (a) the structure of the polypeptide backbone in the area of substitution, (b) the charge or hydrophobicity of the polypeptide at the target site, or (c) the bulk of a side chain. The substitutions that in general can be expected to produce the greatest changes in the polypeptide's properties will be those in which (a) a hydrophilic residue, for example, seryl or threonyl, is substituted by a hydrophobic residue, for example, leucyl, isoleucyl, phenylalanyl, valyl or alanyl; (b) a cysteine or proline is substituted by any other residue; (c) a residue having an electropositive side chain, for example, lysyl, arginyl or histidyl, is substituted by an electronegative side chain, for example, glutamyl or aspartyl; (d) a residue having a bulky side chain, for example, phenylalanyl, is substituted by a residue not having a side chain, for example, glycyl; or (e) by increasing the number of sulfation or glycosylation.

In one aspect, the present disclosure is directed to an isolated polypeptide encoded by the recombinant nucleic acid molecule comprising about 90% identity to an amino acid sequence selected from SEQ ID NO:10, SEQ ID NO:12, and SEQ ID NO:14, wherein the polypeptide is a substrate protein of a plant pathogen-specific protease. In another embodiment, the isolated polypeptide can comprise about 95% identity to an amino acid sequence selected from SEQ ID NO:10, SEQ ID NO:12, and SEQ ID NO:14, wherein the polypeptide is a substrate protein of a plant pathogen-specific protease. In other embodiments, the isolated polypeptide can comprise about 96% identity, about 97% identity, about 98% identity, about 99% identity, and even 100% identity to an amino acid sequence selected from SEQ ID NO:10, SEQ ID NO:12, and SEQ ID NO:14, wherein the polypeptide is a substrate protein of a plant pathogen-specific protease.

An example of a fusion protein (that is, a modified substrate protein of a pathogen-specific protease) therefore includes that of SEQ ID NO:10, SEQ ID NO:12, SEQ ID NO:14, SEQ ID NO: 41, SEQ ID NO: 44, SEQ ID NO: 47, SEQ ID NO: 50, SEQ ID NO: 53 and including polypeptides comprising about 95% identity, about 96% identity, about 97% identity, about 98% identity, about 99% identity and even 100% identity to an amino acid sequence selected from SEQ ID NO:10, SEQ ID NO:12, and SEQ ID NO:14.

In addition to the full-length amino acid sequence of the modified substrate protein of the pathogen-specific protease, it is intended that the modified substrate protein can be a fragment or variant thereof that is capable of being recognized by the plant pathogen protease and/or its corresponding NB-LRR protein. For amino acid sequences, “fragment” means a portion of the amino acid sequence of a reference polypeptide or protein. Fragments of an amino acid sequence may retain the biological activity of the reference polypeptide or protein. For example, less than the entire amino acid sequence of the modified substrate protein can be used and may have substrate protein activity and/or NB-LRR protein binding activity. Thus, fragments of the reference polypeptide or protein can be at least 150, 200, 250, 300, 350, 400 or 450 amino acid residues, or up to the number of amino acid residues present in a full-length modified substrate protein. For example, about 80 amino acids can be deleted from the N-terminus of PBS1 while retaining function. See, DeYoung et al. (2012), supra. Alternatively, about 100 amino acids can be deleted from the C-terminus of PBS1 while retaining function. Id.

Likewise, a “variant” peptide, polypeptide or protein means a substantially similar amino acid sequence to the amino acid sequence of a reference peptide, polypeptide or protein. For amino acid sequences, a variant comprises an amino acid sequence derived from a reference peptide, polypeptide or protein by deletion (so-called truncation) of one or more amino acids at the N-terminal and/or C-terminal end of the amino acid sequence of the reference; deletion and/or addition of one or more amino acids at one or more internal sites in the amino acid sequence of the reference; or substitution of one or more amino acids at one or more sites in the amino acid sequence of the reference. Variant peptides, polypeptides or proteins encompassed by the present disclosure are biologically active, that is, they continue to possess the desired biological activity of the reference peptide, polypeptide or protein as described herein. Such variants may result from, for example, genetic polymorphism or human manipulation. Biologically active variants will have at least 70%, 75%, 80%, 85%, 90%, 91%, 92%, 93%, 94%, 95%, 96%, 97%, 98%, 99% or more sequence identity to the amino acid sequence of the reference peptide polypeptide or protein as determined by sequence alignment programs and parameters described above. For example, a biologically active variant of a modified substrate protein may differ by as few as 1-15 amino acid residues, as few as 1-10, such as 6-10, as few as 5, as few as 4, 3, 2, or even 1 amino acid residue.

Deletions, insertions and substitutions of the modified substrate proteins are not expected to produce radical changes in the characteristics of the polypeptides. However, when it is difficult to predict the exact effect of the substitution, deletion or insertion in advance of doing so, one of skill in the art will appreciate that the effect can be evaluated by routine activity assays as described herein.

As above, variant peptides, polypeptides and proteins also encompass sequences derived from a mutagenic and recombinogenic procedure such as DNA shuffling. With such a procedure, one or more nucleic acid molecules can be manipulated to encode new modified substrate proteins possessing the desired properties. In this manner, libraries of recombinant nucleic acid molecules can be generated from a population of related nucleic acid molecules comprising sequence regions that have substantial sequence identity and can be homologously recombined in vitro or in vivo. For example, using this approach, sequence motifs encoding a domain of interest can be shuffled between the nucleic acid molecules identified by the methods described herein and other known substrate protein-encoding nucleic acid molecules to obtain a new nucleic acid molecule that encodes a modified substrate protein with an improved property such as increased activity or an expanded pH or temperature range. As such, a peptide, polypeptide or protein of the present disclosure can have many modifications.

The present disclosure therefore includes recombinant modified substrate proteins/fusion proteins, where the substrate proteins have heterologous protease recognition sequences, as well as active fragments or variants thereof.

### Transformed Plant Cells, Plant Parts and Plants

Compositions of the present disclosure also include transformed plant cells, plant parts and plants (i.e., subject plant cells, plant parts or plants) having a resistance to an increased number of plant pathogens when compared with control/native plant cells, plant parts or plants.

The transformed plant cells, plant parts or plants can have at least one nucleic acid molecule, nucleic acid construct, expression cassette or vector as described herein that encodes a modified substrate protein of a pathogen-specific protease, where the modified substrate protein has a heterologous protease recognition sequence.

As used herein, “subject plant cell,” “subject plant part” or “subject plant” means one in which a genetic alteration, such as transformation, has been effected as to a nucleic acid molecule of interest, or is a plant cell, plant part or plant that descended from a plant cell, plant part or plant so altered and that comprises the alteration.

As used herein, “control plant cell,” “control plant part” or “control plant” means a reference point for measuring changes in phenotype of the subject plant cell, plant part or plant. A control plant cell, plant part or plant can comprise, for example: (a) a wild-type plant cell, plant part or plant (i.e., of the same genotype as the starting material for the genetic alteration that resulted in the subject plant cell, plant part or plant); (b) a plant cell, plant part or plant of the same genotype as the starting material, but which has been transformed with a null construct (i.e., with a construct that has no known effect on the trait of interest, such as a construct comprising a marker gene); (c) a plant cell, plant part or plant that is a non-transformed segregant among progeny of a subject plant cell, plant part or plant; (d) a plant cell, plant part or plant genetically identical to the subject plant cell, plant part or plant, but which is not exposed to conditions or stimuli that would induce expression of the gene of interest; or (e) the subject plant cell, plant part or plant itself, under conditions in which the nucleic acid molecule/construct of interest is not expressed.

Methods of introducing nucleotide sequences into plants, plant parts or plant host cells are well known in the art and are discussed in greater detail below.

As used herein, “plant cell” or “plant cells” means a cell obtained from or found in seeds, suspension cultures, embryos, meristematic regions, callus tissue, leaves, roots, shoots, gametophytes, sporophytes, pollen and microspores. Plant cell also includes modified cells, such as protoplasts, obtained from the aforementioned tissues, as well as plant cell tissue cultures from which plants can be regenerated, plant calli and plant clumps.

As used herein, “plant part” or “plant parts” means organs such as embryos, pollen, ovules, seeds, flowers, kernels, ears, cobs, leaves, husks, stalks, stems, roots, root tips, anthers, silk and the like.

As used herein, “plant” or “plants” means whole plants and their progeny. Progeny, variants and mutants of the regenerated plants also are included, provided that they comprise the introduced nucleic acid molecule.

As used herein, “grain” means mature seed produced by commercial growers for purposes other than growing or reproducing the species. The class of plants that can be used in the methods described herein is generally as broad as the class of higher plants amenable to transformation techniques, including both monocotyledonous (monocots) and dicotyledonous (dicots) plants.

Examples of plant species of interest herein include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), Citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), Macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals, and conifers.

Vegetables of interest include, but are not limited to, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamentals of interest include, but are not limited to, azalea (Rhododendron spp.), hydrangea (Macrophylla hydrangea), Hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers of interest include, but are not limited to, pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In some instances, the plant cells, plant parts or plants of interest are crop plants (e.g., corn, alfalfa, sunflower, Brassica, soybean, cotton, safflower, peanut, sorghum, wheat, millet, tobacco, etc.).

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The present disclosure therefore includes transgenic plant cells, plant parts and plants having incorporated therein at least one nucleic acid molecule that encodes a modified substrate protein of a pathogen-specific protease, where the modified substrate protein has a heterologous protease sequence, to confer disease resistance to plant pathogens expressing specific proteases.

Methods of the present disclosure include introducing and expressing in a plant cell, plant part or plant a nucleic acid molecule or construct as described herein. As used herein, “introducing” means presenting to the plant cell, plant part or plant, a nucleic acid molecule or construct in such a manner that it gains access to the interior of a cell of the plant. The methods do not depend on the particular method for introducing the nucleic acid molecule or nucleic acid construct into the plant cell, plant part or plant, only that it gains access to the interior of at least one cell of the plant or plant part. Methods of introducing nucleotide sequences, selecting transformants and regenerating whole plants, which may require routine modification in respect of a particular plant species, are well known in the art. The methods include, but are not limited to, stable transformation methods, transient transformation methods, virus-mediated methods and sexual breeding. As such, the nucleic acid molecule or construct can be carried episomally or integrated into the genome of the host cell.

As used herein, “stable transformation” means that the nucleic acid molecule or construct of interest introduced into the plant integrates into the genome of the plant and is capable of being inherited by the progeny thereof. As used herein, “transient transformation” means that the nucleic acid molecule or construct of interest introduced into the plant is not inherited by progeny.

Methods of transforming plants and introducing a nucleotide sequence of interest into plants can and will vary depending on the type of plant, plant part or plant host cell (i.e., monocotyledonous or dicotyledonous) targeted for transformation. Methods of introducing nucleotide sequences into plant host cells therefore include Agrobacterium-mediated transformation (e.g., A. rhizogenes or A. tumefaciens; U.S. Pat. Nos. 5,563,055 and 5,981,840), calcium chloride, direct gene transfer (Paszkowski et al. (1984) EMBO J. 3:2717-2722), electroporation (Riggs et al. (1986) Proc. Natl. Acad. Sci. USA 83:5602-5606), microinjection (Crossway et al. (1986) Biotechniques 4:320-334), microprojectile bombardment/particle acceleration (McCabe et al. (1988) Biotechnology 6:923-926; and Tomes et al., “Direct DNA transfer into intact plant cells via microprojectile bombardment” In: Plant Cell, Tissue, and Organ Culture: Fundamental Methods (Gamborg & Phillips eds., Springer-Verlag 1995); as well as U.S. Pat. Nos. 4,945,050; 5,879,918; 5,886,244 and 5,932,782), polyethylene glycol (PEG), phage infection, viral infection, and other methods known in the art. See also, EP Patent Nos. 0 295 959 and 0 138 341.

A nucleic acid molecule or construct as described above herein can be introduced into the plant cell, plant part or plant using a variety of transient transformation methods. Methods of transiently transforming plant cells, plant parts or plants include, but are not limited to, Agrobacterium infection, microinjection or particle bombardment. See, Crossway et al. (1986) Mol. Gen. Genet. 202:179-185; Hepler et al. (1994) Proc. Natl. Acad. Sci. USA 91:2176-2180; Hush et al. (1994) J. Cell Sci. 107:775-784; and Nomura et al. (1986) Plant Sci. 44:53-58. Alternatively, the plant cell, plant part or plant can be transformed by viral vector systems or by precipitation of the nucleic acid molecule or construct in a manner that precludes subsequent release of the DNA. Thus, transcription from the particle-bound nucleotide sequence can occur, but the frequency with which it is released to become integrated into the genome is greatly reduced. Such methods include the use of particles coated with polyethylimine (PEI; Sigma; St. Louis, Mo.).

Likewise, the nucleic acid molecules or constructs as described herein can be introduced into the plant cell, plant part or plant by contacting it with a virus or viral nucleic acids. Generally, such methods involve incorporating the nucleic acid molecule or construct within a viral DNA or RNA molecule. It is recognized that the nucleotide sequences can be initially synthesized as part of a viral polyprotein, which later can be processed by proteolysis in vivo or in vitro to produce the desired recombinant protein. Methods for introducing nucleotide sequences into plants and expressing the protein encoded therein, involving viral DNA or RNA molecules, are well known in the art. See, Porta et al. (1996) Mol. Biotechnol. 5:209-221; as well as U.S. Pat. Nos. 5,866,785; 5,889,190; 5,889,191 and 5,589,367.

By way of example, in one embodiment, the SMV protease cleavage site (SEQ ID NO:2) is inserted into a GmPBS1 polyprotein sequence so that the modified PBS1 protein is produced as part of the full-length modified GmPBS1 sequence (e.g., SEQ ID NOs:10, 12, and 14).

Methods also are known in the art for the targeted insertion of a nucleic acid molecule or construct at a specific location in the plant genome. In some instances, insertion of the nucleic acid molecule or construct at a desired genomic location can be achieved by using a site-specific recombination system. See, Int'l Patent Application Publication Nos. WO 99/025821, WO 99/025854, WO 99/025840, WO 99/025855 and WO 99/025853.

Transformation techniques for monocots therefore are well known in the art and include direct gene uptake of exogenous nucleic acid molecules or constructs by protoplasts or cells (e.g., by PEG- or electroporation-mediated uptake, and particle bombardment into callus tissue). Transformation of monocots via Agrobacterium also has been described. See, Int'l Patent Application Publication No. WO 94/00977 and U.S. Pat. No. 5,591,616; see also, Christou et al. (1991) Bio/Technology 9:957-962; Datta et al. (1990) Bio/Technology 8:736-740; Fromm et al. (1990) Biotechnology 8:833-844; Gordon-Kamm et al. (1990) Plant Cell 2:603-618; Koziel et al. (1993) Bio/Technology 11:194-200; Murashige & Skoog (1962) Physiologia Plantarum 15:473-497; Shimamoto et al. (1989) Nature 338:274-276; Vasil et al. (1992) Bio/Technology 10:667-674; Vasil et al. (1993) Bio/Technology 11:1553-1558; Weeks et al. (1993) Plant Physiol. 102:1077-1084; and Zhang et al. (1988) Plant Cell Rep. 7:379-384; as well as EP Patent Application Nos. 0 292 435; 0 332 581 and 0 392 225; Int'l Patent Application Publication Nos. WO 93/07278 and WO 93/21335; and U.S. Pat. No. 7,102,057.

Transformation techniques for dicots also are well known in the art and include Agrobacterium-mediated techniques and techniques that do not require Agrobacterium. Non-Agrobacterium-mediated techniques include the direct uptake of exogenous nucleic acid molecules by protoplasts or cells (e.g., by PEG- or electroporation-mediated uptake, particle bombardment, or microinjection). See, Klein et al. (1987) Nature 327:70-73; Paszkowski et al. (1984) EMBO J. 3:2717-2722; Potrykus et al. (1985) Mol. Gen. Genet. 199:169-177; and Reich et al. (1986) Bio/Technology 4:1001-10041; as well as U.S. Pat. No. 7,102,057.

Plant cells that have been transformed can be grown into plants by methods well known in the art. See, McCormick et al. (1986) Plant Cell Rep. 5:81-84. These plants then can be grown, and either pollinated with the same transformed strain or different strains, and the resulting progeny having the desired phenotypic characteristic identified. Two or more generations can be grown to ensure that expression of the desired phenotypic characteristic is stably maintained and inherited, and then seeds harvested to ensure expression of the desired phenotypic characteristic has been achieved.

It has been shown that the produced plant cells and plants have enhanced confer resistance to disease. By way of example, by introducing a fusion protein (e.g., SEQ ID NO:10, SEQ ID NO:12, SEQ ID NO:14), including the modified GmPBS1 sequence into soybean, there is seen an enhanced resistance to infection of the soybean by soybean mosaic virus (SMV). Particularly, the excised PBS1 protein, although cleaved in its activation loop by NIa protease, appears to still be able to activate a native soybean resistance protein, which then prevents spread of SMV through the plant.

The present disclosure therefore provides methods of introducing into plants, plant parts and plant host cells the nucleic acid constructs described herein, for example, an expression cassette of the present disclosure, which encode a modified substrate protein of a pathogen-specific protease, where the substrate protein has a heterologous protease recognition sequence.

### EXAMPLES

The disclosure will be more fully understood upon consideration of the following non-limiting examples, which are offered for purposes of illustration, not limitation.

### Example 1

In this Example, modified GmPBS1 substrate proteins were generated and analyzed as substrates for soybean mosaic virus (SMV) protease.

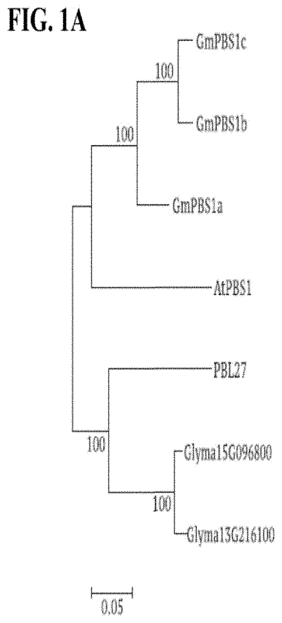

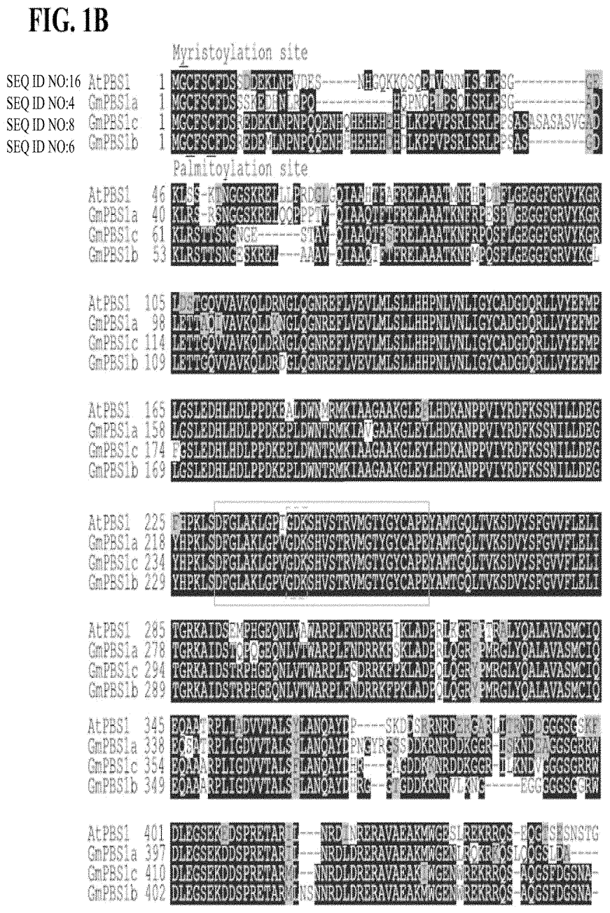

PBS1 is one of the most widely conserved defense genes in flowering plants. Using a bioinformatics approach, three genes within the Glycine max genome that encode proteins with significant amino acid homology to Arabidopsis PBS1 (AtPBS1; At5g13160) (SEQ ID NO:16) were identified (see FIGS. 1A & 1B). These were designated GmPBS1a (Glyma08g47570) (SEQ ID NO:3), GmPBS1b (Glyma10g44580) (SEQ ID NO:5), and GmPBS1c (Glyma20g39370) (SEQ ID NO:7). Phylogenetic analysis showed that all three GmPBS1 proteins clustered together and were more closely related to AtPBS1 than the next most similar gene to PBS1, PBL27. The three GmPBS1 orthologs contained several conserved domains present in AtPBS1, including conservation of the AvrPphB recognition motif within the activation segment and putative palmitoylation and myristoylation motifs for plasma membrane localization.

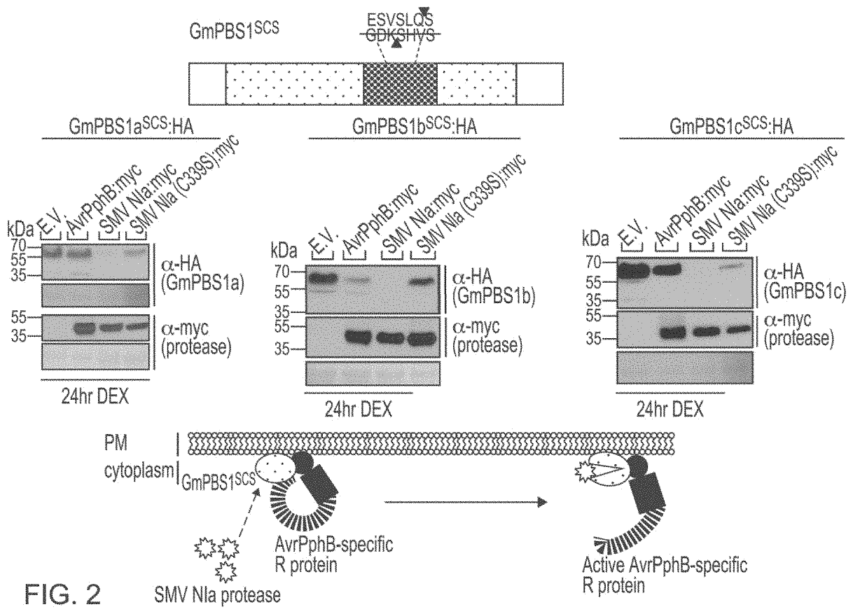

Since the amino acids at the AvrPphB cleavage site are conserved between AtPBS1 and the soybean PBS1 orthologs, it was hypothesized that modified soybean PBS1 substrate proteins could be engineered to function as ‘decoys’ for the SMV NIa protease. As shown in FIG. 2, using site-directed mutagenesis, the AvrPphB recognition motif Gly-Asp-Lys-Ser-His-Val-Ser (GDKSHVS) (SEQ ID NO:1) was replaced with a SMV NIa protease recognition motif Glu-Ser-Val-Ser-Leu-Gln-Ser (ESVSLQS) (SEQ ID NO:2), generating GmPBS1SCS derivatives. Transient coexpression of the modified GmPBS1 constructs along with the SMV NIa protease in N. benthamiana resulted in NIa-mediated cleavage of GmPBS1SCS. Collectively, these data suggested engineered modified soybean PBS1 proteins could function as substrates for the SMV NIa protease.

### Example 2

In this Example, recognition of AvrPphB in soybean was analyzed.

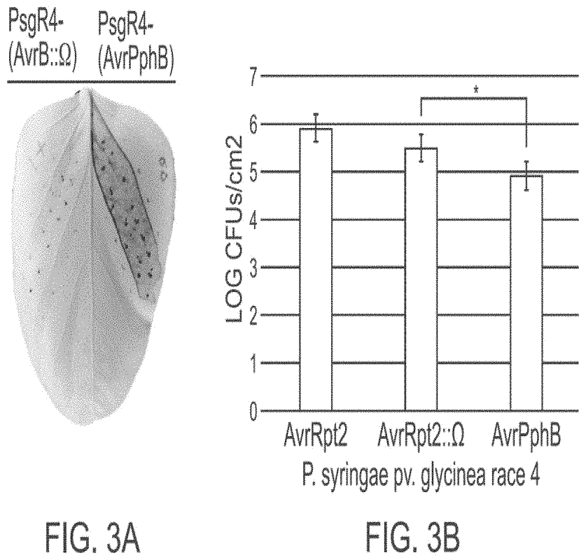

Engineering resistance to crop plant pathogens may not require transferring the modified Arabidopsis ‘decoy’ recognition system to a crop plant if the crop plant recognizes AvrPphB. To test whether soybean recognizes AvrPphB, P. syringae pv. glycinea expressing AvrPphB or AvrB::Ω (a non-functional effector used as an empty vector control) were infiltrated into a unifoliate leaf of soybean cultivar Flambeau. The leaf was removed from the plant 24 hours post-inoculation and the chlorophyll removed with hot 70% ethanol. P. syringae pv. glycinea expressing AvrPphB induced a hypersensitive response (HR) in soybean cv. flambeau, indicated by browning of the inoculated leaf panel (see FIGS. 3A & 3B). These data suggested soybean likely contains an endogenous R protein that recognizes cleavage of GmPBS1.

### Example 3

In this Example, the effect of AvrPphB-specific R protein activation on soybean mosaic virus (SMV) resistance was analyzed.

Soybean mosaic virus (SMV)-mediated expression of AvrPphB in soybean v. Flambeau triggers resistance to SMV. Green fluorescent protein (GFP), AvrPphB or AvrPphB (C98S) (an enzymatically-inactive derivative of AvrPphB) were transiently expressed in 10-12-day old seedlings by rub-inoculation, as previously described in Wang et al. (2006). Briefly, approximately three weeks post-inoculation (wpi), the third trifoliate leaflet was photographed and harvested. For immunoblot analysis, proteins (10 g) were fractionated on 4-20% SDS-PAGE gels and subjected to immunoblot analysis using α-GFP, α-AvrPphB or α-SMV-CP (SMV coat protein) specific antibodies.

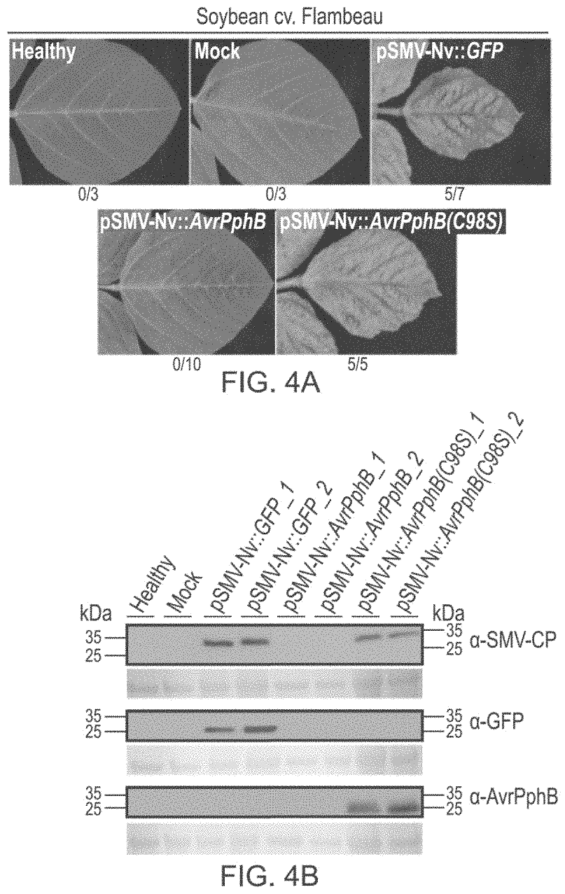

As shown in FIGS. 4A & 4B, insertion of AvrPphB into the modified SMV genome blocked symptom development and detectable SMV-CP (coat protein) accumulation in the upper, non-inoculated leaflets (FIG. 4A). This recognition of AvrPphB is dependent upon the protease activity because a protease inactive derivative of AvrPphB (AvrPphB(C98S)) failed to prevent systemic spread of SMV. These results demonstrated that activation of the soybean resistance protein that detects AvrPphB protease activity is sufficient to confer resistance to SMV.

### Example 4

In this Example, transgenic Arabidopsis expressing a modified derivative of PBS1 containing a TuMV NIa protease cleavage site was analyzed.

Prior work established that PBS1 (SEQ ID NO:16) could be engineered to function as a target for viral proteases, at least when transiently overexpressed in N. benthamiana. To test whether cleavage of modified PBS1 can activate RPS5 and initiate an effective immune response against TuMV, transgenic Arabidopsis was generated expressing a modified PBS1 substrate protein (PBS1TuMV) under the native promoter and terminator. The PBS1TuMV transgenic Arabidopsis was then infected with TuMV::GFP.

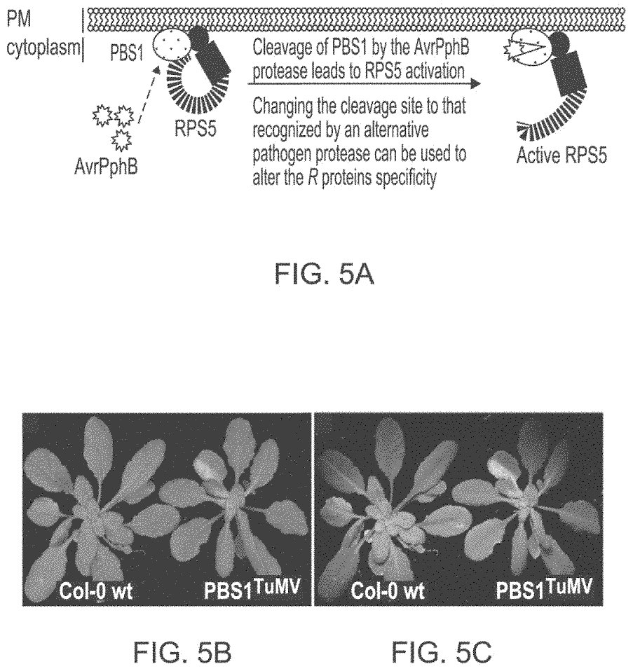

At 11 days post-infection, TuMV::GFP spread from the initial site of infection to newly emerging leaves of wild-type (nontransgenic) Arabidopsis. Interestingly, the PBS1TuMV transgenic Arabidopsis lines developed extensive chlorosis and necrosis (see FIGS. 5B & 5C). Systemic cell death in the PBS1TuMV transgenic lines correlated with a significant reduction in GFP fluorescence and TuMV accumulation. Collectively, these data suggested that RPS5 could be activated by cleavage of an engineered PBS1, and this activation significantly reduced virus accumulation.

### Example 5

In this Example, transgenic Arabidopsis expressing a modified derivative of PBS1 using a strong promoter was analyzed.

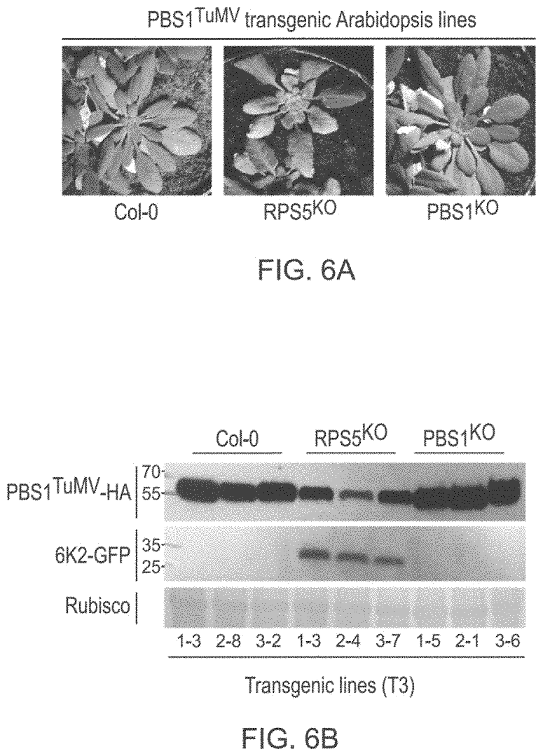

The method as used in Example 4 with the modification of placing the PBS1 gene under control of a strong constitutive promoter (cauliflower mosaic virus 35S promoter), allowing the modified PBS1 protein to accumulate to higher levels, was used. As shown in FIGS. 6A & 6B, transgenic Arabidopsis plants expressing PBS1^{TuMV} under a strong promoter displayed resistance to TuMV infection without trailing necrosis at 19 days after viral infection. Particularly, as shown in FIG. 6A, all plants were infected with a TuMV derivative that expressed green fluorescence protein (GFP) fused to the viral 6K2protein. Green fluorescence in the leaves indicates viral spread. The transgenic wild-type Col-0 pants and pbs1 null mutants (PBS1^{KO}) transformed with PBS1^{TuMV} showed no visible virus spread, whereas rps5 null mutants plants (RPS5^{KO}) showed systemic spread.

Additionally, total protein was isolated from the indicated transgenic lines and immunoblotted to assess levels of the PBS1^{TuMV} decoy protein (FIG. 6B, top row) and the virus 6K2:GFP protein (FIG. 6B, middle row). Each lane represents an independent transgenic line. No virus protein was detected in the wild-type and pbs1 mutant lines.

### Example 6

In this Example, it was investigated whether crop plants such as soybean, wheat and barley may already contain disease resistance proteins that are functionally equivalent to RPS5. If so, the PBS1 decoy system developed herein should be able to be modified to be used with these plants.

To assess whether wheat, barley and soybean contain disease resistance proteins functionally equivalent to RPS5, these species were analyzed for their ability to recognize the protease AvrPphB from Pseudomonas syringae. Hypersensitive resistance assays were performed by injecting leaves of the indicated plant varieties with P. syringae strains expressing wild-type AvrPphB or the protease inactive mutant of AvrPphB (C98S). The soybean leaf has been extracted with ethanol to remove chlorophyll, revealing brown phenolic deposits marking the zone of cell death.

As shown in FIG. 7A, it was found that most varieties (cultivars) of wheat, barley and soybean responded to AvrPphB with a strong cell death response (soybean and barley) or strong chlorotic response (wheat), both of which are indicative of defense activation, and these responses are dependent on protease activity as a protease inactive mutant of AvrPphB (C98S) did not induce cell death or chlorosis. For the barley leaves, infiltrated regions are marked with up and down brackets, with wild type AvrPphB indicated by a dot, and C98S marked with only the brackets.

Furthermore, PBS1 orthologs was isolated from soybean and barley. The indicated proteins were co-expressed in Nicotiana benthamiana and then analyzed using immunoblots and the indicated antibodies. It was demonstrated that the encoded PBS1 proteins were cleaved by AvrPphB (FIG. 7B). The boxed bands indicate cleavage products of PBS1.

These findings indicate that it should be possible to modify PBS1 proteins from diverse crop species to engineer novel resistance traits.

All of the patents, patent applications, patent application publications and other publications recited herein are hereby incorporated by reference to the extent they are consistent herewith.

### Example 7

Monocot PBS1 Decoy Proteins are Used to Engineer Disease Resistance in Crops. The PBS1 protein of Arabidopsis is a protein kinase that functions to regulate defense signaling in response to perception of pathogen-derived molecules by pattern recognition receptors, which are transmembrane proteins localized on the cell surface (Zhang et al., 2010). PBS1 was identified in 2001 (Swiderski and Innes, 2001). Subsequently, PBS1 was shown to be among the most highly conserved proteins in flowering plants (Caldwell and Michelmore, 2009), with clear orthologs present in both monocot and dicot plant species. As a central regulator of plant defense responses, PBS1 proteins from diverse plant species have been shown to be targeted by effector proteins from pathogens in order to suppress immune responses (Shao et al., 2003). Most plant species, however, have evolved a second layer of defense involving intracellular receptors that can detect effector-induced modification of PBS1. For example, in Arabidopsis, the intracellular receptor RPS5 monitors the status of PBS1 and activates a strong defense response upon proteolytic cleavage of PBS1 by the effector protease AvrPphB, which is injected into plant cells by the bacterial pathogen Pseudomonas syringae (Ade et al., 2007). AvrPphB also cleaves PBS1 proteins from soybean, barley and wheat, which strongly indicates that AvrPphB can cleave PBS1 proteins from all flowering plants. Importantly, AvrPphB induces a strong defense response in soybean, barley and wheat, which indicates that these crop species all contain intracellular immune receptors that are activated by PBS1 cleavage. In barley, the intracellular receptor PBR1 mediates recognition of AvrPphB protease activity. Collectively, these data indicate that most, if not all, crop plants contain intracellular immune receptors that activated by cleavage of PBS1, or close homologs.

The above summary focused on cleavage of PBS1 by AvrPphB. This protease cleaves PBS1 at a single site located in the activation loop of PBS1 (Shao et al., 2003), which is an exposed loop located between the conserved kinase motifs ‘DFG’ and ‘APE’ (between amino acids 231 and 261 of Arabidopsis PBS1). A seven amino sequence within this loop can be replaced with alternative sequences that then enable cleavage of PBS1 by proteases from other pathogens (Kim et al., 2016). Importantly, cleavage of these modified PBS1 proteins, which are referred to as ‘decoy PBS1’ proteins, still activates RPS5, and thus a strong immune response (Kim et al., 2016). Such decoy PBS1 proteins thus enable RPS5 to recognize proteases from most any pathogen and confer resistance to these pathogens. For example, RPS5 confers resistance to infection by Turnip mosaic virus (TuMV) when a cleavage site for the NIa protease from TuMV is inserted into the PBS1 activation loop (Kim et al., 2016).

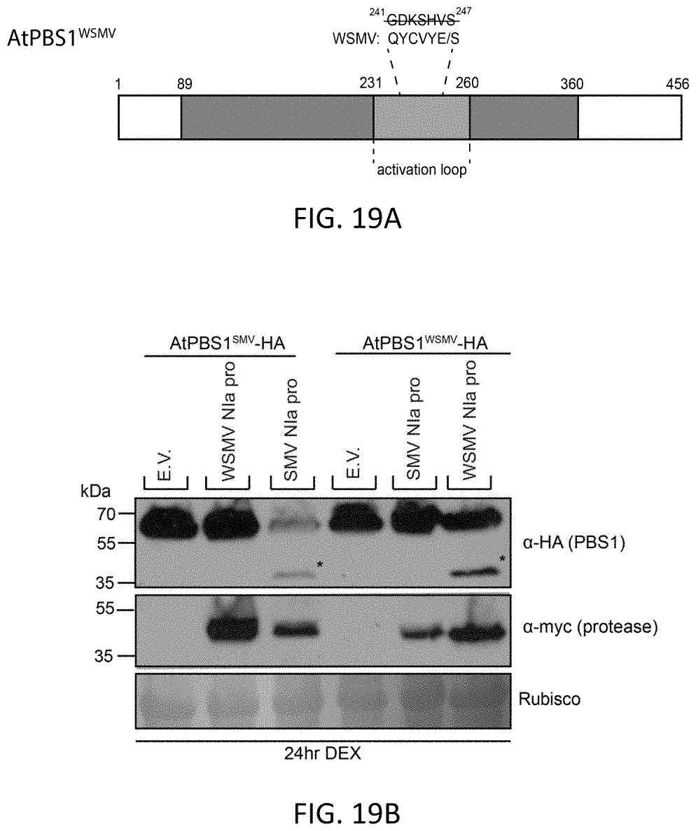

The PBS1 decoy approach has been extended to engineering novel disease resistance traits in crop plants using endogenous PBS1 genes of these species. For example, insertion of a cleavage sequence for the NIa protease from Soybean mosaic virus (SMV) into a soybean PBS1 protein confers resistance to infection by SMV. The present disclosure shows that PBS1 decoy engineering will also be effective in monocot crop species such as barley, wheat, rice, corn, and sorghum. The genomes of all of these crop species encode one or more copies of PBS1. The proteins encoded by these PBS1 genes are highly similar to each other (>80% amino acid sequence identity across the full length of the protein, and >95% identical within the kinase domain). The AvrPphB cleavage sites are conserved in all of them, and this disclosure shows that the PBS1 proteins from wheat and barley are cleaved by PBS1, and that AvrPphB induces immune responses in these species. Insertion of a protease cleavage site for the NIa protease from Wheat streak mosaic virus (WSMV) enables cleavage of PBS1 by this protease. Thus, it is straight forward to produce PBS1 decoy proteins from PBS1 proteins of any plant species, including monocot crops such as barley (SEQ ID NO:47), wheat (SEQ ID NO:44), rice (SEQ ID NO:50), corn (SEQ ID NO:41) and sorghum (SEQ ID NO:53). PBS1 decoy proteins are useful to engineer novel disease resistance traits in all monocot crops, where ‘PBS1 decoy’ is defined as a PBS1 decoy protein containing an altered amino acid sequence within the PBS1 activation loop that enables cleavage by proteases from any plant pathogen. FIG. 19A-19B shows examples of PBS1 decoy proteins that enable cleavage by WSMV NIa protease. Cleavage sequences for other proteases, including proteases produced by nematodes, fungi, and oomycetes, are substituted by following the methods disclosed herein.

The present disclosure has been described in connection with what are presently considered to be the most practical and preferred embodiments. However, the present disclosure has been presented by way of illustration and is not intended to be limited to the disclosed embodiments. Accordingly, one of skill in the art will realize that the present disclosure is intended to encompass all modifications and alternative arrangements of the compositions and methods as set forth in the appended claims.

### Materials and Methods

U.S. Pat. No. 9,816,102 (incorporated by reference) provides materials and methods modified herein; the modification is that the present application uses barley and wheat PBS1 genes rather than Arabidopsis PBS1, and introduces cleavage sites into PBS1 that enable cleavage by fungal proteases.

Plant Material and Growth Conditions

Barley seeds were planted in Cornell mix soil (1.2 cubic yards of mix contains 10.6 cubic feet of compressed peat moss, 20 lb of dolomitric limestone, 6 lb of 11-5-11 fertilizer, 12 cubic ft of vermiculite) in plastic pots. Barley plants were grown in a growth room on a 16 hr light/8 hr dark cycle with cool white fluorescent lights (85 to 112 μmol/m²/s at soil level) at 22° C. Plants were watered as needed to keep soil damp.

N. benthamiana seeds were sown in plastic pots containing Pro-Mix B Biofungicide potting mix supplemented with Osmocote slow-release fertilizer (14-14-14) and grown under a 12 hr photoperiod at 22° C. in growth rooms with average light intensities at plant height of 150 μEinsteins/m²/s.

Seed for wheat (Triticum aestivum subsp. aestivum) cultivars were ordered from the U.S. Department of Agriculture Wheat Germplasm Collection via the National Plant Germplasm System Web portal (https://www.ars-grin.gov/npgs/) or provided by S. Hulbert (Washington State University). Wheat plants were grown in clay pots containing Pro-Mix B Biofungicide potting mix supplemented with Osmocote slow-release fertilizer (14-14-14) and grown under a 12 hr photoperiod at 22° C. in growth rooms with average light intensities at plant height of 150 μEinsteins/m²/s.

P. syringae DC3000(D36E) in Planta Assays

Previously generated plasmids pVSP61-AvrPphB and pVSP61-AvrPphB(C98S) (a catalytically inactive mutant) (Simonich and Innes, 1995; Shao et al., 2003) were each transformed into D36E, a strain of Pseudomonas syringae pv. tomato DC3000 with 36 effectors removed (Wei et al., 2015). Bacteria were grown on King's media B (KB), supplemented with 50 μg of kanamycin per milliliter, for two days at 28° C., then suspended in 10 mM MgCl₂ to an OD₆₀₀ of 0.5. Suspensions were infiltrated into the underside of the primary leaf of 10-day old barley seedlings by needleless syringe. Each leaf was infiltrated with bacteria expressing AvrPphB and bacteria expressing AvrPphB(C98S), and the infiltrated areas were marked with permanent marker. Infiltrated leaves were checked for cell collapse two days post infiltrations, then photographed and phenotyped for chlorosis and necrosis five days post infiltrations.

For wheat inoculations, bacteria were grown and prepared in the same way, but the adaxial side of the second leaf of 14-day old wheat seedlings was infiltrated at three spots with one of the strains of bacteria per leaf. Responses were photographed three days after infiltration using a high intensity long-wave (365 nm) ultraviolet lamp (Black-Ray B-100AP, UVP, Upland, CA).

Phylogenetic Analyses

Homology searches were performed using BLASTp to gather barley amino acid sequences homologous to Arabidopsis PBS1 and PBS1-like proteins. First, AtPBL (1 to 27), BIK1, and other PBS1-homologous sequences were gathered by searching the Arabidopsis genome (TAIR10, GCA_000001735.1) with the AtPBS1 (OAO91748.1) amino acid sequence and by name search. Potential barley PBLs were collected by searching the barley protein database (assembly Hv_IBSC_PGSB_v2) with each Arabidopsis homologue and taking the top five hits derived from distinct genes.

For NLR phylogenetic analysis, the NB-ARC domain was extracted by NLR-parser (Steuernagel et al., 2015). For genes where no NB-ARC domain was automatically found, the upstream nucleotide sequence in the genome was inspected using BLASTx to look for fragments encoding an NB-ARC domain or CC domain. CC domains were identified by analyzing each predicted NLR with the BLAST Conserved Domain Search or by comparison to the CC domain in RPS5 for domains lacking the EDVID motif (SEQ ID NO: 38) (Marchler-Bauer and Bryant, 2004).

Nucleotide or amino acid sequences were aligned with Clustal Omega (Sievers et al., 2011). Bayesian phylogenetic trees were generated for the collected sequences using the program MrBayes under a mixed amino acid model (Ronquist et al., 2012). Parameters for the Markov chain Monte Carlo method were; nruns=2, nchains=2, diagnfreq=1000, diagnstat=maxstddev. The number of generations (ngen) was initially set at 200,000 and increased by 100,000 until the max standard deviation of split frequencies was below 0.01, or until it was below 0.05 after 1,000,000 generations. Phylogenetic trees were visualized in FigTree v1.4.3.

For the analysis of Pbr1 alleles, nucleotide sequences were selected from each sequenced allele that spanned from the start codon to the stop codon of the Rasmusson allele, including the intron. Sequences were aligned with Clustal Omega and then used to construct Neighbor-Joining trees in MEGA7 (Kumar et al., 2016). A bootstrap test of 1000 replicates was applied.

Genome Wide Association Study

The University of Minnesota Spring Barley Nested Association Mapping (NAM) population comprises 6,161 RILs generated from the variety Rasmusson crossed to 88 diverse parents that represent 99.7% of captured SNP diversity. In total, ˜24,000 SNPs were generated through use of genotyping by sequencing and the barley iSelect 9K SNP chip. The 89 parental lines were assayed for AvrPphB response as part of the initial survey of barley lines. Because the common parent, Rasmusson, displayed a strong hypersensitive response, NAM families derived from Rasmusson and a parent showing no response were chosen for GWAS.

Plants were assayed as described above using infiltrations of two Pseudomonas strains expressing either AvrPphB or AvrPphB(C98S). Phenotypes for at least six plants of each recombinant inbred line (RIL) were recorded as 0 (no response/low chlorosis) or 1 (hypersensitive reaction) depending on the parental phenotype they exhibited. Lines that showed phenotypic segregation between individuals were not included in the analysis.

Genome wide association analysis was performed with the gwas2 function from the R/NAM (Nested Association Mapping) package, which uses an empirical Bayesian framework to determine likelihood ratios for each marker (Xavier et al., 2015). Lines from each family were identified within a family vector to account for population stratification. Markers with a minor allele frequency below 0.05 or missing data of more than 20% were removed using the snpQC function prior to analysis. A threshold of 0.05 for the false discovery rate was used to identify significant associations. NLR-encoding gene prediction was generated using NLR-parser (Steuernagel et al., 2015) and the high confidence Morex barley genome protein predictions (Mascher et al., 2017).

For genetic fine mapping, eighteen additional RILs with recombination events in the GWAS interval were selected from other families that also had an AvrPphB-non-responding parent. To determine which RILs to select, we subset the master SNP file by family and removed SNPs that were not variable between Rasmusson and the other parent. For visualization, SNPs that did not match neighboring markers across RILs were assumed to be miscalls and were also removed; while these could indicate double recombination events, the probability for a double recombination occurring within the 22.65 Mb interval is 0.001, and would be even less between two or three SNPs.

Construction of Transgene Expression Plasmids

The AvrPphB:myc, AvrPphB(C98S):myc, RPS5:sYFP, and AtPBS1:HA constructs have been described previously (Shao et al., 2003; Ade et al., 2007; DeYoung et al., 2012). HORVU2Hr1G070690 (HvPbs1-1) and HORVU3Hr1G035810 (HvPbs1-2) were PCR amplified from barley accession CI 16151 (Manchuria background) and Rasmusson cDNA, respectively. The resulting fragments were gel-purified, using the QIAquick gel extraction kit (Qiagen), and cloned into the Gateway entry vector pCR8/GW/TOPO (Invitrogen) to generate pCR8/GW/TOPO:HORVU2Hr1G070690 and pCR8/GW/TOPO:HORVU3Hr1G035810, which we then designated pCR8/GW/TOPO:HvPbs1-1 and pCR8/GW/TOPO:HvPbs1-2, respectively.

The following genes were PCR amplified with attB-containing primers from the corresponding templates: HvPbs1-1 from pCR8/GW/TOPO:HvPbs1-1, HvPbs1-2 from pCR8/GW/TOPO:HvPbs1-2, Pbr1.b (HORVU3Hr1G107310) and Goi2 (HORVU3Hr1G109680) from Rasmusson cDNA, Pbr1.c from CI 16151 gDNA, LTI6b from Arabidopsis thaliana gDNA (Col-0), and NbPbs1 (Niben101Scf02996g03008.1) from Nicotiana benthamiana cDNA. The resulting PCR products were gel-purified, using the QIAquick gel extraction kit (Qiagen) or the Monarch DNA gel extraction kit (NEB), and recombined into the Gateway donor vectors pBSDONR(P1-P4) or pBSDONR(P4r-P2) using the BP Clonase II kit (Invitrogen) (Qi et al., 2012). The resulting constructs were sequence-verified to check for proper sequence and reading frame.

To generate protein fusions with the desired C-terminal epitope tags, pBSDONR(P1-P4): HvPbs1-1, pBSDONR(P1-P4):HvPbs1-2, and pBSDONR(P1-P4):NbPbs1 were mixed with the pBSDONR(P4r-P2):3×HA construct and the Gateway-compatible expression vector pBAV154 in a 2:2:1 molar ratio. A derivative of the destination vector pTA7001, pBAV154, carries the dexamethasone inducible promoter (Aoyama and Chua, 1997; Vinatzer et al., 2006). The pBSDONR(P1-P4):Pbr1.b and pBSDONR(P1-P4):Pbr1.c constructs were mixed with the pBSDONR(P4r-P2):sYFP construct and pBAV154 in a 2:2:1 molar ratio. The pBSDONR(P4r-P2):sYFP and pBSDONR(P4r-P2):3×HA constructs have been described previously (Qi et al., 2012). To generate the sYFP:LTI6b fusion protein, the pBSDONR(P4r-P2):LTI6b construct was mixed with the pBSDONR(P1-P4):sYFP construct and pBAV154 in a 2:2:1 molar ratio. Plasmids were recombined by the addition of LR Clonase II (Invitrogen) and incubated overnight at 25° C. following the manufactures instructions. Constructs were sequence verified and subsequently used for transient expression assays in N. benthamiana.

Transient Expression Assays in N. benthamiana

For transient expression assays in N. benthamiana, we followed the protocol described by DeYoung et al. (2012) and Kim et al. (2016). Briefly, the dexamethasone-inducible constructs were transformed into Agrobacterium tumefaciens GV3101 (pMP90) strains and were streaked onto Luria-Bertani (LB) plates containing 30 μg of gentamicin sulfate per milliliter and 50 μg of kanamycin per milliliter. Cultures were prepared in liquid LB media (5 ml) supplemented with 30 μg of gentamicin per milliliter and 50 μg of kanamycin per milliliter and shaken overnight at 30° C. and 250 rpm on a New Brunswick orbital shaker. After overnight culture, the bacterial cells were pelleted by centrifuging at 3000×g for 3 minutes and resuspended in 10 mM MgCl₂ supplemented with 100 μM acetosyringone (Sigma-Aldrich). The bacterial suspensions were adjusted to an OD₆₀₀ of 0.9 for HR and electrolyte leakage assays and an OD₆₀₀ of 0.3 for immunoprecipitation and immunoblotting assays, and incubated for 3 hours at room temperature. For co-expression of multiple constructs, suspensions were mixed in equal ratios. Bacterial suspension mixtures were infiltrated by needleless syringe into expanding leaves of 3-week-old N. benthamiana. Leaves were sprayed with 50 μM dexamethasone 45 hours after injection to induce transgene expression. Samples were harvested 6 hours after dexamethasone application for protein extraction, flash-frozen in liquid nitrogen, and stored at −80° C. HR was evaluated and leaves photographed 24 hours after dexamethasone application using a high intensity long-wave (365 nm) ultraviolet lamp (Black-Ray B-100AP, UVP, Upland, CA).

Immunoblot Analysis

Frozen N. benthamiana leaf tissue (0.5 g) was ground in two volumes of protein extraction buffer (150 mM NaCl, 50 mM Tris [pH 7.5], 0.1% Nonidet P-40 [Sigma-Aldrich], 1% plant protease inhibitor cocktail [Sigma-Aldrich], and 1% 2,2′-dipyridyl disulfide [Chem-Impex]) using a ceramic mortar and pestle and centrifuged at 10,000×g for 10 minutes at 4° C. to pellet debris. Eighty microliters of total protein lysate were combined with 20 μl of 5×SDS loading buffer, and the mixture was boiled at 95° C. for 10 minutes. All samples were loaded on a 4-20% gradient Precise™ Protein Gels (Thermo Fisher Scientific, Waltham, Mass.) and separated at 185 V for 1 hour in 1×Tris/Glycine/SDS running buffer. Total proteins were transferred to a nitrocellulose membrane (GE Water and Process Technologies, Trevose, Pa.). Ponceau staining was used to confirm equal loading of protein samples and successful transfer. Membranes were washed with 1×Tris-buffered saline (TBS; 50 mM Tris-HCl, 150 mM NaCl, pH 7.5) solution containing 0.1% Tween 20 (TBST) and blocked with 5% Difco™ Skim Milk (BD, Franklin Lakes, N.J.) overnight at 4° C. Proteins were detected with 1:5,000 diluted peroxidase-conjugated anti-HA antibody (rat monoclonal, Roche, catalog number 12013819001) and a 1:5,000 diluted peroxidase-conjugated anti-c-Myc antibody (mouse monoclonal, Thermo Fisher Scientific, catalog number MA1-81357) for 1 hour and washed three times for 10 minutes in TBST solution. Protein bands were imaged using an Immuno-Star™ Reagents (Bio-Rad, Hercules, Calif.) and X-ray film.

Allele Sequencing and Expression Analysis

DNA was isolated from ground frozen leaf tissue using the GeneJET Plant Genomic DNA Purification Kit (Thermo Scientific™). Primers were designed throughout the genes of interest and fragments were amplified from genomic DNA using Q5 2× Master Mix (NEB), then Sanger sequenced at the Cornell Biotechnology Resource Center. RNA was isolated from the primary leaf of a 10-day old plant using the RNeasy Plant Mini Kit (QIAGEN) after freezing and grinding. RNA samples were quantified using a NanoDrop™ spectrophotometer (Thermo Scientific™) and 500 ng of RNA from each sample were used to make cDNA with SuperScript III Reverse Transcriptase (Invitrogen) and oligo dT primers. DreamTaq™ DNA Polymerase (Thermo Scientific™) was used for 30-cycle PCRs of 1 μl of cDNA or 50 ng of gDNA template. Eight microliters of the PCR products were then visualized in a 1% agarose gel. Samples chosen for expression and sequence analysis were done so based on NAM population parent lines and to encompass two or more lines for all phenotypes.

Electrolyte Leakage Assays in N. benthamiana

Electrolyte leakage assays were performed as described previously (Kim et al., 2016). In brief, after infiltration of Agrobacterium strains into N. benthamiana, leaf discs were collected from the infiltrated area using a cork borer (5 mm diameter) 2 h post dexamethasone application. Four leaf discs from four individual leaves of four different plants were included for each replication. The leaf discs were washed three times with distilled water and floated in 5 ml of distilled water supplemented with 0.001% Tween 20 (Sigma-Aldrich). Conductivity was monitored using a Traceable Pen Conductivity Meter (VWR) at the indicated time points after dexamethasone induction.

Immunoprecipitation Assay in N. benthamiana

Frozen N. benthamiana leaf tissue (four leaves) was ground in 1 ml of IP buffer (50 mM Tris-HCl [pH 7.5], 150 mM NaCl, 10% Glycerol, 1 mM DTT, 1 mM EDTA, 1% NP40, 0.1% Triton X-100, 1% plant protease inhibitor cocktail [Sigma-Aldrich], and 1% 2,2′-dipyridyl disulfide [Chem-Impex]) using a ceramic mortar and pestle and gently rotated for 1 hour at 4° C. The samples were centrifuged at 10,000×g for 10 minutes at 4° C. twice to remove plant debris. Five hundred microliters of the clarified extract were then incubated with 10 μl of GFP-Trap A (Chromotek) α-GFP bead slurry overnight at 4° C. with constant end-over-end rotation. After overnight incubation, the α-GFP beads were pelleted by centrifugation at 4000×g for 1 minute at 4° C. and washed five times with 500 μl of IP wash buffer. Eighty microliters of the immunocomplexes were resuspended in 20 μl of 5×SDS loading buffer, and the mixture was boiled at 95° C. for 10 minutes. All protein samples were resolved on a 4-20% gradient Precise™ Protein Gels (Thermo Scientific, Waltham, Mass.) and separated at 185V for 1 hour in 1×Tris/Glycine/SDS running buffer. Total proteins were transferred to a nitrocellulose membrane (GE Water and Process Technologies, Trevose, Pa.). Membranes were blocked with 5% Difco™ Skim Milk (BD, Franklin Lakes, N.J.) overnight at 4° C. Proteins were detected with 1:5,000 horseradish peroxidase conjugated anti-HA antibody (rat monoclonal, Roche, catalog number 12013819001) or 1:5,000 monoclonal mouse anti-GFP antibody (Novus Biologicals, Littleton, Colo., catalog number NB600-597), washed in X Tris-buffered saline (TBS; 50 mM Tris-HCl, 150 mM NaCl, pH 7.5) solution containing 0.1% Tween 20 (TBST) overnight and incubated with 1:5,000 horseradish peroxidase-conjugated goat anti-mouse antibody (abcam, Cambridge, Mass. catalog number ab6789). The nitrocellulose membranes were washed three times for 15 minutes in TBST solution and protein bands were imaged using an Immuno-Star™ Reagents (Bio-Rad, Hercules, Calif.) or Supersignal® West Femto Maximum Sensitivity Substrates (Thermo Scientific, Waltham, Mass.) and X-ray film.

DAB Assay for Hydrogen Peroxide Accumulation in Wheat

Hydrogen peroxide accumulation was detected following the protocol described by Liu et al. (2012) and Thordal-Christensen et al. (1997). In brief, 0.01 g of DAB powder (Sigma-Aldrich) was dissolved in 10 ml of distilled water (pH 3.6) and incubated at 37° C. for 1 hour on a New Brunswick orbital shaker to dissolve the DAB powder. Wheat leaf segments were harvested from the infiltrated leaves 3 days post inoculation, (10 plants per treatment, experiment performed twice), immersed immediately in DAB solution and vacuum infiltrated for 10 seconds. The samples were wrapped in aluminum foil and incubated overnight in the dark. After overnight incubation, the stained leaf tissue was gently rinsed with distilled water, submerged in 70% ethanol and incubated at 70° C. to clear the chlorophyll. The cleared leaves were rinsed and stored in a lactic acid/glycerol/H₂O solution (1:1:1, v/v/v) for photography. Wheat leaves inoculated with 10 mM MgCl₂ (mock) or P. syringae DC3000(D36E) expressing AvrPphB(C98S) were used as controls.

TABLE 1
Redundancy in Genetic Code.

| Residue | Triplet Codons Encoding the Residue |
|---|---|
| Ala (A) | GCU, GCC, GCA, GCG |
| Arg (R) | CGU, CGC, CGA, CGG, AGA, AGG |
| Asn (N) | AAU, AAC |
| Asp (D) | GAU, GAC |
| Cys (C) | UGU, UGC |
| Gln (Q) | CAA, CAG |
| Glu (E) | GAA, GAG |
| Gly (G) | GGU, GGC, GGA, GGG |
| His (H) | CAU, CAC |
| Ile (I) | AUU, AUC, AUA |
| Leu (L) | UUA, UUG, CUU, CUC, CUA, CUG |
| Lys (K) | AAA, AAG |
| Met (M) | AUG |
| Phe (F) | UUU, UUC |
| Pro (P) | CCU, CCC, CCA, CCG |
| Ser (S) | UCU, UCC, UCA, UCG, AGU, AGC |
| Thr (T) | ACU, ACC, ACA, ACG |
| Trp (W) | UGG |
| Tyr (Y) | UAU, UAC |
| Val (V) | GUU, GUC, GUA, GUG |
| START | AUG |
| STOP | UAG, UGA, UAA |

Table 2. Responses of 150 Barley Lines when Infiltrated with Pseudomonas syringae DC3000(D36E) Expressing AvrPphB.

A catalytically inactive AvrPphB(C98S) mutant was used as a negative control and never elicited a response. Lines were scored as no response (N), low chlorosis (LC), chlorosis (C), high chlorosis (HC), and hypersensitive reaction (HR). Responses of 193 RILs from the UMN Spring Barley Nested Association Mapping (NAM) population when infiltrated with Pseudomonas syringae DC3000(D36E) expressing AvrPphB. The inactive protease AvrPphB(C98S) never elicited a response. Response phenotypes were used in genome wide association analysis. Hypersensitive reaction=1; no response or low chlorosis=0

TABLE 2
Responses of diverse barley lines when
infiltrated with Pseudomonas syringae
DC3000(D36E) expressing AvrPphB.

| Barley Line | Response |
|---|---|
| CIho4050 | HR |
| Haruna Nijo | HR |
| Kamet Mugi (CI2253) | HR |
| PI371817 | HR |
| PI640095 | HR |
| Rasmusson | HR |
| CIho14216 | HC |
| CIho14228 | HC |
| CIho14258 | HC |
| Gorak (PI41157) | HC |
| PI048133 | HC |
| PI061533 | HC |
| PI135758 | HC |
| PI410451 | HC |
| PI410483 | HC |
| PI531986 | HC |
| PI573615 | HC |
| PI573878 | HC |
| PI640226 | HC |
| CIho10034 | C |
| CIho2367 | C |
| CIho4214 | C |
| CIho4264 | C |
| Hv586 | C |
| PI057089 | C |
| PI071075 | C |
| PI119925 | C |
| PI129482 | C |
| PI163409 | C |
| PI174431 | C |
| PI223883 | C |
| PI447100 | C |
| PI467733 | C |
| PI584977 | C |
| PI640117 | C |
| PI640286 | C |
| CI 16139 (Mlg) | LC |
| CI 16141 (Mlh) | LC |
| CI 16143 (Mlk) | LC |
| CI 16145 (Mlp) | LC |
| CI 16147 (Mla7) | LC |
| CI 16149 (Mla10) | LC |
| C I16151 (Mla6) | LC |
| CI 16153 (Mla15) | LC |
| CI 16155 (Mla13) | LC |
| CIho15600 | LC |
| CIho2205 | LC |
| CIho6020 | LC |
| CIho7247 | LC |
| HOR11358 (Mla9) | LC |
| Hv545 | LC |
| Hv612 (PI371383) | LC |
| PI069521 | LC |
| PI190790 | LC |
| PI320217 | LC |
| PI328052 | LC |
| PI402037 | LC |
| PI452421 | LC |
| PI467758 | LC |
| PI640265 | LC |
| WBDC 028 | LC |
| WBDC 150 | LC |
| WBDC 173 | LC |
| WBDC 209 | LC |
| CI5541a (PI94828) | LC |
| Barke (HOR13170) | N |
| Baronesse (PI568246) | N |
| BCD47 | N |
| CI 16137 (Mla1) | N |
| CIho13743 | N |
| CIho14052 | N |
| CIho14319 | N |
| CIho14881 | N |
| CIho15349 | N |
| CIho15362 | N |
| CIho2542 | N |
| CIho4184 | N |
| CIho6294 | N |
| Columbia (PI494520) | N |
| Diamond (PI491573) | N |
| Duplex (PI290343) | N |
| F4 (SM127/Morex) | N |
| Golden Promise (PI343079) | N |
| Harrington | N |
| Hv501 (PI371259) | N |
| Hv531 (PI371293) | N |
| HV587 (PI371356) | N |
| Hv602 (PI371373) | N |
| HV644 (PI371481) | N |
| Manchuria (CI 2330) | N |
| Minn. Cross 299 (CI2208) | N |
| Minsturdi (CI1556) | N |
| Morex (CI15773) | N |
| NGB9601 | N |
| Pallas (PI265965) | N |
| PC11 (PI584763) | N |
| PC249 (PI584765) | N |
| PC84 (PI584764) | N |
| PI039590 | N |
| PI054915 | N |
| PI078609 | N |
| PI087844 | N |
| PI094875 | N |
| PI173518 | N |
| PI282616 | N |
| PI296460 | N |
| PI298708 | N |
| PI327680 | N |
| PI327859 | N |
| PI328155 | N |
| PI328485 | N |
| PI328577 | N |
| PI328632 | N |
| PI329000 | N |
| PI356719 | N |
| PI362207 | N |
| PI382313 | N |
| PI382860 | N |
| PI386650 | N |
| PI387098 | N |
| PI392524 | N |
| PI415348 | N |
| PI434794 | N |
| PI449279 | N |
| PI531896 | N |
| PI531917 | N |
| PI584786 | N |
| PI640220 | N |
| PI640376 | N |
| Sultan 5 (Mla12) | N |
| WBDC 016 | N |
| WBDC 020 | N |
| WBDC 032 | N |
| WBDC 035 | N |
| WBDC 042 | N |
| WBDC 061 | N |
| WBDC 082 | N |
| WBDC 092 | N |
| WBDC 103 | N |
| WBDC 115 | N |
| WBDC 142 | N |
| WBDC 172 | N |
| WBDC 213 | N |
| WBDC 227 | N |
| WBDC 234 | N |
| WBDC 292 | N |
| WBDC 302 | N |
| WBDC 336 | N |
| WBDC 348 | N |
| WBDC 350 | N |

HR—hypersensitive reaction
HC—high chlorosis
C—chlorosis
LC—low chlorosis
N—no response

Table 3. Responses of 193 RILs from the UMN Spring Barley Nested Association Mapping (NAM) Population when Infiltrated with Pseudomonas syringae DC3000(D36E) Expressing AvrPphB.

The inactive protease AvrPphB(C98S) never elicited a response. Response phenotypes were used in genome wide association analysis. Hypersensitive reaction=1; no response or low chlorosis=0. Responses of wheat varieties to P. syringae DC3000(D36E) expressing AvrPphB. The second leaves of 14-day old wheat seedlings were inoculated with P. syringae DC3000(D36E) carrying AvrPphB (OD600=0.5) by infiltration with a needleless syringe. Wheat responses were scored as no response, low chlorosis, or chlorosis three days post-inoculation. Wheat varieties were obtained from the U.S. Department of Agriculture Wheat Germplasm Collection or generously provided by Scot Hulbert (Washington State University)

TABLE 3
Responses of 193 RILs from the UMN
Spring Barley Nested Association Mapping
(NAM) population when infiltrated with
Pseudomonas syringae DC3000(D36E)
expressing AvrPphB.

| Barley Line | Response |
|---|---|
| HR656S001 | 0 |
| HR656S002 | 1 |
| HR656S003 | 1 |
| HR656S004 | 0 |
| HR656S005 | 1 |
| HR656S006 | 0 |
| HR656S007 | 0 |
| HR656S008 | 1 |
| HR656S010 | 1 |
| HR656S011 | 0 |
| HR656S012 | 1 |
| HR656S013 | 1 |
| HR656S014 | 0 |
| HR656S015 | 1 |
| HR656S016 | 0 |
| HR656S017 | 1 |
| HR656S018 | 0 |
| HR656S019 | 1 |
| HR656S020 | 0 |
| HR656S021 | 0 |
| HR656S022 | 1 |
| HR656S023 | 0 |
| HR656S024 | 0 |
| HR656S025 | 0 |
| HR656S026 | 0 |
| HR656S027 | 0 |
| HR656S028 | 1 |
| HR656S029 | 0 |
| HR656S030 | 1 |
| HR656S031 | 1 |
| HR656S033 | 1 |
| HR656S034 | 0 |
| HR656S035 | 1 |
| HR656S036 | 1 |
| HR656S037 | 0 |
| HR656S038 | 0 |
| HR656S039 | 0 |
| HR656S041 | 1 |
| HR656S042 | 0 |
| HR656S043 | 0 |
| HR656S045 | 0 |
| HR656S046 | 1 |
| HR656S048 | 1 |
| HR656S049 | 0 |
| HR656S050 | 1 |
| HR656S052 | 0 |
| HR656S053 | 1 |
| HR656S054 | 1 |
| HR656S055 | 1 |
| HR656S056 | 0 |
| HR656S057 | 1 |
| HR656S058 | 0 |
| HR656S059 | 1 |
| HR656S060 | 0 |
| HR656S061 | 0 |
| HR656S062 | 1 |
| HR656S064 | 0 |
| HR656S065 | 1 |
| HR656S066 | 1 |
| HR656S067 | 1 |
| HR656S068 | 0 |
| HR656S069 | 1 |
| HR656S070 | 0 |
| HR656S071 | 1 |
| HR656S072 | 1 |
| HR656S073 | 0 |
| HR656S074 | 1 |
| HR656S075 | 1 |
| HR656S076 | 1 |
| HR656S077 | 1 |
| HR656S078 | 0 |
| HR656S079 | 1 |
| HR656S081 | 1 |
| HR658S001 | 0 |
| HR658S002 | 1 |
| HR658S003 | 1 |
| HR658S004 | 0 |
| HR658S005 | 0 |
| HR658S007 | 0 |
| HR658S010 | 0 |
| HR658S011 | 1 |
| HR658S012 | 1 |
| HR658S013 | 1 |
| HR658S015 | 1 |
| HR658S016 | 0 |
| HR658S018 | 1 |
| HR658S020 | 0 |
| HR658S022 | 0 |
| HR658S024 | 0 |
| HR658S025 | 1 |
| HR658S026 | 1 |
| HR658S027 | 0 |
| HR658S029 | 0 |
| HR658S030 | 1 |
| HR658S031 | 0 |
| HR658S032 | 0 |
| HR658S035 | 1 |
| HR658S036 | 1 |
| HR658S037 | 0 |
| HR658S038 | 1 |
| HR658S039 | 0 |
| HR658S042 | 0 |
| HR658S044 | 1 |
| HR658S045 | 1 |
| HR658S046 | 0 |
| HR658S047 | 1 |
| HR658S048 | 1 |
| HR658S049 | 1 |
| HR658S050 | 1 |
| HR620S001 | 0 |
| HR620S002 | 1 |
| HR620S003 | 1 |
| HR620S004 | 1 |
| HR620S005 | 1 |
| HR620S006 | 1 |
| HR620S007 | 0 |
| HR620S008 | 1 |
| HR620S009 | 0 |
| HR620S010 | 0 |
| HR620S011 | 1 |
| HR620S012 | 1 |
| HR620S013 | 0 |
| HR620S015 | 1 |
| HR620S016 | 0 |
| HR620S017 | 0 |
| HR620S018 | 1 |
| HR620S020 | 0 |
| HR620S021 | 0 |
| HR620S022 | 0 |
| HR620S023 | 0 |
| HR620S024 | 1 |
| HR620S025 | 1 |
| HR620S026 | 1 |
| HR620S028 | 0 |
| HR620S029 | 1 |
| HR620S031 | 0 |
| HR620S033 | 0 |
| HR620S034 | 0 |
| HR620S035 | 0 |
| HR620S038 | 1 |
| HR620S039 | 0 |
| HR620S040 | 0 |
| HR620S041 | 1 |
| HR620S042 | 0 |
| HR620S043 | 1 |
| HR620S044 | 1 |
| HR620S045 | 1 |
| HR620S046 | 0 |
| HR620S048 | 0 |
| HR620S049 | 0 |
| HR620S050 | 0 |
| HR620S051 | 1 |
| HR620S052 | 1 |
| HR620S053 | 0 |
| HR620S054 | 0 |
| HR620S055 | 1 |
| HR620S056 | 1 |
| HR620S057 | 1 |
| HR620S058 | 0 |
| HR620S060 | 0 |
| HR620S061 | 0 |
| HR620S062 | 0 |
| HR620S064 | 0 |
| HR620S065 | 1 |
| HR620S066 | 1 |
| HR620S067 | 0 |
| HR620S068 | 0 |
| HR620S069 | 0 |
| HR620S070 | 0 |
| HR620S071 | 0 |
| HR620S073 | 1 |
| HR620S074 | 1 |
| HR620S075 | 0 |
| HR620S076 | 1 |
| HR620S078 | 0 |
| HR630S050* | 1 |
| HR630S053* | 1 |
| HR630S002* | 0 |
| HR630S014* | 0 |
| HR623S014* | 0 |
| HR623S013* | 1 |
| HR623S024* | 1 |
| HR623S050* | 1 |
| HR655S034* | 1 |
| HR655S019* | 1 |
| HR655S015* | 0 |
| HR624S017* | 1 |

1—HR; responds like Rasmusson
0—No response or low chlorosis; responds like other parent
*RIL chosen for recombination event in the GWAS interval

Table 4. Responses of Wheat Varieties to P. syringae DC3000(D36E) Expressing AvrPphB.

The second leaves of 14-day old wheat seedlings were inoculated with P. syringae DC3000(D36E) carrying AvrPphB (OD₆₀₀=0.5) by infiltration with a needleless syringe. Wheat responses were scored as no response, low chlorosis, or chlorosis three days post-inoculation. Wheat varieties were obtained from the U.S. Department of Agriculture Wheat Germplasm Collection or generously provided by Scot Hulbert (Washington State University).

TABLE 4
Responses of wheat varieties to P. syringae
DC3000(D36E) expressing AvhrPphB.

| Wheact Line | Response |
|---|---|
| Thatcher (Cltr10003) | Chlorosis |
| Penawawa (PI495916) | Chlorosis |
| Neepawa (Cltr15073) | Chlorosis |
| Fielder (Cltr17268) | Chlorosis |
| Avocet S (Hulbert) | Chlorosis |
| Bliss (Cltr486350) | Chlorosis |
| Colano (Cltr15333) | Chlorosis |
| Lew (Cltr17429) | Chlorosis |
| Scarlet (Hulbert) | Chlorosis |
| Zak (Hulbert) | Chlorosis |
| Nabob (Cltr8869) | Chlorosis |
| Thorne (Cltr11856) | Chlorosis |
| Sterling (Cltr17859) | Chlorosis |
| Wichita (Cltr11952) | Chlorosis |
| Blanca (PI501533) | Chlorosis |
| Saunders (Cltr12567) | Chlorosis |
| Chinook (Cltr13220) | Chlorosis |
| Chaparral (Cltr14076) | Chlorosis |
| Crim (Cltr13465) | Chlorosis |
| Rushmore (Cltr12273) | Chlorosis |
| Sunstar Promise (PI585232) | Chlorosis |
| Newthatch (Cltr12318) | Chlorosis |
| Vandal (PI546056) | Low chlorosis |
| Ace (Cltr13384) | Low chlorosis |
| USU-Apogee (PI592742) | Low chlorosis |
| Justin (Cltr13462) | Low chlorosis |
| Polk (Cltr13773) | Low chlorosis |
| Cadet (Cltr12053) | Low chlorosis |
| Wawawai (PI574538) | Low chlorosis |
| Centana (Cltr12974) | No response |
| Eltan (Hulbert) | No response |
| Jefferson (PI603040) | No response |
| WA6101 (Cltr17690) | No response |
| Alpowa (PI566596) | No response |

TABLE 5
Amino Acid Conservative Substitutions.

| | Side | | | Preferred |
|---|---|---|---|---|
| | Chain | Side Chain | Hydropathy | Conservative |
| Residue | Polarity | pH | Index | Substitution |
| Ala (A) | Non-polar | Neutral | 1.8 | Ser |
| Arg (R) | Polar | Basic (strongly) | −4.5 | Lys, Gln |
| Asn (N) | Polar | Neutral | −3.5 | Gln, His |
| Asp (D) | Polar | Acidic | −3.5 | Glu |
| Cys (C) | Non-polar | Neutral | 2.5 | Ser |
| Gln (Q) | Polar | Neutral | −3.5 | Asn, Lys |
| Glu (E) | Polar | Acidic | −3.5 | Asp |
| Gly (G) | Non-polar | Neutral | −0.4 | Pro |
| His (H) | Polar | Basic (weakly) | −3.2 | Asn, Gln |
| Ile (I) | Non-polar | Neutral | 4.5 | Leu, Val |
| Leu (L) | Non-polar | Neutral | 3.8 | Ile, Val |
| Lys (K) | Polar | Basic | −3.9 | Arg, Gln |
| Met (M) | Non-polar | Neutral | 1.9 | Leu, Ile |
| Phe (F) | Non-polar | Neutral | 2.8 | Met, Leu, Tyr |
| Pro (P) | Non-polar | Neutral | −1.6 | Gly |
| Ser (S) | Polar | Neutral | −0.8 | Thr |
| Thr (T) | Polar | Neutral | −0.7 | Ser |
| Trp (W) | Non-polar | Neutral | −0.9 | Tyr |
| Tyr (Y) | Polar | Neutral | −1.3 | Trp, Phe |
| Val (V) | Non-polar | Neutral | 4.2 | Ile, Leu |

### PUBLICATIONS CITED

These publications are incorporated by reference to the extent they relate materials and methods disclosed herein.
Ade, J, DeYoung, B J, Golstein, C, and Innes, R W. (2007). Indirect activation of a plant nucleotide binding site-leucine-rich repeat protein by a bacterial protease. Proc Natl Acad Sci USA 104, 2531-2536.
Aoyama, T., and Chua, N. H. 1997. A glucocorticoid-mediated transcriptional induction system in transgenic plants. Plant J. 11:605-612.
Caldwell, K S, and Michelmore, R W. (2009). Arabidopsis thaliana genes encoding defense signaling and recognition proteins exhibit contrasting evolutionary dynamics. Genetics 181, 671-684.
Carter, M E, Helm, M, Chapman, A V E, Wan, E, Restrepo Sierra, A M, Innes, R W, Bogdanove, A J, and Wise, R P. (2019). Convergent evolution of effector protease recognition by Arabidopsis and barley. Mol Plant Microbe Interact, DOI: 10.1094/MPMI-07-18-0202-FI
Cutler, S. R., Ehrhardt, D. W., Griffitts, J. S., and Somerville, C. R. (2000). Random GFP::cDNA fusions enable visualization of subcellular structures in cells of Arabidopsis at a high frequency. Proc. Natl. Acad. Sci. USA 97:3718-3723.
DeYoung, B. J., Qi, D., Kim, S.-H., Burke, T. P., and Innes, R. W. (2012). Activation of a plant nucleotide binding-leucine rich repeat disease resistance protein by a modified self protein. Cell. Microbiol. 14:1071-1084
Helm, M, Qi, M, Sarkar, S, Yu, H, Whitham, S A, and Innes, R W. (2019). Engineering a decoy substrate in soybean to enable recognition of the Soybean mosaic virus NIa protease. Mol Plant Microbe Interact. DOI: 10.1094/MPMI-12-18-0324-R
Kim, S H, Qi, D, Ashfield, T, Helm, M, and Innes, R W. (2016). Using decoys to expand the recognition specificity of a plant disease resistance protein. Science 351, 684-687.
Kumar, S., Stecher, G., and Tamura, K. (2016). MEGA7: Molecular Evolutionary Genetics Analysis version 7.0 for bigger datasets. Mol. Biol. Evol. 33:1870-1874.
Liu, Z., et al. (2012). The cysteine rich necrotrophic effector SnTox1 produced by Stagonospora nodorum triggers susceptibility of wheat lines harboring Snn1. PLoS Path. 8:e1002467.
Marchler-Bauer, A., and Bryant, S. H. (2004). C D-Search: protein domain annotations on the fly. Nucleic Acids Res. 32:W327-331.
Mascher, M., et al. (2017). A chromosome conformation capture ordered sequence of the barley genome. Nature 544:427-433.
Mengiste, T, et. al. (2010). Receptor-like cytoplasmic kinases integrate signaling from multiple plant immune receptors and are targeted by a Pseudomonas syringae effector. Cell Host Microbe 7, 290-301.
Qi, D., DeYoung, B. J., and Innes, R. W. (2012). Structure-Function Analysis of the Coiled-Coil and Leucine-Rich Repeat Domains of the RPS5 Disease Resistance Protein. Plant Physiol. 158:1819-1832.
Ronquist, F. et al., (2012). MrBayes 3.2: Efficient Bayesian Phylogenetic Inference and Model Choice Across a Large Model Space. Syst. Biol. 61:539-542.
Shao, F, Golstein et al., (2003). Cleavage of Arabidopsis PBS1 by a bacterial type III effector. Science 301, 1230-1233.
Sievers, F. et al., (2011). Fast, scalable generation of high-quality protein multiple sequence alignments using Clustal Omega. Mol. Syst. Biol. Online, publication/10.1038/msb.2011.75
Steuernagel, B. et al., (2015). NLR-parser: rapid annotation of plant NLR complements. Bioinformatics 31:1665-1667
Swiderski, M R, and Innes, R W. (2001). The Arabidopsis PBS1 resistance gene encodes a member of a novel protein kinase subfamily Plant J 26, 101-112.
Thordal-Christensen, H. et al., (1997). Subcellular localization of H2O2 in plants. H2O2 accumulation in papillae and hypersensitive response during the barley-powdery mildew interaction. The Plant Journal 11:1187-1194.
Vinatzer, B. A. et al., (2006). The type III effector repertoire of Pseudomonas syringae pv. syringae B728a and its role in survival and disease on host and non-host plants. Mol. Microbiol. 62:26-44.
Wei, H.-L. et al., (2015). Pseudomonas syringae pv. tomato DC3000 Type III Secretion Effector Polymutants Reveal an Interplay between HopAD1 and AvrPtoB. Cell Host Microbe 17:752-762.
Xavier, A. et al., (2015). NAM: association studies in multiple populations. Bioinformatics 31:3862-3864.
Zhang, J, Li, et al., (2010). Receptor-like Cytoplasmic Kinases Integrate Signaling from Multiple Plant Immune Receptors and Are Targeted by a Pseudomonas syringae Effector. Cell Host Microbe 7:290-301.

1. A recombinant nucleic acid molecule comprising a nucleotide sequence that encodes a Glycine max AvrPphB susceptible 1 (GmPBS1) substrate protein and a heterologous pathogen-specific protease recognition sequence.

2. The recombinant nucleic acid molecule of claim 1, wherein the nucleotide sequence encodes the heterologous pathogen-specific protease recognition sequence of SEQ ID NO:2.

3. The recombination nucleic acid molecule of claim 1, wherein the nucleotide sequence comprises one of SEQ ID NO:9, SEQ ID NO:11, and SEQ ID NO:13.

4. A fusion protein comprising a Glycine max AvrPphB susceptible 1 (GmPBS1) substrate protein and a heterologous pathogen-specific protease recognition sequence.

5. The fusion protein of claim 4, wherein the heterologous pathogen-specific protease recognition sequence is SEQ ID NO:2.

6. The fusion protein of claim 4 comprising the amino acid sequence selected from the group consisting of SEQ ID NO:10, SEQ ID NO:12, and SEQ ID NO:14.

7. A vector comprising the recombinant nucleic acid molecule according to claim 1.

8. A transformed plant cell comprising the recombinant nucleic acid molecule according to claim 1.

9. The transformed plant cell of claim 8, wherein the plant cell is from a soybean plant.

10. A transformed plant comprising the recombinant nucleic acid molecule according to claim 1.

11. The transformed plant of claim 10, wherein the plant is a soybean plant.

12. A transgenic seed of the transformed plant according to claim 10.

13. A method of protecting a plant from infection by a plant pathogen that secretes at least one specific protease, the method comprising the step of:
introducing to the plant a nucleotide sequence that encodes a Glycine max AvrPphB susceptible 1 (GmPBS1) substrate protein and a heterologous pathogen-specific protease recognition sequence.

14. The method of claim 13, wherein the nucleotide sequence encodes the heterologous pathogen-specific protease recognition sequence of SEQ ID NO:2.

15. The method of claim 13, wherein the nucleotide sequence comprises one of SEQ ID NO:9, SEQ ID NO:11, and SEQ ID NO:13.


SEQUENCE LISTING (55 sequences)

SEQ ID NO: 1 - protein, 7 aa, Artificial Sequence
  Description of Artificial Sequence Synthetic peptide
GDKSHVS

SEQ ID NO: 2 - protein, 7 aa, Artificial Sequence
  Description of Artificial Sequence Synthetic peptide
ESVSLQS

SEQ ID NO: 3 - DNA, 1350 bp, Glycine max
atgggttgcttttcatgtttcgattcgagttccaaggaggatcacaatctccgtcctcagcaccaacccaatcaacctctcccttctcagatttccagattgccctctggagcagacaagttacggtccagaagtaatggaggttccaaaagagaactgcaacagcctcctcccaccgtccaaattgctgctcaaacatttactttccgtgaacttgccgctgcaactaaaaactttagaccagagtcctttgttggggaaggtggttttggaagggtctacaaaggcaggcttgaaaccactgctcagattgttgctgtcaaacagttagacaaaaatggtcttcagggtaatcgggaattccttgtagaggttctcatgctcagtcttctacatcaccctaaccttgtcaatctcattggatactgtgcggatggggatcaacgcctcctcgtttatgaatttatgcctttgggatcattggaagatcaccttcatgatcttccccctgataaggaaccactagattggaacactagaatgaaaattgctgttggtgctgcaaaaggattagaataccttcacgataaggcaaatcctcctgtcatctacagagacttcaagtcatctaacatattacttgatgaaggataccacccaaaactttctgactttggtcttgcgaagcttggtcctgttggtgacaaatcacatgtttctacccgtgtcatgggaacttatggttactgtgctcctgagtatgctatgactggacagctgactgtgaagtctgatgtatatagttttggggtggtcttcttagagctgattactggccgtaaagcaattgacagcacccagccgcagggagaacagaaccttgtcacatgggcacgtccactttttaatgaccgcaggaagttttcaaagttggctgatcccaggctgcaggggcgatttcccatgcggggtctttaccaggctcttgctgtggcatcaatgtgcattcaagaatcagctgcaacgcgtcctctaattggagatgtggtgacagccctctcttatctggccaaccaggcatatgaccccaatggttatagggggtctagtgatgataaaaggaacagagatgataaaggtggaagaatatccaaaaatgatgaagctgggggatctggacgcagatgggacttggaaggatccgagaaagatgactccccacgagaaactgcaaggattttaaacagagatctagatagagaacgagctgtggctgaagccaagatgtggggagagaacttgagacaaaaaagaaaacaaagtttgcagcagggcagtcttgatgcttaa

SEQ ID NO: 4 - protein, 449 aa, Glycine max
MGCFSCFDSSSKEDHNLRPQHQPNQPLPSQISRLPSGADKLRSRSNGGSKRELQQPPPTVQIAAQTFTFRELAAATKNFRPESFVGEGGFGRVYKGRLETTAQIVAVKQLDKNGLQGNREFLVEVLMLSLLHHPNLVNLIGYCADGDQRLLVYEFMPLGSLEDHLHDLPPDKEPLDWNTRMKIAVGAAKGLEYLHDKANPPVIYRDFKSSNILLDEGYHPKLSDFGLAKLGPVGDKSHVSTRVMGTYGYCAPEYAMTGQLTVKSDVYSFGVVFLELITGRKAIDSTQPQGEQNLVTWARPLFNDRRKFSKLADPRLQGRFPMRGLYQALAVASMCIQESAATRPLIGDVVTALSYLANQAYDPNGYRGSSDDKRNRDDKGGRISKNDEAGGSGRRWDLEGSEKDDSPRETARILNRDLDRERAVAEAKMWGENLRQKRKQSLQQGSLDA

SEQ ID NO: 5 - DNA, 1380 bp, Glycine max
atgggttgtttttcgtgcttcgattcgcgggaggacgagatgctgaatcccaatcctcagcaggaaaatcaccaccatgaacatgaacatgatcatgatcttaagccccctgttccttctcgtatttccagattgccgccctctgcctctggagacaagctacggtcaacaacaagtaatggagagtccaaaagggaattggctgctgctgttcaaattgccgctcaaattttcactttccgtgagcttgcagccgcaacgaaaaacttcatgccccaatcctttttaggggaaggtggttttggaagggtctacaagggcctcctggaaaccaccggtcaggttgtggctgttaaacagttagacagagacggtcttcagggtaatcgggaattcctcgttgaggttctcatgctcagtcttctacaccaccctaatcttgtcaatctcattggatactgtgctgatggtgaccaacgcctcttagtttatgaatttatgccattgggatcattggaagaccatcttcatgatcttcccccggataaggaaccattagattggaacaccaggatgaaaatagctgctggggcagcaaaaggattggaataccttcatgacaaagcaaatcctcctgtcatttatagagacttcaaatcatctaatatattacttgatgaaggttatcatccaaagctttcagactttggtcttgcaaagctcggtccagttggtgacaaatcacacgtttccacccgtgtcatgggaacttatggttactgtgccccagaatatgctatgactggacagctgactgtgaagtctgatgtatatagttttggggtagtcttcttggagctgattactggccgtaaagccattgacagcactcgaccccatggagaacaaaaccttgtcacatgggcacgtccactgttcaatgaccgcaggaagtttccaaagttagcagatccacagctgcagggacggtatcccatgcgaggtctttaccaagctctagctgtggcatcaatgtgcattcaagaacaggctgcagcacgtcctctcattggggatgtggtgacagccctttcgtttctagccaaccaggcatatgaccatagggggggaactggtgacgataaaaggaacagagtattgaaaaatggtgaaggtggaggaggaggatctggaggcagatgggatttggaaggatctgagaaagatgactccccacgtgaaactgcaaggatgttaaacagcaacaacagggatcttgatagagaacgtgccgtggctgaagccaagatgtggggagagaattggagagaaaaaagacgacaaagtgcgcagggcagttttgatggttctaacgcttag

SEQ ID NO: 6 - protein, 459 aa, Glycine max
MGCFSCFDSREDEMLNPNPQQENHHHEHEHDHDLKPPVPSRISRLPPSASGDKLRSTTSNGESKRELAAAVQIAAQIFTFRELAAATKNFMPQSFLGEGGFGRVYKGLLETTGQVVAVKQLDRDGLQGNREFLVEVLMLSLLHHPNLVNLIGYCADGDQRLLVYEFMPLGSLEDHLHDLPPDKEPLDWNTRMKIAAGAAKGLEYLHDKANPPVIYRDFKSSNILLDEGYHPKLSDFGLAKLGPVGDKSHVSTRVMGTYGYCAPEYAMTGQLTVKSDVYSFGVVFLELITGRKAIDSTRPHGEQNLVTWARPLFNDRRKFPKLADPQLQGRYPMRGLYQALAVASMCIQEQAAARPLIGDVVTALSFLANQAYDHRGGTGDDKRNRVLKNGEGGGGGSGGRWDLEGSEKDDSPRETARMLNSNNRDLDRERAVAEAKMWGENWREKRRQSAQGSFDGSNA

SEQ ID NO: 7 - DNA, 1401 bp, Glycine max
atgggttgtttttcgtgcttcgattcgcgggaggacgagaagctgaatcccaatcctcagcaggaaaaccaccagcatgaacatgaacatgaacatgatcttaagccccctgttccttctcgtatatccagattgccgccctctgcctctgcctctgcctctgcctctgcagtaggagcagacaagctacgctcaacaacaagtaatggtaatggagagtccactgctgttcaaattgcggctcaaacattcagtttccgtgagcttgcagccgcaacgaaaaacttcaggccccaatcctttttgggtgaaggtggttttggaagggtctacaagggccgcctggaaaccaccggtcaggttgtggctgttaaacagttagacagaaacggtcttcagggtaatcgggaattcctcgtcgaggttctcatgctcagtcttctacaccaccctaatcttgtcaatctcattggatactgtgccgatggggaccaacgcctcttagtttatgaatttatgccttttggatctttggaggaccaccttcatgatcttcccccggacaaggaaccattagattggaacaccaggatgaaaatagctgctggggcagcaaaaggattggaataccttcacgacaaagcaaaccctcctgtcatttatagagacttcaaatcatctaatatattacttgatgaaggttatcatccaaagctttcagactttggtcttgcaaagcttggtccagttggtgacaaatcacacgtttcaacccgtgtcatgggaacttatggttactgtgccccagaatatgctatgactggacagctgactgtgaagtctgatgtatatagttttggggtagtcttcttggagctgattactggccgtaaagccattgacagcactcgaccccatggagaacaaaaccttgtcacatgggcacgtccactgttcagtgaccgcaggaagtttccaaagttagcagatccacagctgcagggacggtatcccatgcggggtctttaccaagctctagctgtggcatcaatgtgcattcaagaacaggctgcagcacgtccactcattggggatgtggtgacagccctttcttttctggccaaccaggcatatgaccataggggagctggtgatgataaaaagaacagggatgataaaggtggaagaatattgaaaaatgatgtaggtggaggatctggacgcagatgggatttggaaggatctgagaaagatgactccccacgtgaaactgcaaggatgttaaacaacagggatcttgatagagaacgtgccgtggctgaagccaagatatggggagagaattggagagaaaaaagacgacaaagtgcgcagggcagttttgatggttctaacgcttag

SEQ ID NO: 8 - protein, 465 aa, Glycine max
MGCFSCFDSREDEKLNPNPQQENHQHEHEHEHDLKPPVPSRISRLPPSASASASASVGADKLRSTTSNGNGESTAVQIAAQTFSFRELAAATKNFRPQSFLGEGGFGRVYKGRLETTGQVVAVKQLDRNGLQGNREFLVEVLMLSLLHHPNLVNLIGYCADGDQRLLVYEFMPFGSLEDHLHDLPPDKEPLDWNTRMKIAAGAAKGLEYLHDKANPPVIYRDFKSSNILLDEGYHPKLSDFGLAKLGPVGDKSHVSTRVMGTYGYCAPEYAMTGQLTVKSDVYSFGVVFLELITGRKAIDSTRPHGEQNLVTWARPLFSDRRKFPKLADPQLQGRYPMRGLYQALAVASMCIQEQAAARPLIGDVVTALSFLANQAYDHRGAGDDKKNRDDKGGRILKNDVGGGSGRRWDLEGSEKDDSPRETARMLNNRDLDRERAVAEAKIWGENWREKRRQSAQGSFDGSNA

SEQ ID NO: 9 - DNA, 1350 bp, Artificial Sequence
  Description of Artificial Sequence Synthetic polynucleotide
atgggttgcttttcatgtttcgattcgagttccaaggaggatcacaatctccgtcctcagcaccaacccaatcaacctctcccttctcagatttccagattgccctctggagcagacaagttacggtccagaagtaatggaggttccaaaagagaactgcaacagcctcctcccaccgtccaaattgctgctcaaacatttactttccgtgaacttgccgctgcaactaaaaactttagaccagagtcctttgttggggaaggtggttttggaagggtctacaaaggcaggcttgaaaccactgctcagattgttgctgtcaaacagttagacaaaaatggtcttcagggtaatcgggaattccttgtagaggttctcatgctcagtcttctacatcaccctaaccttgtcaatctcattggatactgtgcggatggggatcaacgcctcctcgtttatgaatttatgcctttgggatcattggaagatcaccttcatgatcttccccctgataaggaaccactagattggaacactagaatgaaaattgctgttggtgctgcaaaaggattagaataccttcacgataaggcaaatcctcctgtcatctacagagacttcaagtcatctaacatattacttgatgaaggataccacccaaaactttctgactttggtcttgcgaagcttggtcctgttgaaagcgtatcattacagtctacccgtgtcatgggaacttatggttactgtgctcctgagtatgctatgactggacagctgactgtgaagtctgatgtatatagttttggggtggtcttcttagagctgattactggccgtaaagcaattgacagcacccagccgcagggagaacagaaccttgtcacatgggcacgtccactttttaatgaccgcaggaagttttcaaagttggctgatcccaggctgcaggggcgatttcccatgcggggtctttaccaggctcttgctgtggcatcaatgtgcattcaagaatcagctgcaacgcgtcctctaattggagatgtggtgacagccctctcttatctggccaaccaggcatatgaccccaatggttatagggggtctagtgatgataaaaggaacagagatgataaaggtggaagaatatccaaaaatgatgaagctgggggatctggacgcagatgggacttggaaggatccgagaaagatgactccccacgagaaactgcaaggattttaaacagagatctagatagagaacgagctgtggctgaagccaagatgtggggagagaacttgagacaaaaaagaaaacaaagtttgcagcagggcagtcttgatgcttaa

SEQ ID NO: 10 - protein, 449 aa, Artificial Sequence
  Description of Artificial Sequence Synthetic polypeptide
MGCFSCFDSSSKEDHNLRPQHQPNQPLPSQISRLPSGADKLRSRSNGGSKRELQQPPPTVQIAAQTFTFRELAAATKNFRPESFVGEGGFGRVYKGRLETTAQIVAVKQLDKNGLQGNREFLVEVLMLSLLHHPNLVNLIGYCADGDQRLLVYEFMPLGSLEDHLHDLPPDKEPLDWNTRMKIAVGAAKGLEYLHDKANPPVIYRDFKSSNILLDEGYHPKLSDFGLAKLGPVESVSLQSTRVMGTYGYCAPEYAMTGQLTVKSDVYSFGVVFLELITGRKAIDSTQPQGEQNLVTWARPLFNDRRKFSKLADPRLQGRFPMRGLYQALAVASMCIQESAATRPLIGDVVTALSYLANQAYDPNGYRGSSDDKRNRDDKGGRISKNDEAGGSGRRWDLEGSEKDDSPRETARILNRDLDRERAVAEAKMWGENLRQKRKQSLQQGSLDA

SEQ ID NO: 11 - DNA, 1380 bp, Artificial Sequence
  Description of Artificial Sequence Synthetic polynucleotide
atgggttgtttttcgtgcttcgattcgcgggaggacgagatgctgaatcccaatcctcagcaggaaaatcaccaccatgaacatgaacatgatcatgatcttaagccccctgttccttctcgtatttccagattgccgccctctgcctctggagacaagctacggtcaacaacaagtaatggagagtccaaaagggaattggctgctgctgttcaaattgccgctcaaattttcactttccgtgagcttgcagccgcaacgaaaaacttcatgccccaatcctttttaggggaaggtggttttggaagggtctacaagggcctcctggaaaccaccggtcaggttgtggctgttaaacagttagacagagacggtcttcagggtaatcgggaattcctcgttgaggttctcatgctcagtcttctacaccaccctaatcttgtcaatctcattggatactgtgctgatggtgaccaacgcctcttagtttatgaatttatgccattgggatcattggaagaccatcttcatgatcttcccccggataaggaaccattagattggaacaccaggatgaaaatagctgctggggcagcaaaaggattggaataccttcatgacaaagcaaatcctcctgtcatttatagagacttcaaatcatctaatatattacttgatgaaggttatcatccaaagctttcagactttggtcttgcaaagctcggtccagttgaaagcgtatcattacagtctacccgtgtcatgggaacttatggttactgtgccccagaatatgctatgactggacagctgactgtgaagtctgatgtatatagttttggggtagtcttcttggagctgattactggccgtaaagccattgacagcactcgaccccatggagaacaaaaccttgtcacatgggcacgtccactgttcaatgaccgcaggaagtttccaaagttagcagatccacagctgcagggacggtatcccatgcgaggtctttaccaagctctagctgtggcatcaatgtgcattcaagaacaggctgcagcacgtcctctcattggggatgtggtgacagccctttcgtttctagccaaccaggcatatgaccatagggggggaactggtgacgataaaaggaacagagtattgaaaaatggtgaaggtggaggaggaggatctggaggcagatgggatttggaaggatctgagaaagatgactccccacgtgaaactgcaaggatgttaaacagcaacaacagggatcttgatagagaacgtgccgtggctgaagccaagatgtggggagagaattggagagaaaaaagacgacaaagtgcgcagggcagttttgatggttctaacgcttag

SEQ ID NO: 12 - protein, 459 aa, Artificial Sequence
  Description of Artificial Sequence Synthetic polypeptide
MGCFSCFDSREDEMLNPNPQQENHHHEHEHDHDLKPPVPSRISRLPPSASGDKLRSTTSNGESKRELAAAVQIAAQIFTFRELAAATKNFMPQSFLGEGGFGRVYKGLLETTGQVVAVKQLDRDGLQGNREFLVEVLMLSLLHHPNLVNLIGYCADGDQRLLVYEFMPLGSLEDHLHDLPPDKEPLDWNTRMKIAAGAAKGLEYLHDKANPPVIYRDFKSSNILLDEGYHPKLSDFGLAKLGPVESVSLQSTRVMGTYGYCAPEYAMTGQLTVKSDVYSFGVVFLELITGRKAIDSTRPHGEQNLVTWARPLFNDRRKFPKLADPQLQGRYPMRGLYQALAVASMCIQEQAAARPLIGDVVTALSFLANQAYDHRGGTGDDKRNRVLKNGEGGGGGSGGRWDLEGSEKDDSPRETARMLNSNNRDLDRERAVAEAKMWGENWREKRRQSAQGSFDGSNA

SEQ ID NO: 13 - DNA, 1398 bp, Artificial Sequence
  Description of Artificial Sequence Synthetic polynucleotide
atgggttgtttttcgtgcttcgattcgcgggaggacgagaagctgaatcccaatcctcagcaggaaaaccaccagcatgaacatgaacatgaacatgatcttaagccccctgttccttctcgtatatccagattgccgccctctgcctctgcctctgcctctgcctctgtaggagcagacaagctacgctcaacaacaagtaatggtaatggagagtccactgctgttcaaattgcggctcaaacattcagtttccgtgagcttgcagccgcaacgaaaaacttcaggccccaatcctttttgggtgaaggtggttttggaagggtctacaagggccgcctggaaaccaccggtcaggttgtggctgttaaacagttagacagaaacggtcttcagggtaatcgggaattcctcgtcgaggttctcatgctcagtcttctacaccaccctaatcttgtcaatctcattggatactgtgccgatggggaccaacgcctcttagtttatgaatttatgccttttggatctttggaggaccaccttcatgatcttcccccggacaaggaaccattagattggaacaccaggatgaaaatagctgctggggcagcaaaaggattggaataccttcacgacaaagcaaaccctcctgtcatttatagagacttcaaatcatctaatatattacttgatgaaggttatcatccaaagctttcagactttggtcttgcaaagcttggtccagttgaaagcgtatcattacagtctacccgtgtcatgggaacttatggttactgtgccccagaatatgctatgactggacagctgactgtgaagtctgatgtatatagttttggggtagtcttcttggagctgattactggccgtaaagccattgacagcactcgaccccatggagaacaaaaccttgtcacatgggcacgtccactgttcagtgaccgcaggaagtttccaaagttagcagatccacagctgcagggacggtatcccatgcggggtctttaccaagctctagctgtggcatcaatgtgcattcaagaacaggctgcagcacgtccactcattggggatgtggtgacagccctttcttttctggccaaccaggcatatgaccataggggagctggtgatgataaaaagaacagggatgataaaggtggaagaatattgaaaaatgatgtaggtggaggatctggacgcagatgggatttggaaggatctgagaaagatgactccccacgtgaaactgcaaggatgttaaacaacagggatcttgatagagaacgtgccgtggctgaagccaagatatggggagagaattggagagaaaaaagacgacaaagtgcgcagggcagttttgatggttctaacgcttag

SEQ ID NO: 14 - protein, 465 aa, Artificial Sequence
  Description of Artificial Sequence Synthetic polypeptide
MGCFSCFDSREDEKLNPNPQQENHQHEHEHEHDLKPPVPSRISRLPPSASASASASVGADKLRSTTSNGNGESTAVQIAAQTFSFRELAAATKNFRPQSFLGEGGFGRVYKGRLETTGQVVAVKQLDRNGLQGNREFLVEVLMLSLLHHPNLVNLIGYCADGDQRLLVYEFMPFGSLEDHLHDLPPDKEPLDWNTRMKIAAGAAKGLEYLHDKANPPVIYRDFKSSNILLDEGYHPKLSDFGLAKLGPVESVSLQSTRVMGTYGYCAPEYAMTGQLTVKSDVYSFGVVFLELITGRKAIDSTRPHGEQNLVTWARPLFSDRRKFPKLADPQLQGRYPMRGLYQALAVASMCIQEQAAARPLIGDVVTALSFLANQAYDHRGAGDDKKNRDDKGGRILKNDVGGGSGRRWDLEGSEKDDSPRETARMLNNRDLDRERAVAEAKIWGENWREKRRQSAQGSFDGSNA

SEQ ID NO: 15 - DNA, 702 bp, Soybean mosaic virus
atgagcaagtctgtctacaaaggacttagagattatagtggcatttccacattaatatgtcaactcacaaattcatcagatgggcataaagaaacaatgtttggggtaggctatggttctttcattattacaaatggacacttgttcagaaggaacaatggaatgctcacagttaagacatggcatggcgagtttgtaatacacaacacaacacagctcaagatacattttattcaagggaaggatgtgattctgattcgcatgccaaaggattttcctccattcggaaaacgcaacctctttagacaaccaaagcgtgaggaacgagtttgtatggttgggacaaacttccaagagaagagcttgcgtgcaacagtttcagaatcttctatgatattgcctgaggggaagggttctttctggatacattggataacaacccaggatggtttttgtgggttacctcttgtttctgttaatgatgggcacattgttggaatacatggattaacatccaatgattcagaaaagaacttctttgttccactcactgatgggtttgagaaagaatacctagagaatgctgataacttgtcatgggacaagcattggttttgggaaccaagtaagatagcatggggctctttgaacttagttgaggaacaaccaaaagaggagttcaaaatatcaaagcttgtgtca

SEQ ID NO: 16 - protein, 456 aa, Arabidopsis thaliana
MGCFSCFDSSDDEKLNPVDESNHGQKKQSQPTVSNNISGLPSGGEKLSSKTNGGSKRELLLPRDGLGQIAAHTFAFRELAAATMNFHPDTFLGEGGFGRVYKGRLDSTGQVVAVKQLDRNGLQGNREFLVEVLMLSLLHHPNLVNLIGYCADGDQRLLVYEFMPLGSLEDHLHDLPPDKEALDWNMRMKIAAGAAKGLEFLHDKANPPVIYRDFKSSNILLDEGFHPKLSDFGLAKLGPTGDKSHVSTRVMGTYGYCAPEYAMTGQLTVKSDVYSFGVVFLELITGRKAIDSEMPHGEQNLVAWARPLFNDRRKFIKLADPRLKGRFPTRALYQALAVASMCIQEQAATRPLIADVVTALSYLANQAYDPSKDDSRRNRDERGARLITRNDDGGGSGSKFDLEGSEKEDSPRETARILNRDINRERAVAEAKMWGESLREKRRQSEQGTSESNSTG

SEQ ID NO: 17 - protein, 30 aa, Arabidopsis thaliana
DFGLAKLGPTGDKSHVSTRVMGTYGYCAPE

SEQ ID NO: 18 - protein, 30 aa, Hordeum vulgare
DFGLAKLGPVGDKSHVSTRVMGTYGYCAPE

SEQ ID NO: 19 - protein, 30 aa, Hordeum vulgare
DFGLAKLGPVGDKTHVTTRVMGTYGYCAPE

SEQ ID NO: 20 - protein, 937 aa, Triticum aestivum
MVSALAGVMTSVIAKLTALLGEEYAKLKGVHREVEFMKDELSSMNALLQRLAEVDRDLDVQTKEWRDQVREMSYDIEDCIDDFMKSLGQTDSAKAAGLVQSVLQQLKALRARHQISSQIQGLKARVEDASKRRMRYKLDERTFEPSISRAIDPRLPSLYAEPDGLVGIDKPRSELIECLMEGMGASVQQQKVISIVGPGGLGKTTLANEVFRKLEGQFQCRAFVSLSQQPDVSKILRNILSQVCQQELPSTDIQDEGKLIDTIREVLKNKRYLVVIDDIWSTQAWKIIKCSLFLNDLGSRIMTTTRSIDIAKSCCSRRHDRVYEIMPLTAANSKSLFFKRIFGSEDICPPQLEEVSSEILKKCGGSPLAILTIASLLANKDCTNEEWEWVYNSIGSTLGKDPGVEEMRRILSLSYDDLPHHLKTCLLYLSIFPEDYEIERDRLVRRWIAEGFIDTNGGRDLEEIGERYFNDLINRSMLQPAEIQYDGQVVSCRVHDMILDLLTSKSMEENFATFFRNQNEILVLQHKIRRLSLSYYDQEHIMLPSTAIISHCRSLSIVGYAEKMPSLSKFRVLRVLDIENGEEMESNCFEHLRKLFQLKYLRLHVRSISALPEQLGELQHLKTLDMGWTKITKMPKSIVQLQHLTCLRVSNLELPEGIGNLQALQELSDIKVNRYSMASCLLELGSLTKLKILGLRWYIVSTHSNKDTFVDNLVSSLRKLGRFSLRSICIRSYHGYSMEFLLDSWFPSPYLMQKFQMGTYYNFPRVPPWIVSLDKLTYLDINIDPVDEETLEILGELPALLFLWLMSKSAAPKQRLIISSSMFVCLREFHFTCWSNGEGLMFEAGAMPRLEKLWVPFDAGSGLDFGIQHLSSLRHLAVEIICVGATARDVEALEEAIRDAAHLLPNRPAVEFRTWDDEKMAGEEGQGVTEEEIHASG

SEQ ID NO: 21 - protein, 939 aa, Hordeum vulgare
MVSALAGVMTSVIGKLTALLGEEYAKLKGVHREVEFMKDELSSMNALLQRLAEADRDLDVQTKEWRDQVREMSYDIEDCMDDFMKSLGQTDSAQTAGLVQSVVQQLKALRARHQISSKIQGLKARVEDASKRRMRYKLDERTFEPSISRAINPRLPSLYAEPDGLVGIDKPRDELIKCLMEGMGASVQQQKVLSIVGPGGLGKTTLANEVYRKLEGQFQCRAFVSLSQQPDVNKILRNILSQVCQQELPSTSVQDEGKLIDAIREVLKNKRYLVVIDDIWSTQAWKIIKCSLFLNDLGSRIMTTTRSIDIAKSCCSRRHDRVYEIMPLTTANSKGLFFKRIFGSEDICPPQLEEISSEILKKCGGSPLAILTIASLLANKDSTNEEWKWVYNSIGSTLGKDPGVEEMRRILSLSYDDLPHHLKTCLLYLSIFPEDYEIERDRLIRRWIAEGFIDTDGGRDLEEIGECYFNDLINRSMLEPVKIQYDGQVVSCRVHDMILDLLASKSIEENFATFSGNQNEILVLRHKIRRLSLNYYAQEHTMLPSTAIISHCRSLSIVGYAEKMPSLSKFRVLRVLDIENGEEMESNCFEHLRTLFQLRYLRLHVRSISALPEQLGELQHLRTLDMGWTKITKMPKSIVQLQHLTCLRVSNLELPEGIGNLQALQELSDIKVNRHSTASCLLELGSLTKLKILGLRWSIVSTHGNEDTFVDNLVSSLRKLGRSSLRSICIRSYHGYTMEFLLDSWFPSPHLMQKFQMGTYYNFPRIPPWIASLDKLTYLDINIDPVEEEALEILGELPSLLFLWLTSKSAAPKQRLVVSSSMFVRLKELHFTCWSNGQGLMFEAGAMPRLEKLWVPFDAGSGLDSGIQHLSSLTHLAVEIICVGATARDVEALEEAIRGAARLLPNRPAVEFRTWDDEKMVVEEEEGQGVPEEEIHASG

SEQ ID NO: 22 - protein, 456 aa, Arabidopsis thaliana; identical to SEQ ID NO: 16

SEQ ID NO: 23 - protein, 479 aa, Hordeum vulgare
MGCFPCFDSSSDGELLYPKQGGGGGGGGGNGTGGRTAAAASSSGVGAREERPMVPPRVEKLPAGAEKARAKGNAGMKELSDLRDANGNVLSAQTFTFRQLTAATRNFREECFIGEGGFGRVYKGRLDGGQVVAIKQLNRDGNQGNKEFLVEVLMLSLLHHQNLVNLVGYCADGEQRLLVYEYMPLGSLEDHLHDLPPDKEPLDWNTRMKIAAGAAKGLEYLHDKAQPPVIYRDFKSSNILLGDDFHPKLSDFGLAKLGPVGDKSHVSTRVMGTYGYCAPEYAMTGQLTVKSDVYSFGVVLLELITGRKAIDSTRPHGEQNLVSWARPLFNDRRKLPKMADPGLQGRYPMRGLYQALAVASMCIQSEAASRPLIADVVTALSYLASQIYDPNAIHASKKAGGDQRSRVSDSGRALLKNDEAGSSGHKSDRDDSPREPPPGILNDRERMVAEAKMWGANLREKTRAAANAQGSLDSPTETG

SEQ ID NO: 24 - protein, 464 aa, Hordeum vulgare
MGFLSCLFRCPEEEEVVVKEHDDNEDSSGIDHGVASESSESVPLRAESTHIEGIQRNGTNNEATIFTLRELVDATKNFSQDSQLGRGGFGCVYKAYLNDGQVVAVKQLDLNGLQGNREFLVEVLMLNLLHHPNLVNLIGYCVDGDQRLLVYEYMPLGSLEDHLHDLPPNKEPLDWTTRMKIAAGAAAGLEYLHDKANPPVIYRDIKPSNILLAEGYHAKLSDFGLAKLGPVGDKTHVTTRVMGTYGYCAPEYAATGQLTNKSDIYSFGVVFLELITGRRALDSNRPREEQDLVSWARPLFKDQRKFPKMADPLLRGRFPKRGLYQALAIAAMCLQEKSRNRPLIREVAAALSYLSSQTYNGNDAAGRRCLDGPSTPKVSEEQVNQDDALPSQLGAQTSMHDRMNDFIPEGKEHCRSGSNRGVRGRVVPNGVDRDRALADANVWAEAWRRHEKASKVRVTDEILG

SEQ ID NO: 25 - DNA, 1371 bp, Arabidopsis thaliana
atgggttgtttctcgtgttttgattcgagtgatgacgagaagctgaatccagttgatgaatctaatcatggtcagaagaaacaatcacaaccgacagtatccaataacatatctggactcccttcaggtggggagaagcttagctcaaagaccaatggaggatcaaaaagggagctactgcttccaagggatggacttggacaaattgctgctcatacatttgctttccgcgagcttgctgctgcaactatgaactttcatcctgacactttcttaggcgaaggtggatttggacgtgtctacaaaggaaggcttgacagcaccggtcaggttgttgctgttaaacaactagacaggaatggtctacaaggtaacagagaatttctggtagaggttcttatgctcagtcttcttcatcatcccaacttagtcaaccttattggttattgtgctgatggagatcaacgcctcttggtctacgagtttatgccgttaggatcattggaagatcacctccacgatcttccaccggataaggaggccttagattggaacatgagaatgaaaatagctgctggtgcggcgaaaggattggaatttctacatgataaggcaaaccctccggttatttatagagattttaagtcatcaaatattttactggatgagggtttccaccctaagctttctgattttggacttgctaaactcggaccaacgggagacaaatctcatgtctccactagagttatgggaacttatggttattgtgctcccgagtacgcaatgacgggacaattgacagtaaaatcagatgtctacagttttggtgtggtttttctcgagctcattactggtcgcaaagctatagacagcgagatgcctcatggagagcagaacctggtggcttgggctcgcccattgttcaacgacaggcgaaagttcataaaactggctgatccaaggttaaaggggcggtttccaacgcgtgcactctaccaagctttagctgtggcatcaatgtgcatccaagaacaggcggctacacgtcctctcatagcagatgttgtcactgcactctcctatcttgcaaaccaagcttatgatccaagtaaagatgatagtagaagaaaccgggatgaaagaggtgcaaggttaataacaaggaacgacgatggaggtggctcgggaagtaaattcgatttagaaggttcagagaaagaagattcaccgagagagacagctcggatattgaaccgagatatcaatagggagcgtgcggttgcagaggctaagatgtggggagagagtttgagggagaaacgaagacagagcgagcagggtacttcagagagcaacagtaccgggtag

SEQ ID NO: 26 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaatccaggggctcaaggcacgtgttgaagatgcaagcaagcgacgtatgaggtataagcttgatgagcgcaccttcgagcctagcatctcaagggctatcgaccctcgtttgccttcactctatgctgagcctgatgggcttgtcggtattgacaagccaagagacgagctcattaagtgcctaatggaggggatgggtgcatcagtgcagcagcaaaaggtgttatctattgtgggtcctgggggtctcggtaaaactacacttgccaatgaggtgtaccgtaaactggaaggccagttccagtgtcgagcttttgtttccttgtcacaacaaccagatgtgaataagatcttaagaaatatactctctcaagtctgccagcaagagcttcctagcacaagtgtacaggacgagggaaaactcattgacgcaatcagagaagttttaaagaacaagaggtacattcaccaactttgaattagttggctgttggtactgcatcattttttcttcagcattgatgcttatttagtagcctagccattctactatagtctactgttgtttaattaaattaaatatagatgattttgtttcattttaagcatatatatgcaattgtgaatagtgctttgattcacagttcatgaggttatctatctaaagggctcaaaagacttcctttaaatatcgtaaccccctatgataaattcaactttttgtggtgtggccaaaccattggggaaggggataatggctgtctgtaatagtaatatatagaagagaaatgattctgtccccataaacagtgaatttcaagaaacctgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgacaacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgccctagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgctggagctgggcagtctgaccaaactgaaaatccttgggctacgctggtcaattgtcagtacacacggtaacgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacgtactacaacttccccagagttcctccttggatcgcgtcgctggacaagctcacatacctagatatcaatatcgatccagtagaagaggaagcactggagatccttggagaattgccttctttgctgtttctctggctgacatcgaaatcggctgctcccaaacagcggctcgtcgtaagcagcagcatgtttgtgcgtctgaaggagctccacttcacctgctggagcaatgggcaaggactgatgtttgaggccggggccatgccgaggctcgagaagctgtgggttccgtttgacgcaggcagcggtcttgattttggcatccagcacctctcttccctcacgcatcttgccgtcgagatcatttgcgtcggcgcgaccgctcgggacgtagaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 27 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaatccaggggctcaaggcacgtgttgaagatgcaagcaagcgacgtatgaggtataagcttgatgagcgcaccttcgagcctagcatctcaagggctatcgaccctcgtttgccttcactctatgctgagcctgatgggcttgtcggtattgacaagccaagagacgagctcattaagtgcctaatggaggggatgggtgcatcagtgcagcagcaaaaggtgttatctattgtgggtcctgggggtctcggtaaaactacacttgccaatgaggtgtaccgtaaactggaaggccagttccagtgtcgagcttttgtttccttgtcacaacaaccagatgtgaataagatcttaagaaatatactctctcaagtctgccagcaagagcttcctagcacaagtgtacaggacgagggaaaactcattgacgcaatcagagaagttttaaagaacaagaggtacattcaccaactttgaattagttggctgttggtactgcatcattttttcttcagcattgatgcttatttagtagcctagccattctactatagtctactgttgtttaattaaattaaatatagatgattttgtttcattttaagcatatatatgcaattgtgaatagtgctttgattcacagttcatgaggttatctatctaaagggctcaaaagacttcctttaaatatcgtaaccccctatgataaattcaactttttgtggtgtggccaaaccattggggaaggggataatggctgtctgtaatagtaatatatagaagagaaatgattctgtccccataaacagtgaatttcaagaaacctgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgacaacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgcccaagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaactggcacagcacggcgtcttgtttgctggagctgggcagtctgaccaaactgaaaatccttgggctacgctggtcaattgtcagtacacacggtaatgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacatactacaacttccccagaattcctccttggatcgcgtcgctggacaagctcacatacctagatatcaatatcgatccagtagaagaggaagcactggagatccttggagaattgccttctttgctgtttctctggctgacgtcgaaatcggctgctccgaaacagcggctcgtcgtaagcagcagcatgtttgtgtgtctgaaggagctccacttcacctgctggagcaatgggcaaggactgatgtttgaggccggggccatgccaaggctcgagaagctgtgggttccgttcgacgcaggcagcggtcttgattctggcatccagcacctctcttccctcacgcatcttgccgtcgagatcatttgcgtcggcgcgaccgctcgggacgtggaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 28 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaatccaggggctcaaggcacgtgttgaagatgcaagcaagcgacgtatgaggtataagcttgatgagcgcaccttcgagcctagcatctcaagggctatcgaccctcgtttgccttcactctatgctgagcctgatgggcttgtcggtattgacaagccaagagacgagctcattaagtgcctaatggaggggatgggtgcatcagtgcagcagcaaaaggtgttatctattgtgggtcctgggggtctcggtaaaactacacttgccaatgaggtgtaccgtaaactggaaggccagttccagtgtcgagcttttgtttccttgtcacaacaaccagatgtgaataagatcttaagaaatatactctctcaagtctgccagcaagagcttcctagcacaagtgtacaggacgagggaaaactcattgacgcaatcagagaagttttaaagaacaagaggtacattcaccaactttgaattagttggctgttggtactgcatcattttttcttcagcattgatgcttatttagtagcctagccattctactatagtctactgttgtttaattaaattaaatatagatgattttgtttcattttaagcatatatatgcaattgtgaatagtgctttgattcacagttcatgaggttatctatctaaagggctcaaaagacttcctttaaatatcgtaaccccctatgataaattcaactttttgtggtgtggccaaaccattggggaaggggataatggctgtctgtaatagtaatatatagaagagaaatgattctgtccccataaacagtgaatttcaagaaacctgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgacaacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgcccaagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttgggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgctggagctgggcagtctgaccaaactgaaaatccttgggctacgctggtcaattgtcagtacacacggtaacgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacatactacaacttccccagaattcctccttggatcgcgtcgctggacaagctcacatacctagatatcaatatcgatccagtagaagaggaagcactggagatccttggagaattgccttctttgctgtttctctggctgacgtcgaaatcggctgctccgaaacagcggctcgtcgtaagcagcagcatgtttgtgcgtctgaaggagctccacttcacctgctggagcaatgggcaaggactgatgtttgaggccggggccatgccaaggctcgagaagctgtgggttccgttcgacgcaggcagcggtcttgattctggcatccagcacctctcctccctcacgcatcttgccgtcgagatcatttgcgtcggcgcgactgctcgggacgtggaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 29 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatggacgactttatgaaaagccttggccaaactgacagtgctcagacagcagggcttgtgcaaagtgtggtccagcagctcaaggcgctgagggcgcgccatcaaatatccagcaaaatccaggggctcaaggcacgtgttgaagatgcaagcaagcgacgtatgaggtataagcttgatgagcgcaccttcgagcctagcatctcaagggctatcaaccctcgtttgccttcactctatgctgagcctgatgggcttgtcggtattgacaagccaagagacgagctcattaagtgcctaatggaggggatgggtgcatcagtgcagcagcaaaaggtgttatctattgtgggtcctgggggtctcggtaaaactacacttgccaatgaggtgtaccgtaaactggaaggccagttccagtgtcgagcttttgtttccttgtcacaacaaccagatgtgaataagatcttaagaaatatactctctcaagtctgccagcaagagcttcctagcacaagtgtacaggacgagggaaaactcattgacgcaatcagagaagttttaaagaacaagaggtacattcaccaactttgaattagttggctgttggtactgcatcattttttcttcagcattgatgcttatttagtagcctagccattctactatagtctactgttgtttaattaaattaaatatagatgattttgtttcattttaagcatatatatgcaattgtgaatagtgctttgattcacagttcatgaggttatctatctaaagggctcaaaagacttcctttaaatatcgtaaccccctatgataaattcaaccttttgtggtgtggccaaaccattggggaaggggataatggctgtctgtaatagtaatatatagaagagaaatgattctgtccccataaacagtgaatttcaagaaacctgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgaccacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgcccaagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgctggagctgggcagtctgaccaaactgaaaatccttgggctacgctggtcaattgtcagtacacacggtaacgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacatactacaacttccccagaattcctccttggatcgcgtcgctggacaagctcacatacctagatatcaatatcgatccagtagaagaggaagcactggagatccttggagaattgccttctttgctgtttctctggctgacgtcgaaatcggctgctccgaaacagcggctcgtcgtaagcagcagcatgtttgtgcgtctgaaggagctccacttcacctgctggagcaatgggcaaggactgatgtttgaggccggggccatgccaaggctcgagaagctgtgggttccgttcgacgcaggcagcggtcttgattctggcatccagcacctctcttccctcacgcatcttgccgtcgagatcatttgcgtcggcgcgactgctcgggacgtggaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 30 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatggacgactttatgaaaagccttggccaaactgacagtgctcagacagcagggcttgtgcaaagtgtggtccagcagctcaaggcgctgagggcgcgccatcaaatatccagcaaaatccaggggctcaaggcacgtgttgaagatgcaagcaagcgacgtatgaggtataagcttgatgagcgcaccttcgagcctagcatctcaagggctatcaaccctcgtttgccttcactctatgctgagcctgatgggcttgtcggtattgacaagccaagagacgagctcattaagtgcctaatggaggggatgggtgcatcagtgcagcagcaaaaggtgttatctattgtgggtcctgggggtctcggtaaaactacacttgccaatgaggtgtaccgtaaactggaaggccagttccagtgtcgagcttttgtttccttgtcacaacaaccagatgtgaataagatcttaagaaatatactctctcaagtctgccagcaagagcttcctagcacaagtgtacaggacgagggaaaactcattgacgcaatcagagaagttttaaagaacaagaggtacattcaccaactttgaattagttggctgttggtactgcatcattttttcttcagcattgatgcttatttagtagcctagccattctactatagtctactgttgtttaattaaattaaatatagatgattttgtttcattttaagcatatatatgcaattgtgaatagtgctttgattcacagttcatgaggttatctatctaaagggctcaaaagacttcctttaaatatcgtaaccccctatgataaattcaaccttttgtggtgtggccaaaccattggggaaggggataatggctgtctgtaatagtaatatatagaagagaaatgattctgtccccataaacagtgaatttcaagaaacctgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgacaacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgcccaagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgctggagctgggcagtctgaccaaactgaaaatccttgggctacgctggtcaattgtcagtacacacggtaacgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacatactacaacttccccagaattcctccttggatcgcgtcgctggacaagctcacatacctagatatcaatatcgatccagtagaagaggaagcactggagatccttggagaattgccttctttgctgtttctctggctgacgtcgaaatcggctgctccgaaacagcggctcgtcgtaagcagcagcatgtttgtgcgtctgaaggagctccacttcacctgctggagcaatgggcaaggactgatgtttgaggccggggccatgccaaggctcgagaagctgtgggttccgttcgacgcaggcagcggtcttgattctggcatccagcacctctcttccctcacgcatcttgccgtcgagatcatttgcgtcggcgcgactgctcgggacgtggaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 31 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaattcaggggctcaaggcacgtgttgaagatgcaagcaagcgacgtataaggtataagcttgatgagcacaccttcgagcctagcatctcaagggctatcgaccctcgtttgccttcactctatgctgagtcagataggcttgtcggtattgacaagccaagagacgagctcattaagtgcctaatggaggggatgggtgcatcagtgcagcagcaaaaggtattatctattgtgggtcctgggggtctcggtaaaactgcacttgccaatgaggtgtaccgtaaactagaaggccagttccagtgtcgagcttttgtttccttgtcacagcaaccagatgtgaataagatcttaagaaatatactctctcaagtctgccagcaagagcttcctagcacaagtgtacaggacgagggaaaactcattgacgcaatcagagaagttttaaagaacaagaggtacgttcaccaactttgaattagttggctgttggtactgcatcattttttttcagcattgatgcttatttagtagcctagccattctactatagtctactgttgtttaattaaattaaattaaatatagatgattttgtttcattttaagcatatatatgcaattgtgaatagtgctttgattcacagttcatgaggttatctatctaaaggactcaaaagacttcctttagatatcgtaacccccaatgataaattcaactttttgtagtgtggccaaaccattggggatggggataatggctgtctgtaatagtaatatatagaagagaaatgattctgtccccataaacagtgaatttcaagaaacttgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgatctgggaagcagaataatgacgacaacacgtagtattgatatagccaagtcatgttgctctcggcgccatgatcgtgtctatgaaataatgcctctcacgacagccaactctaagggtttattttttaaagaatatttggctcagaagatatatgtcctcaattggaagaaatctcctcggaaatattaaaaaaatgtggtggttcaccattagcgattcttacaatagcaagtttattggctaataaagatagcacaaatgaagaatggaagtgggtgtataattcgatcggttcgacactgggaaaggaccccggtgtagaagagatgagaaagatactatctcttagctacgatgatcttcctcaccatttgaagacatgtttattgtatctgagtatatttccggaggactatgagattgagagggatcgattgataaggaggtggatcgctgaaggattcattgatacagatgatggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgcccaagagcacaccatgcttccatcaacagcgatcatttctcattgccgttcgctcagtattgtcgggtatgctgaaaagatgccttctctttcgaagtttcgtgttctgcgagtacttgatattgagaatggcgaggagatggagagcaactgttttgagcatctaaggaagcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgttggagctgggcagtctgaccaaactgaaaatccttgggctacgctggtcaattgtcagtacacacggtaacgaagacacttttgtggttaacttggtatcctcgctgcgcaaactgggcagatccagccttcgatccatatgcattcgtagttatcatggctataccatggagttcttactggactcctggttcccctcccctcatctcatgcaaaagtttcatatgggcgcgtactacaacttccccagagttcctccttggatcgcgtcgctggacaagctcacatacctagatatcaatatcgatccagtagaagaggaagcactggagatccttggagaattgccttctttgctgtttctctggctgacgtcgaaatcggctgctccgaaacagcggctcgtcgtaagcagcagcatgtttgtgcgtctgaaggagctccacttcacctgctggagcaatgggcaaggactgatgtttgaggccggggccatgccgaggctcgagaagctgtgggttccgtttgacgcaggcagcggtcttgattttggcatccagcacctctcttccctcacgcatcttgccgtcgagatcatttgcgtcggcgcgaccgctcgggacgtagaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 32 - DNA, 3306 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaattcaggggctcaaggcacgtgttgaagatgcaagcaagcgacgtataaggtataagcttgatgagcacaccttcgagcctagcatctcaagggctatcgaccctcgtttgccttcactctatgctgagtcagataggcttgtcggtattgacaagccaagagacgagctcattaagtgcctaatggaggggatgggtgcatcagtgcagcagcaaaaggtattatctattgtgggtcctgggggtctcggtaaaactgcacttgccaatgaggtgtaccgtaaactagaaggccagttccagtgtcgagcttttgtttccttgtcacagcaaccagatgtgaataagatcttaagaaatatactctctcaaatctgccagcaagagcttcctagcacaagtgtacaggacgagggaaaactcattgacgcaatcagagaagttttaaagaacaagaggtacgttcaccaactttgaattagttggctgttggtactgcatcattttttttcagcattgatgcttatttagtagcctagccattctactatagtctactgttgtttaattaaattaaattaaatatagatgattttgtttcattttaagcatatatatgcaattgtgaatagtgctttgattcacagttcatgaggttatctatctaaaggactcaaaagacttcctttagatatcgtaacccccaatgataaattcaactttttgtagtgtggccaaaccattggggatggggataatggctgtctgtaatagtaatatatagaagagaaatgattctgtccccataaacagtgaatttcaagaaacttgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgatctgggaagcagaataatgacgacaacacgtagtattgatatagccaagtcatgttgctctcggcgccatgatcgtgtctatgaaataatgcctctcacgacagccaactctaagggtttattttttaaagaatatttggctcagaagatatatgtcctcaattggaagaaatctcctcggaaatattaaaaaaatgtggtggttcaccattagcgattcttacaatagcaagtttattggctaataaagatagcacaaatgaagaatggaagtgggtgtataattcgatcggttcgacactgggaaaggaccccggtgtagaagagatgagaaagatactatctcttagctacgatgatcttcctcaccatttgaagacatgtttattgtatctgagtatatttccggaggactatgagattgagagggatcgattgataaggaggtggatcgctgaaggattcattgatacagatgatggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgcccaagagcacaccatgcttccatcaacagcgatcatttctcattgctgctcgctcagtattgtcgggtatgctgaaaagatgccttctctttcgaagtttcgtgttctgcgagtacttgatattgagaatggcgaggagatggagagcaactgttttgagcatctaaggaagcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgttggagctgggcagtctgaccaaactgaaaatccttgggctacgctggtcaattgtcagtacacacggtaacgaagacacttttgtggttaacttggtatcctcgctgcgcaaactgggcagatccagccttcgatccatatgcattcgtagttatcatggctataccatggagttcttactggactcctggttcccctcccctcatctcatgcaaaagtttcatatgggcgcgtactacaacttccccagagttcctccttggatcgcgtcgctggacaagctcacatacctagatatcaatatcgatccagtagaagaggaagcactggagatccttggagaattgccttctttgctgtttctctggctgacgtcgaaatcggctgctccgaaacagcggctcgtcgtaagcagcagcatgtttgtgcgtctgaaggagctccacttcacctgctggagcaatgggcaaggactgatgtttgaggccggggccatgccgaggctcgagaagctgtgggttccgtttgacgcaggcagcggtcttgattttggcatccagcacctctcttccctcacgcatcttgccgtcgagatcatttgcgtcggcgcgaccgctcgggacgtagaggcgttggaggaggccatcagaggtgcagcccgtctccttccgaaccgccctgcggtggaattccgaacatgggatgatgaaaagatggtggtggaggaggaggagaggcaaggcgtccctgaagaggagatccacgctagcggttga

SEQ ID NO: 33 - DNA, 3310 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaattcaggggctcaaggcacgtgttgaagatgcaagcaagcgacgtataaggtataagcttgatgagcacaccttcgagcctagcatctcaagggctatcgaccctcgtttgccttcactctatgctgagtcagataggcttgtcggtattgacaagccaagagacgagctcattaagtgcctaatggaggggatgggtgcatcagtgcagcagcaaaaggtattatctattgtgggtcctgggggtctcggtaaaactgcacttgccaatgaggtgtaccgtaaactagaaggccagttccagtgtcgagcttttgtttccttgtcacagcaaccagatgtgaataagatcttaagaaatatactctctcaaatctgccagcaagagcttcctagcacaagtgtacaggacgagggaaaactcattgacgcaatcagagaagttttaaagaacaagaggtacgttcaccaactttgaattagttggctgttggtactgcatcattttttttcagcattgatgcttatttagtagcctagccattctactatagtctactgttgtttaattaaattaaattaaatatagatgattttgtttcattttaagcatatatatgcaattgtgaatagtgctttgattcacagttcatgaggttatctatctaaaggactcaaaagacttcctttagatatcgtaacccccaatgataaattcaactttttgtagtgtggccaaaccattggggatggggataatggctgtctgtaatagtaatatatagaagagaaatgattctgtccccataaacagtgaatttcaagaaacttgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgacaacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgccctagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgctggagctgggcagtctgaccaaactgaaaatccttgggctacgctggtcaattgtcagtacacacggtaacgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacgtactacaacttccccagagttcctccttggatcgcgtcgctggacaagctcacatacctagatatcaatatcgatccagtagaagaggaagcactggagatccttggagaattgccttctttgctgtttctctggctgacatcgaaatcggctgctcccaaacagcggctcgtcgtaagcagcagcatgtttgtgcgtctgaaggagctccacttcacctgctggagcaatgggcaaggactgatgtttgaggccggggccatgccgaggctcgagaagctgtgggttccgtttgacgcaggcagcggtcttgattttggcatccagcacctctcttccctcacgcatcttgccgtcgagatcatttgcgtcggcgcgaccgctcgggacgtagaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 34 - DNA, 3311 bp, Hordeum vulgare
atggtgagcgccttggcaggggtgatgacctctgtcatcggcaagctcaccgccctgctcggggaggagtacgcaaagctgaaaggtgtgcacagggaggtggagttcatgaaagatgagctgagcagcatgaacgcgctccttcagaggctggcagaggcggaccgtgatcttgatgtgcagacgaaggaatggagggaccaagttcgggagatgtcttacgacattgaggattgcatagacgactttatgaaaagccttggccaaactgacagtgctcagacagcagggcttgtgcaaagtgtggtccagcagctcaaggcgctgagggcgcgccatcaaatatccagcaaaattcaggggctcaaggcacgtgttgaagatgcaagcaagcgacgtataaggtataagcttgatgagcacaccttcgagcctagcatctcaagggctatcgaccctcgtttgccttcactctatgctgagtcagataggcttgtcggtattgacaagccaagagacgagctcattaagtgcctaatggaggggatgggtgcatcagtgcagcagcaaaaggtattatctattgtgggtcctgggggtctcggtaaaactgcacttgccaatgaggtgtaccgtaaactagaaggccagttccagggtcgagcttttgtttccttggtcacagcaaccagatgtgaataagatcttaagaaatatactctctcaaatctgccagcaagagcttcctagcacaagtgtacaggacgagggaaaactcattgacgcaatcagagaagttttaaagaacaagaggtacgttcaccaactttgaattagttggctgttggtactgcatcattttttttcagcattgatgcttatttagtagcctagccattctactatagtctactgttgtttaattaaattaaattaaatatagatgattttgtttcattttaagcatatatatgcaattgtgaatagtgctttgattcacagttcatgaggttatctatctaaaggactcaaaagacttcctttagatatcgtaacccccaatgataaattcaactttttgtagtgtggccaaaccattggggatggggataatggctgtctgtaatagtaatatatagaagagaaatgattctgtccccataaacagtgaatttcaagaaacttgaagccctatatcattctcatgatttactaacatttgacgacaaggaaacatgccattttatttatttgttcacatattttccttattcatgagatttgcaaaacatgcactttgcaggtacttagttgttattgatgatatatggagtactcaagcatggaagattatcaaatgttctttgtttctgaatgacctgggaagcagaataatgacgacaacacgtagtattgatatagccaagtcatgttgctctcggcgccatgatcgtgtctatgaaataatgcctctgacgacagccaactctaagggtttattttttaaacgaatatttggctcagaagatatatgtcctcctcaattggaagaaatctcctcggaaatattaaaaaaatgtggtggttcaccattagcgattcttacaatagcaagtttattggccaataaagatagcacaaatgaagaatggaagtgggtgtataattcgatcggttcgacactgggaaaggaccccggtgtagaagagatgagaaggatactatctcttagctacgatgatcttcctcaccatttgaagacatgtttattgtatctgagtatatttccggaggactatgagattgagagggatcgattgataaggaggtggatcgctgaaggattcattgatacagatggtggacgagatttggaggaaataggagagtgttattttaatgatcttatcaatagaagtatgcttgagccagtgaaaatccaatatgacggtcaagtcgtttcatgccgagtgcatgatatgattctggatctccttgcatctaagtcaattgaagaaaactttgccaccttctctggtaaccaaaatgagatattagtccttcggcataagatccgtaggctatctctcaattattatgccctagagcacaccatgcttccatcaacagcgatcatttctcattgccgttcgctcagtattgtcgggtatgctgaaaagatgccttctctttcgaagtttcgtgttctgcgagtacttgatattgagaatggtgaggagatggagagcaactgttttgagcatctaaggacgcttttccagttgaggtatttgcgactccacgttagaagtatttctgcactccctgagcagttaggagaactacagcatttgaggactctggatatgggctggacaaagatcacaaaaatgcccaaaagcattgttcagctgcaacatttgacatgtttgcgcgtcagtaatttggaattacctgaagggattgggaatctgcaagctctgcaggagctatcagatatcaaagtcaaccggcacagcacggcgtcttgtttgctggagctgggcagtctgaccaaactgaaaatccttgggctacgctggtcaattgtcagtacacacggtaacgaagacacttttgtggataacttggtatcctcgctgcgcaaactgggcagatccagccttcgatccatatgcattcgtagttatcatggctataccatggagttcttactggactcctggttcccctcccctcatctcatgcaaaagtttcagatgggcacgtactacaacttccccagagttcctccttggatcgcgtcgctggacaagctcacatacctagatatcaatatcgatccagtagaagaggaagcactggagatccttggagaattgccttctttgctgtttctctggctgacatcgaaatcggctgctcccaaacagcggctcgtcgtaagcagcagcatgtttgtgcgtctgaaggagctccacttcacctgctggagcaatgggcaaggactgatgtttgaggccggggccatgccgaggctcgagaagctgtgggttccgtttgacgcaggcagcggtcttgattttggcatccagcacctctcttccctcacgcatcttgccgtcgagatcatttgcgtcggcgcgaccgctcgggacgtagaggcgttggaggaggccatcagaggtgcagcccgtctccttccgaaccgccctgcggtggaattccgaacatgggatgatgaaaagatggtggtggaggaggaggaggggcaaggcgtccctgaagaggagatccacgctagcggttga

SEQ ID NO: 35 - DNA, 3306 bp, Hordeum vulgare; identical to SEQ ID NO: 26

SEQ ID NO: 36 - DNA, 3306 bp, Hordeum vulgare; identical to SEQ ID NO: 27

SEQ ID NO: 37 - DNA, 3306 bp, Hordeum vulgare; identical to SEQ ID NO: 28

SEQ ID NO: 38 - protein, 5 aa, Unknown
  Description of Unknown “EDVID” motif sequence
EDVID

SEQ ID NO: 39 - DNA, 1461 bp, Zea mays
atgggctgcttcccgtgcttcggctccacgcgcgaggaggagctcaagtactacggatccaagggccccggcggaggcaatggcggtggtgggcgagcgaccgcgtcctcgtcctcgtccgctgcagctggtggtggaggcggccgggtagaagaggccgtggtggcgccgccgcgtgctcagaggggccccgcaggggccgacaagacacgagctaaaggcaatgctggctcgaagaaggagctttcagtactcagggacgccagtggcaatgtcatctctgctcagaccttcaccttccgccagcttgcagccgcaacgaagaactttagagatgaatgcttcattggggagggagggtttgggcgcgtttacaagggccgccttgacatgggccaggttgttgctatcaaacagctgaatagggatggtaatcaaggaaacaaagaatttctagtggaagttctcatgcttagtttgctgcatcatcaaaaccttgtcaatttggttggttattgtgctgatggagatcaacgccttctcgtgtatgagtacatgccacttggatcattggaggaccatttgcatgatctccctcctgacaaggagcctttggattggaacactaggatgaaaattgctgcgggtgctgctaaagggctggagtacctgcatgacaaggcacagccaccagttatttacagggatttcaagtcatcaaatattctattgggcgagggcttccatccaaagctatcagacttcggtcttgctaagttgggtcctgttggtgacaagtctcatgtctcaacgcgtgttatgggaacatatggttattgtgctccagaatatgctatgacaggacaacttacagttaagtcagatgtttacagctttggagttgtcttgctcgagctgattactggccgaaaggccattgacagcaccagaccagcgtcagagcaaaaccttgtgtcatgggcacggccccttttcaatgacaggcgcaagctcccaaagatggctgacccgggcctggaggggcagtttcctacacggggactttaccaggcgcttgcggtggcatcaatgtgcatccagtcagaggctgcatcgcgcccactcatcgccgacgttgtgactgctctgtcataccttgcaaaccagatttatgatcctagcttagcgcacgcatccaagaaagcaggcggcagcgaccagcggaaccgggtcggtgacagtggaagggcgctttccaagaatgacgatgcaggcagctctggccacaggtcgccgagcaaggaccgggccgactcccccagagagcagttcccgggggctgcgaacaggggccaggacagggagcgaatggtggcagaggcaaagatgtggggcgagaactggcgggagaagcggcgagctgctcaggggagcctggattctccgaccggaggcgggtag

SEQ ID NO: 40 - protein, 486 aa, Zea mays
MGCFPCFGSTREEELKYYGSKGPGGGNGGGGRATASSSSSAAAGGGGGRVEEAVVAPPRAQRGPAGADKTRAKGNAGSKKELSVLRDASGNVISAQTFTFRQLAAATKNFRDECFIGEGGFGRVYKGRLDMGQVVAIKQLNRDGNQGNKEFLVEVLMLSLLHHQNLVNLVGYCADGDQRLLVYEYMPLGSLEDHLHDLPPDKEPLDWNTRMKIAAGAAKGLEYLHDKAQPPVIYRDFKSSNILLGEGFHPKLSDFGLAKLGPVGDKSHVSTRVMGTYGYCAPEYAMTGQLTVKSDVYSFGVVLLELITGRKAIDSTRPASEQNLVSWARPLFNDRRKLPKMADPGLEGQFPTRGLYQALAVASMCIQSEAASRPLIADVVTALSYLANQIYDPSLAHASKKAGGSDQRNRVGDSGRALSKNDDAGSSGHRSPSKDRADSPREQFPGAANRGQDRERMVAEAKMWGENWREKRRAAQGSLDSPTGGG

SEQ ID NO: 41 - protein, 486 aa, Artificial Sequence
  Description of Artificial Sequence Synthetic polypeptide
MGCFPCFGSTREEELKYYGSKGPGGGNGGGGRATASSSSSAAAGGGGGRVEEAVVAPPRAQRGPAGADKTRAKGNAGSKKELSVLRDASGNVISAQTFTFRQLAAATKNFRDECFIGEGGFGRVYKGRLDMGQVVAIKQLNRDGNQGNKEFLVEVLMLSLLHHQNLVNLVGYCADGDQRLLVYEYMPLGSLEDHLHDLPPDKEPLDWNTRMKIAAGAAKGLEYLHDKAQPPVIYRDFKSSNILLGEGFHPKLSDFGLAKLGPVQYCVYESTRVMGTYGYCAPEYAMTGQLTVKSDVYSFGVVLLELITGRKAIDSTRPASEQNLVSWARPLFNDRRKLPKMADPGLEGQFPTRGLYQALAVASMCIQSEAASRPLIADVVTALSYLANQIYDPSLAHASKKAGGSDQRNRVGDSGRALSKNDDAGSSGHRSPSKDRADSPREQFPGAANRGQDRERMVAEAKMWGENWREKRRAAQGSLDSPTGGG

SEQ ID NO: 42 - DNA, 1431 bp, Triticum aestivum
atgggttgcttcccgtgcttcgattcgggctccgacggggagctgctctaccccaagcagggcggcggaggcggcggaaatggcacgggcggacggactgcggccgcggcatcgtcctccggcgtcggcgcccgcgaggagagacccatggtcccgccgcgcgtcgagaagctccccgcaggggctgagaaggcaagggcaaaaggcaatgccggaatgaaggagctttcagatctcagggatgccaatggcaatgtcctctctgcgcagacgttcaccttccgccagcttacagctgccacgaggaacttcagggaggaatgcttcattggggagggagggttcggacgtgtttacaagggccgtcttgatggaggccaggttgttgctataaagcagctcaatagggatggaaaccaaggaaacaaagaatttctggtggaggtccttatgctcagtttgctgcatcatcaaaaccttgttaatttggttggttattgcgctgatggagagcaacgccttctggtgtatgagtatatgccccttggatcattggaagaccatctccatgatctccctcctgataaggaaccgttggactggaacactaggatgaaaattgcagctggtgctgctaaagggttggaatacctccatgacaaggcacaaccaccagttatatatagagacttcaagtcatcaaatattctattgggtgatgatttccatccaaagctgtcagactttggtctcgctaaattgggtcctgttggtgacaagtctcatgtctctacacgtgtgatgggaacatacggctattgtgctccagaatatgctatgacagggcaacttacagtcaagtcagatgtctatagctttggagtggtgttgcttgagttgattactggccggaaggccattgacagcaccagacctcatggggaacaaaacctcgtgtcatgggcacgccctcttttcaatgacaggcggaagctcccaaagatggctgatccagggctgcagggacgatatcccatgcgtgggctctaccaagccctcgctgtggcgtcaatgtgtattcagtcagaggctgcttcgcgaccacttatcgctgatgttgtgactgctctttcctacttggcgtcccaaatttatgatcctaatgcgatccatgcctcgaaaaaggcaggtggcgaccagcgaagtagggtttctgatagtggaaggacgctcctgaagaatgatgaggcaggcagctcaggacacaagtcggatcgggatgattcccccagggagcctcctccggggatccttaatgacagggagaggatggtggctgaggcgaagatgtggggtgccaacctgcgggagaagacgcgtgctgctgccaatgcacaggggagcctcgattctccaactgaaaccggatag

SEQ ID NO: 43 - protein, 476 aa, Triticum aestivum
MGCFPCFDSGSDGELLYPKQGGGGGGNGTGGRTAAAASSSGVGAREERPMVPPRVEKLPAGAEKARAKGNAGMKELSDLRDANGNVLSAQTFTFRQLTAATRNFREECFIGEGGFGRVYKGRLDGGQVVAIKQLNRDGNQGNKEFLVEVLMLSLLHHQNLVNLVGYCADGEQRLLVYEYMPLGSLEDHLHDLPPDKEPLDWNTRMKIAAGAAKGLEYLHDKAQPPVIYRDFKSSNILLGDDFHPKLSDFGLAKLGPVGDKSHVSTRVMGTYGYCAPEYAMTGQLTVKSDVYSFGVVLLELITGRKAIDSTRPHGEQNLVSWARPLFNDRRKLPKMADPGLQGRYPMRGLYQALAVASMCIQSEAASRPLIADVVTALSYLASQIYDPNAIHASKKAGGDQRSRVSDSGRTLLKNDEAGSSGHKSDRDDSPREPPPGILNDRERMVAEAKMWGANLREKTRAAANAQGSLDSPTETG

SEQ ID NO: 44 - protein, 476 aa, Artificial Sequence
  Description of Artificial Sequence Synthetic polypeptide
MGCFPCFDSGSDGELLYPKQGGGGGGNGTGGRTAAAASSSGVGAREERPMVPPRVEKLPAGAEKARAKGNAGMKELSDLRDANGNVLSAQTFTFRQLTAATRNFREECFIGEGGFGRVYKGRLDGGQVVAIKQLNRDGNQGNKEFLVEVLMLSLLHHQNLVNLVGYCADGEQRLLVYEYMPLGSLEDHLHDLPPDKEPLDWNTRMKIAAGAAKGLEYLHDKAQPPVIYRDFKSSNILLGDDFHPKLSDFGLAKLGPVQYCVYESTRVMGTYGYCAPEYAMTGQLTVKSDVYSFGVVLLELITGRKAIDSTRPHGEQNLVSWARPLFNDRRKLPKMADPGLQGRYPMRGLYQALAVASMCIQSEAASRPLIADVVTALSYLASQIYDPNAIHASKKAGGDQRSRVSDSGRTLLKNDEAGSSGHKSDRDDSPREPPPGILNDRERMVAEAKMWGANLREKTRAAANAQGSLDSPTETG

SEQ ID NO: 45 - DNA, 1440 bp, Hordeum vulgare
atgggttgcttcccgtgcttcgattcgagctccgacggggagctgctctaccccaagcagggcggcggcggcggcggcggcggcggaaatggcacaggcggacggactgcggctgcggcatcgtcctccggcgtcggcgcccgcgaggagagacccatggtcccaccgcgcgtcgagaagctccccgcaggggctgagaaggcaagggcaaaaggcaatgccggaatgaaggagctttcagatctcagggatgccaatggtaatgtcctctctgcgcagacgttcaccttccgtcagcttaccgctgccacgaggaacttcagggaggaatgcttcattggggagggagggtttggacgtgtttacaagggccgtcttgatggaggccaggttgttgctataaagcagctcaatagggatggaaatcaaggaaacaaagaatttctggtggaggtcctcatgctcagtttgctgcatcatcaaaaccttgttaatttggttggttattgcgctgatggagaacaacgccttctggtgtatgagtatatgccccttggatcattggaagaccatctccatgatctccctcctgataaggaaccgttggactggaacactaggatgaaaatcgcagctggtgctgctaaagggctggagtacctccatgacaaggcacaaccaccagttatatatagagatttcaagtcatcaaatattctattgggtgacgatttccatccaaagctgtcagactttggtctcgctaaattgggtcctgttggtgacaagtctcatgtctctacacgtgtgatgggaacatatggctactgtgctccagaatatgctatgacagggcaacttacagtcaagtcagatgtctatagctttggagtggtgttgcttgagttgattactggccggaaggctatcgacagcaccagacctcatggggagcaaaacctcgtgtcatgggcgcgccctcttttcaatgacaggcggaagctcccaaagatggctgatccagggcttcagggacgatatcccatgcgtgggctataccaagcacttgctgtggcgtcaatgtgtattcagtcagaggctgcttcgcgaccacttatcgctgatgttgtgactgctctttcgtacctggcgtcccaaatttatgatcctaatgcgatccatgcctcaaaaaaggcaggtggcgatcagcgaagtagggtttctgacagtggaagggcgctcctgaagaatgatgaggcaggcagctctggacacaagtcggatcgggatgattcccccagggagcctcctccagggatccttaatgacagggagcggatggtggctgaggcgaagatgtggggtgcgaacctgcgggagaagacgcgtgctgctgccaatgcacaggggagcctcgattctccaactgaaaccggataa

SEQ ID NO: 46 - protein, 479 aa, Hordeum vulgare; identical to SEQ ID NO: 23

SEQ ID NO: 47 - protein, 479 aa, Artificial Sequence
  Description of Artificial Sequence Synthetic polypeptide
MGCFPCFDSSSDGELLYPKQGGGGGGGGGNGTGGRTAAAASSSGVGAREERPMVPPRVEKLPAGAEKARAKGNAGMKELSDLRDANGNVLSAQTFTFRQLTAATRNFREECFIGEGGFGRVYKGRLDGGQVVAIKQLNRDGNQGNKEFLVEVLMLSLLHHQNLVNLVGYCADGEQRLLVYEYMPLGSLEDHLHDLPPDKEPLDWNTRMKIAAGAAKGLEYLHDKAQPPVIYRDFKSSNILLGDDFHPKLSDFGLAKLGPVQYCVYESTRVMGTYGYCAPEYAMTGQLTVKSDVYSFGVVLLELITGRKAIDSTRPHGEQNLVSWARPLFNDRRKLPKMADPGLQGRYPMRGLYQALAVASMCIQSEAASRPLIADVVTALSYLASQIYDPNAIHASKKAGGDQRSRVSDSGRALLKNDEAGSSGHKSDRDDSPREPPPGILNDRERMVAEAKMWGANLREKTRAAANAQGSLDSPTETG

SEQ ID NO: 48 - DNA, 1581 bp, Oryza sativa
atgggctgcttctcgtgcttcgactcgcccgcggaggagcagctgaaccccaaggtgggagggccgtacggcggcggctcctcctcctccgccgccgcggccgcgtacggcggcggcggcggttccagcgctggtcggcatggggagaggggcggtggttacccggacctgcaccaccaccaccagcagcagcagctgcccatggcagcgccgcgcgtcgagaagctctccgcaggggctgagaagacgagggtgaagagcaatgcaatactaagggaaccttctgcgcccaaggatgccaatgggaatgtaatatcagcacagactttcaccttccgagaactcgcgaccgccaccaggaatttcaggccagagtgcttcctgggcgaaggaggctttgggcgcgtttataagggccgcctcgagagcactggccaggttgtggctataaagcagcttaatagagatgggcttcaaggaaacagagaatttctagtggaagttctaatgctcagtttgttacatcatcaaaatcttgttaatctgattggctattgtgctgacggagatcaacgtcttcttgtttatgaatacatgcactttggatcattggaagatcatttgcatgatctacctcctgataaggaagcattggattggaacacaaggatgaaaattgcggctggtgctgctaagggactagagtaccttcatgacaaagcaaatccaccagttatttatagagacttcaagtcatcaaacattctcttagatgagagtttccatccaaagctgtctgactttggtcttgcaaagttgggtcctgttggtgacaaatctcatgtctctactcgtgtgatgggaacatatggatattgtgctcctgagtatgctatgacaggacagttgacagttaagtctgatgtatatagctttggagttgtgttgcttgagttgattactggtcgtcgggcgatcgacagcactaggccacatggggagcaaaatcttgtctcatgggcacgacctcttttcaacgacagaagaaagctcccaaaaatggctgacccaagactggagggacgataccccatgcgtggactttaccaagcacttgcagtggcatctatgtgcattcagtcggaggctgcgtcgcgtccacttattgcagatgttgtgactgctctttcctacctggcatctcaatcatatgaccctaatgcggctcatgcttcaagaaagccaggtggcgatcagcgaagcaaggttggcgagaacggcagggtagtctccaggaacgacgaagccagcagctctggccacaagtcaccaaacaaggacagggaggattccccgaaagagcccccaggtatcctgaacaaggatttcgacagggagcggatggtggcggaggcaaagatgtggggcgacagggaacggatggtggctgaggcaaagatgtggggtgatagggagcggatggtagccgaggcaaagatgtggggcgagaattggcgggataagagacgtgcgatcgagaacgggcaggggagtctggattccccaactgaaaatggctag

SEQ ID NO: 49 - protein, 526 aa, Oryza sativa
MGCFSCFDSPAEEQLNPKVGGPYGGGSSSSAAAAAYGGGGGSSAGRHGERGGGYPDLHHHHQQQQLPMAAPRVEKLSAGAEKTRVKSNAILREPSAPKDANGNVISAQTFTFRELATATRNFRPECFLGEGGFGRVYKGRLESTGQVVAIKQLNRDGLQGNREFLVEVLMLSLLHHQNLVNLIGYCADGDQRLLVYEYMHFGSLEDHLHDLPPDKEALDWNTRMKIAAGAAKGLEYLHDKANPPVIYRDFKSSNILLDESFHPKLSDFGLAKLGPVGDKSHVSTRVMGTYGYCAPEYAMTGQLTVKSDVYSFGVVLLELITGRRAIDSTRPHGEQNLVSWARPLFNDRRKLPKMADPRLEGRYPMRGLYQALAVASMCIQSEAASRPLIADVVTALSYLASQSYDPNAAHASRKPGGDQRSKVGENGRVVSRNDEASSSGHKSPNKDREDSPKEPPGILNKDFDRERMVAEAKMWGDRERMVAEAKMWGDRERMVAEAKMWGENWRDKRRAIENGQGSLDSPTENG

SEQ ID NO: 50 - protein, 526 aa, Artificial Sequence
  Description of Artificial Sequence Synthetic polypeptide
MGCFSCFDSPAEEQLNPKVGGPYGGGSSSSAAAAAYGGGGGSSAGRHGERGGGYPDLHHHHQQQQLPMAAPRVEKLSAGAEKTRVKSNAILREPSAPKDANGNVISAQTFTFRELATATRNFRPECFLGEGGFGRVYKGRLESTGQVVAIKQLNRDGLQGNREFLVEVLMLSLLHHQNLVNLIGYCADGDQRLLVYEYMHFGSLEDHLHDLPPDKEALDWNTRMKIAAGAAKGLEYLHDKANPPVIYRDFKSSNILLDESFHPKLSDFGLAKLGPVQYCVYESTRVMGTYGYCAPEYAMTGQLTVKSDVYSFGVVLLELITGRRAIDSTRPHGEQNLVSWARPLFNDRRKLPKMADPRLEGRYPMRGLYQALAVASMCIQSEAASRPLIADVVTALSYLASQSYDPNAAHASRKPGGDQRSKVGENGRVVSRNDEASSSGHKSPNKDREDSPKEPPGILNKDFDRERMVAEAKMWGDRERMVAEAKMWGDRERMVAEAKMWGENWRDKRRAIENGQGSLDSPTENG

SEQ ID NO: 51 - DNA, 1539 bp, Sorghum bicolor
atgggctgcttctcgtgcttcgactccccggcggatgagcagctcaacccaaaattcggtggcgccggtgggtacggcggcggcacgtccgctgccgccgcggcgtacggagccggcgccggcgccggcgtcggccggcacgggggcaggggcgggtacccggacctgcagcaggcgcccatggcggcgccgcgcgtcgagaagttctccgcagcggctgagaaagcaagagttaagagcaatgtgctcaccaaggaggcttcggtgccaaaggatgccaacggcaatgccatctcggcgcagactttcaccttccgtgagctcgccaccgctaccaggaacttcaggcctgagtgcttcctgggagagggaggttttggacgtgtttacaagggacgccttgagagcacaggccaggttgttgctataaagcagcttaacagggatgggcttcaaggaaacagagaatttctagtagaagttctcatgctcagcttactacatcatcaaaacctggttaatttgattggttattgtgctgatggagaccagcgacttcttgtttatgaatatatgccctccggatcactggaagatcatttgcatgatctacctcttgataaggaggccttggactggaacaccaggatgaaaattgcagcaggtgctgccaaaggacttgagtaccttcatgacaaagctaatccgccagttatttatagggatttcaagtcatcaaacattctgttggatgaaagtttccacccgaagctgtctgactttggacttgctaagttgggtccagttggcgacaaatcacatgtctcaacacgtgtaatgggtacatatggttattgtgcaccagaatatgctatgacagggcagctaacagtgaagtctgatgtgtatagctttggggttgtcttgctagagttgattactggtcgtagggctatcgacagcaccagaccacatggagaacaaaatcttgtctcatgggcacgtcctcttttcaatgacagaagaaagctccctaagatggctgacccaaggctggaagggcgataccccatgcgtgggctttaccaagccctcgcggtggcttccatgtgcattcagtcggaggctgcttcacgcccactgattgcagatgtggtgactgctctgtcctacttggcatcccagcaatatgatcccaacacagctcttgcttcaaggaagccaggtggcgatcaacgaagcaggcctggtgagaacggcagggtggtttccaggaatgacgagactggaagctcgggccacaaatcacctggcaaggaccgggaggactcacccagggacctcccagcgatcctgaacaaggacctggaacgtgagcgcatggtggcggaggcgaagatgtggggcgaccgggagcgcatggttgccgaggcgaagatgtggggcgaccgggagcgcatggtggccgaggcgaagatgtggggcgagaattggcgtgataagcggcgtgcagagaatgggcaggggagcctggactag

SEQ ID NO: 52 - protein, 512 aa, Sorghum bicolor
MGCFSCFDSPADEQLNPKFGGAGGYGGGTSAAAAAYGAGAGAGVGRHGGRGGYPDLQQAPMAAPRVEKFSAAAEKARVKSNVLTKEASVPKDANGNAISAQTFTFRELATATRNFRPECFLGEGGFGRVYKGRLESTGQVVAIKQLNRDGLQGNREFLVEVLMLSLLHHQNLVNLIGYCADGDQRLLVYEYMPSGSLEDHLHDLPLDKEALDWNTRMKIAAGAAKGLEYLHDKANPPVIYRDFKSSNILLDESFHPKLSDFGLAKLGPVGDKSHVSTRVMGTYGYCAPEYAMTGQLTVKSDVYSFGVVLLELITGRRAIDSTRPHGEQNLVSWARPLFNDRRKLPKMADPRLEGRYPMRGLYQALAVASMCIQSEAASRPLIADVVTALSYLASQQYDPNTALASRKPGGDQRSRPGENGRVVSRNDETGSSGHKSPGKDREDSPRDLPAILNKDLERERMVAEAKMWGDRERMVAEAKMWGDRERMVAEAKMWGENWRDKRRAENGQGSLD

SEQ ID NO: 53 - protein, 512 aa, Artificial Sequence
  Description of Artificial Sequence Synthetic polypeptide
MGCFSCFDSPADEQLNPKFGGAGGYGGGTSAAAAAYGAGAGAGVGRHGGRGGYPDLQQAPMAAPRVEKFSAAAEKARVKSNVLTKEASVPKDANGNAISAQTFTFRELATATRNFRPECFLGEGGFGRVYKGRLESTGQVVAIKQLNRDGLQGNREFLVEVLMLSLLHHQNLVNLIGYCADGDQRLLVYEYMPSGSLEDHLHDLPLDKEALDWNTRMKIAAGAAKGLEYLHDKANPPVIYRDFKSSNILLDESFHPKLSDFGLAKLGPVQYCVYESTRVMGTYGYCAPEYAMTGQLTVKSDVYSFGVVLLELITGRRAIDSTRPHGEQNLVSWARPLFNDRRKLPKMADPRLEGRYPMRGLYQALAVASMCIQSEAASRPLIADVVTALSYLASQQYDPNTALASRKPGGDQRSRPGENGRVVSRNDETGSSGHKSPGKDREDSPRDLPAILNKDLERERMVAEAKMWGDRERMVAEAKMWGDRERMVAEAKMWGENWRDKRRAENGQGSLD

SEQ ID NO: 54 - protein, 7 aa, Artificial Sequence
  Description of Artificial Sequence Synthetic peptide
QYCVYES

SEQ ID NO: 55 - protein, 7 aa, Artificial Sequence
  Description of Artificial Sequence Synthetic peptide
ESVLSQS